(19) United States
(12) Patent Application Publication
(10) Pub. No.: US 2025/0145990 A1
(43) Pub. Date: May 8, 2025

(54) TRANS-SPLICING RNA (TSRNA)

(71) Applicant: National University of Singapore, Singapore (SG)

(72) Inventors: Pei She Loh, Singapore (SG); Volker Patzel, Singapore (SG)

(73) Assignee: National University of Singapore, Singapore (SG)

(21) Appl. No.: 18/730,291

(22) PCT Filed: Jan. 18, 2023
(86) PCT No.: PCT/SG2023/050034

§ 371 Date: Jul. 18, 2024

(30) Foreign Application Priority Data
Jan. 18, 2022 (GB) 2200546.6

(51) Int. Cl. C12N 15/113; A61K 45/06; A61P 35/00; C12N 15/115; C12N 15/85

(52) U.S. Cl. CPC C12N 15/113; A61K 45/06; A61P 35/00; C12N 15/115; C12N 15/85; C12N 2310/11; C12N 2310/12; C12N 2310/16; C12N 2830/42

(57) ABSTRACT

Trans-splicing RNA (tsRNA) The invention concerns a trans-splicing RNA (tsRNA) molecule comprising at least one or multiple unstructured target binding domains complementary to at least one or multiple precursor messenger RNA (pre-mRNA) targets and adapted to prevent off-target trans-splicing by the inclusion of a safety domain; a cell or vector or therapeutic or composition or pharmaceutical composition comprising said tsRNA; and a method for killing cells or treating a disease or for imaging or for a cosmetic application using said tsRNA. The invention has use in the medical, cosmetic, and veterinary field.

### FIELD OF THE INVENTION

The invention concerns a trans-splicing RNA (tsRNA) molecule comprising at least one or multiple unstructured target binding domains complementary to at least one or multiple precursor messenger RNA (pre-mRNA) targets and adapted to prevent off-target trans-splicing by the inclusion of a safety domain; a cell or vector or therapeutic or composition or pharmaceutical composition comprising said tsRNA; and a method for killing cells or treating a disease or for imaging or for a cosmetic application using said tsRNA. The invention has use in the medical, cosmetic, and veterinary field.

### BACKGROUND OF THE INVENTION

Spliceosome-meditated RNA trans-splicing (SMART) is the process by which two distinct precursor messenger RNAs (pre-mRNAs), or other spliceable RNAs, are joint in trans to generate a chimeric RNA molecule in the nucleus that, after nuclear export, triggers the formation of a chimeric protein in the cytoplasm. This technology can be used to repair defective RNA, e.g., by replacing a mutated with an intact exon, or to label an endogenous message with a functional sequence. This functional sequence can trigger the expression of a death signal to kill the targeted cell, of a fluorescent protein to image the targeted cells, of a therapeutic or cosmetic protein to treat or cure the targeted cells, or of any gene of interest to program/re-program the targeted cell.

Trans-splicing-based repair is difficult to achieve because durable repair requires the trans-splicing RNA to be delivered continuously or to be expressed endogenously after genomic integration, it also requires precise splicing towards the intended splice sites within the target, and it must be efficient enough to trigger the therapeutic phenotype despite strong competition with regular cis-splicing.

Trans-splicing-based labelling with a functional sequence concerns, for example, an RNA coding for a fluorescent protein to monitor the expression of genes in living cells or a death signal to selectively trigger death of cells expressing aberrant transcripts in a suicide gene therapy approach. An aberrant transcript can be a biomarker for diseased cells such as transcripts of oncogenes specific for cancer or viral transcripts. The death signal can be triggered by a) a direct signal such as a toxin e.g., diphtheria or the cholera toxin, b) an apoptotic or necrotic gene such as a caspase, or c) an enzyme such as the herpes simplex virus thymidine kinase (HSVtk) that triggers a death signal upon co-delivery of a drug like ganciclovir (GCV). Direct toxins a) or apoptotic signals b) can immediately trigger cell death which, unfortunately, increases the risks involved with unspecific targeting or off-targeting. This makes the regulatory approval of such technologies problematical. In contrast, the use of a combination of two components c) which, by themselves, are not toxic to the cells represents a much safer approach.

As a therapy, trans-splicing-triggered cell death is easier to achieve than trans-splicing-based repair because the trans-splicing construct needs to be delivered only once into the target cells and so long-term expression is not necessary. Moreover, alternative on-target trans-splicing, i.e., trans-splicing towards the right target but involving any splice sites of that target, is not disadvantageous but instead contributes to a target-specific death signal and trans-splicing doesn't need to be highly efficient to trigger a signal that is strong enough to kill the targeted cells.

Based on the HSVtk/GCV-system we have developed an efficient trans-splicing-based suicide gene therapy approach. We have designed new trans-splicing RNAs both for 5′ and 3′ exon replacement (ER), i.e., for attaching a suicide gene or a component of a suicide gene system such, as the HSVtk, either to the 5′ or 3′ end of the target message. We have investigated RNA structure design to improve both on-target activity and specificity of trans-splicing RNA (tsRNA).

The efficiency of methods such as gene therapy of inherited and acquired genetic diseases, genetic vaccination, stem cell programming, somatic cell reprogramming, immunotherapy and manipulation of protein expression and trans-splicing-based suicide gene therapy in vivo is dependent on the delivery of recombinant DNA into primary cells ex vivo or in vivo in order to trigger the expression of non-coding RNAs or proteins. In primary cells, the expression of recombinant foreign episomal DNA (such as plasmids) is silenced within 24 hours post-delivery, independent of the route of delivery. The mechanisms underlying this effect are poorly understood. Only integrating viral delivery vectors, such as retroviral, lentiviral, and AAV vectors have been successfully used to trigger medium and long-term expression in primary cells. These vectors, however, are costly considering current good Manufacturing Practise (CGMP) production standards. It is considered to be several orders of magnitude more expensive to produce viral vectors under cGMP standards than generating an equivalent quantity of ‘naked’ genetic material. In addition, viral vectors harbour safety risks and concerns which are associated (i) with negative interference of the integrated foreign DNA at the loci of integration (e.g., disruption of gene function and regulation), and (ii) with the involvement of components originating from pathogenic viruses. Alternatively, the direct delivery of functional RNA into primary cells results in rapid degradation and providing only short-term effects. Hence, there is a strong desire for the development of novel genetic vectors that escape transgene silencing.

The present disclosure allows for sustained and safe transgene expression in primary cells solving the problem of transgene silencing.

Novel vectors such as DNA minicircles or dumbbell-shaped vectors consisting solely of a transcription unit comprising promoter, coding genes and RNA-stabilising sequences, have several advantages such as improved cellular delivery or nuclear diffusion due to small size. Moreover, these small vectors are resistant to exonucleases due to the covalently closed structure, whereas plasmids often harbour single-strand breaks, so-called nicks, triggered by shearing forces. The lack of unnecessary bacterial sequences or resistance proteins eliminates unwanted side effects in the host, and the controlled in vitro synthesis and the option to chemically link fluorophores, cell-penetrating peptides or immune stimulatory peptides to the loop structures, allows easy manipulation of these vectors. Dumbbell-shaped DNA vectors, sometimes called dumbbells, doggybone DNA or closed-ended DNA, comprise (i) a double-stranded DNA core that includes any gene or genes of interest to be delivered as well as regulatory sequences such as promoters, enhancers, nuclear localisation signals, transcriptional termination or polyadenylation signals, and (ii) single-stranded loop structures closing the ends on both sides. As described above transgenic silencing in plasmids is frequent. DNA minicircles lacking extragenic spacers between the 5′ and 3′ ends of the transgene expression cassette were shown to allow sustained transgene expression in mice. When compared with minicircles, dumbbell-shaped vectors can be an order of magnitude smaller in molecular weight, in particular those for the expression of small non-coding RNA. WO2012/032114 discloses a DNA expression construct comprising a dumbbell-shaped circular vector which maintains expression for seven days post injection into melanomas. As opposed to any other genetic vectors, dumbbells can be featured with helper functions for targeted delivery, imaging, immune sensing etc. via the loops.

This disclosure relates to novel dumbbell vector conjugates for targeted delivery. We present dumbbell vectors that are, amongst other things, conjugated with tri-antennary N-acetylgalactosamine (GalNAc)3 for targeted delivery into asialoglycoprotein-positive cells including hepatocytes. We further present dumbbell vectors that are conjugated with aptamers, i.e., a CD137 and a prostate-specific membrane antigen (PSMA)-targeting aptamer. Both, (GalNAc)3 and the aptamers are non-covalently linked to an enlarged dumbbell loop structure via complementary base pairing.

### STATEMENTS OF INVENTION

According to a first aspect of the invention, there is provided a trans-splicing RNA (tsRNA) molecule comprising:
a) at least one, but preferably multiple, binding domain(s) specific for at least a part of a gene that associates with or is a biomarker for a cell, or a diseased cell, to be treated; and
b) nucleic acids encoding at least one or more expressible:
   (i) suicide protein or a protein that is a component of a suicide system; or
   (ii) fluorescent protein, luciferase or another reporter protein; or
   (iii) therapeutic protein; and
c) at least one splice signal; and
d) at least one safety domain specific for the splice signal within the trans-splicing RNA.

In a preferred embodiment of the invention said binding domain comprises a binding site comprising at least 25, more preferably 35, even more preferably 45, and most preferably 55 or more consecutive unstructured nucleotides (nt) having no internal binding and/or self-complementary sequences; and said binding domain, when of a length of 44 nt or longer, has at least one, or a plurality of, mismatch nucleotide(s) with respect to said gene.

In a preferred embodiment of the invention any one or more, including all, of parts b) i-iii may be present in said tsRNA, including any combination thereof.

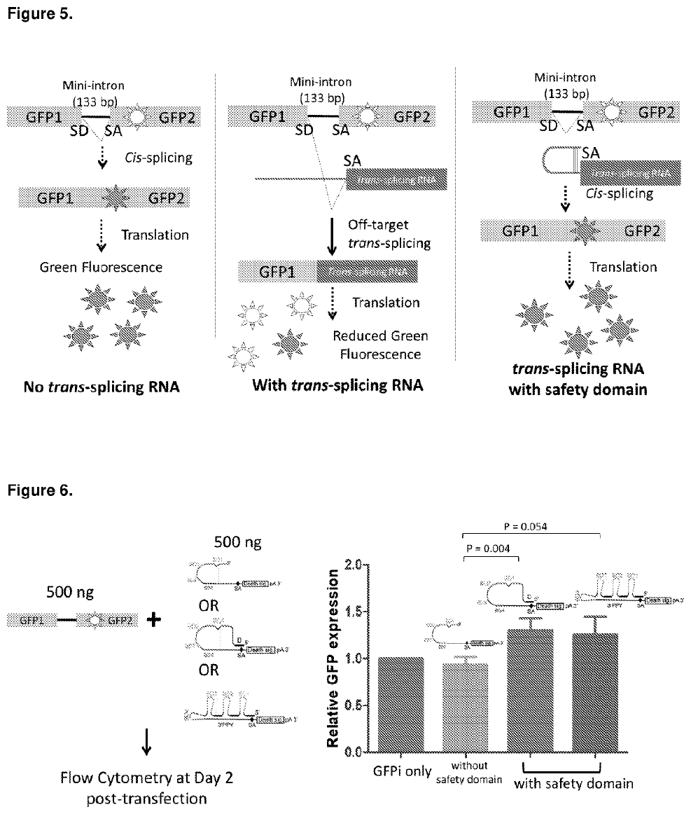

Reference herein to a safety domain is reference to an antisense binding domain specific for a splice site in the splice signal of the trans-splicing RNA. The safety domain is arranged to prevent off-target trans-splicing as the binding domain has to bind to its target to release the safety domain from the splice signal in order to enable on-target trans-splicing. This is best seen with reference to FIG. 5.

In a preferred embodiment of the invention said safety domain is either a linear sequence of nucleic acids or a folded sequence of nucleic acids comprising one or more folds, herein referred to as a continuous safety domain and a segmented safety domain, respectively.

Accordingly in other preferred embodiments of this invention are the novel safety domains, i.e., antisense sequences, with respect to the trans-splicing RNAs, that are complementary to, at least a part of the tsRNA's splice signal domain, or its splice donor (SD) and polypyrimidine tract (PPY). These safety domains prevent off-target trans-splicing as any of the target binding domains has to bind to its target first to release the safety domain from the SD and PPY in order to enable on-target trans-splicing. Two designs of safety domains were invented and tested: 1. a continuous safety domain and 2. a segmented safety domain the latter of which was disrupted by the various target binding domains.

Reference herein to a trans-splicing RNA molecule is reference to a molecule that interacts with a target precursor mRNA molecule and mediates a trans-splicing event to generate a novel chimeric mRNA that can be processed in the cell to yield a protein product.

Reference herein to a gene that associates with or is a biomarker for a target cell or a diseased cell to be treated is reference to a gene that is characteristic of said cell or said diseased cell and so is exclusively or preferentially present or expressed in/by said cell or when said disease occurs.

Reference herein to a housekeeping gene is reference to a gene that does not associate with a disease and one that is ubiquitously and abundantly expressed in any cell.

A novel aspect or embodiment of the invention is the generation of multi-targeting trans-splicing-based suicide RNAs targeting disease-specific and/or housekeeping gene derived pre-mRNAs. As opposed to the disease-specific pre-mRNA biomarkers, pre-mRNAs derived from housekeeping genes are constitutively expressed in all cell types. The housekeeping genes include but are not limited to genes involved in gene expression, metabolism, cellular structure, cellular surfaces, signaling, and others. This novel design applies to target cells in which the disease-specific biomarkers are very limited by numbers and/or the level of expression. In such a scenario, trans-splicing towards a housekeeping sequence can trigger basal expression of the death signal but without yet killing the cells. Only additional trans-splicing towards a disease or cell type-specific pre-mRNA biomarker will elevate the expression of the death signal above the threshold that finally kills the cell.

Reference herein to a protein that is a component of a suicide system is reference to a protein that interacts with, directly or indirectly, at least one other molecule to trigger or result in death of a cell in which said protein is expressed.

Those skilled in the art will appreciate that the tsRNA has nucleotides complementary to a gene with which it is to bind and because it is RNA it will include the nucleotides adenosine, guanosine, cytidine or uridine and the respective bases adenine, guanine, cytosine, and uracil or known chemical modifications of the same.

As those skilled in the art know, RNA is a chain of nucleotides, but unlike DNA, it is often found in nature as a single strand folded onto itself due to the presence of self-complementary sequences that allow parts of the RNA to fold and pair with itself to form a highly structured molecule. Thus, reference herein to an unstructured state is reference to a state within said binding sites where the sequence of RNA nucleotides exists in an unfolded chain. This chain may be curved or bent but it is not folded; thus there is no internal binding or self-complementary sequences.

As an alternative, a further way of describing an unstructured state is where said binding site comprises a sequence predicted not to fold into a stable minimum free energy secondary structure (Gibbs free energy of RNA secondary structure formation ΔG≥0 kcal/mol) or is at least less structured than the average of possible binding domains (ΔG>ΔGaverage). While RNAfold indicates such structures as open circle, mfold would not give any result for structures with ΔG≥0 kcal/mol.

In preferred embodiments of the invention, said binding domain is not fully complementary to the target gene, or pre-mRNA, and so said binding domain does not form perfect duplexes with the target gene and, usually, is not longer than 200 bp, most usually not longer than 100 bp.

In certain embodiments of the invention said binding domain, including said binding site, comprise a sequence of nucleotides selected from the list comprising or consisting of: 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 7 3, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300 or more nucleotides.

In yet further certain embodiments of the invention said binding domain comprises a sequence of nucleotides that is at least 50%, 55%, 60%, 65%, 70%, 75%, 80%, 85%, 90%, 91%, 92%, 93%, 94%, 95%, 96%, 97%, 98%, 99% or 100% complementary to said part of said gene that associates with or is a biomarker for a disease to be treated, or, that alternatively is expressed in any cell. Ideally, the mismatches in said binding domain are positioned in way to avoid any stretches of 44 nt or longer that are perfectly complementary to the target, including or excluding said binding sites, and ideally at least 5 nucleotides from the 5′ or 3′ end. We think these features most likely act by suppressing antisense effects including A-to-I editing that might result from the formation of long double-stranded RNA in the nucleus. These features appear to be particularly beneficial in 3′ER.

In certain embodiments the binding domain, particularly when it is short i.e., less than 100 nucleotides including the binding site, is located adjacent a spacer sequence that helps to maintain the unstructured nature of the binding domain and so in this embodiment the tsRNA also includes a spacer sequence, known to those skilled in the art, adjacent said binding domain.

Those skilled in the art will appreciate that said binding domain can be designed to bind to any pre-mRNA including any biomarker of any disease and, providing it has the recited features, efficient trans-splicing will occur. Thus, the suicide gene therapy constructs, or sequences described herein can easily be reprogrammed to target alternative diseases or diseased tissue simply by exchanging the target binding domain(s).

In yet further certain embodiments of the invention said trans-splicing RNA molecule comprises a plurality of said binding domains which are complementary to the same or different parts of a gene that associates with or is a biomarker for a disease to be treated. Where more than one binding domain is for the same part of the gene, we have found it significantly improves specific on-target trans-splicing. Where more than one binding domain is for a different part of the gene, or even a different gene, we have found it enhances trans-splicing activity and improves the trans-splicing phenotype.

In certain further embodiments said tsRNA is either 5′ or 3′ tsRNA.

Accordingly, in one aspect or embodiment of the invention there is provided a cytotoxic tsRNA, ideally based on a suicide gene therapy approach.

In yet a further preferred embodiment said tsRNA comprises a tri-antennary N-acetylgalactosamine (GalNAc)3, for targeted delivery into asialoglycoprotein-positive cells, including hepatocytes.

In yet a further preferred embodiment said tsRNA is a dumbbell RNA. More preferably said dumbbell RNA comprises a tri-antennary N-acetylgalactosamine (GalNAc)3, for targeted delivery into asialoglycoprotein-positive cells, including hepatocytes.

In yet a further preferred embodiment said tsRNA is encoded by a dumbbell-shaped DNA vector. More preferably said tsRNA is a dumbbell RNA which comprises a tri-antennary N-acetylgalactosamine (GalNAc)3, for targeted delivery into asialoglycoprotein-positive cells, including hepatocytes.

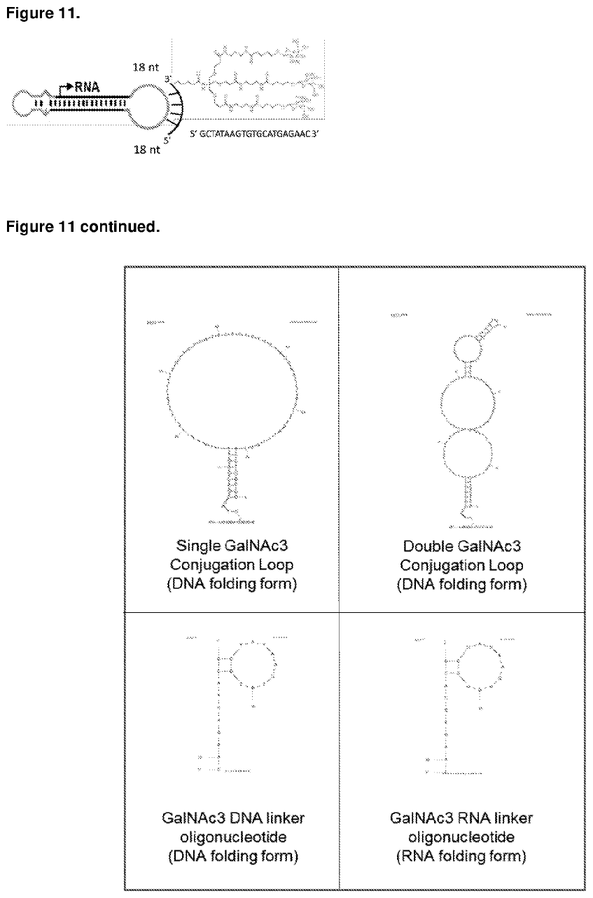

In another embodiment of this invention are dumbbell-shaped delivery vectors featured with (GalNAc)3 residues for targeted delivery into hepatocytes, wherein they comprise at least one residue but, ideally, multiple residues. GalNAc residues are frequently used to deliver oligomeric nucleic acids including antisense oligodeoxyribonucleotides (ASOs) or small interfering RNAS (siRNAs) into hepatocytes. We have for the first-time provided gene expression vectors, i.e., dumbbell vectors, with (GalNAc)3 residues. Therefore (GalNAc)3 labelled antisense DNA or RNA oligos were hybridized to produce an extended complementary dumbbell loop via complementary base pairing (FIG. 11). In the RNA but not in the DNA conjugates, the endogenous enzyme RNaseH can cleave the RNA within the RNA-DNA heteroduplex and thereby release the (GalNAc)3 residues inside the cells facilitating nuclear dumbbell diffusion.

In another embodiment of this invention are trans-splicing-based suicide RNAs targeting hepatoblastoma-derived cells harbouring target binding domains which are complementary to alpha-feto protein (AFP), Vascular endothelium growth factor (VEGF), Γ-glutamyl transferase (GGT), Hepatocellular carcinoma associated protein 2 (HCCA2), Transforming growth factor beta 1 (TGF-β1), cluster of differentiation 24 (CD24), Cyclin D1 (CCND1), Glypican 3 (GPC3) and Telomerase reverse transcriptase (TERT).

Additionally or alternatively, said tsRNA comprises at least one aptamer, such as, CD137 and/or a prostate-specific membrane antigen (PSMA)-targeting aptamer. Preferably, either or both, (GalNAc)3 and said aptamer(s) is/are non-covalently or covalently linked to the tsRNA.

Additionally or alternatively, said tsRNA is a dumbbell RNA which comprises at least one aptamer, such as, CD137 and/or a protatate-specific membrane antigen (PSMA)-targeting aptamer. Preferably, either or both, (GalNAc)3 and said aptamer(s) is/are non-covalently linked to an enlarged dumbbell loop structure via complementary base pairing.

Additionally or alternatively, said tsRNA is encoded by a dumbbell-shaped DNA vector which encodes/comprises at least one aptamer, such as, CD137 and/or a prostate-specific membrane antigen (PSMA)-targeting aptamer. Preferably, either or both, (GalNAc)3 and said aptamer(s) is/are non-covalently linked to an enlarged dumbbell loop structure via complementary base pairing.

In another embodiment of this invention are dumbbell-shaped delivery vectors featured with at least one aptamer, e.g., a CD137 binding aptamer, for targeted delivery into CD137+ cells. In another embodiment of this invention are dumbbell-shaped delivery vectors featured with at least one aptCD137-2 residue, but, ideally, multiple residues.

In another embodiment of this invention are dumbbell-shaped delivery vectors featured with at least one prostate-specific membrane antigen (PSMA) binding aptamer for targeted delivery into prostate cancer cells. These dumbbells deliver suicide RNAs comprise prostate-specific antigen (PSA) pre-mRNA targeting domains.

In yet a further embodiment of the invention said tsRNA comprises or is characterized by any one or more of the sequences herein described, including any combination thereof.

According to a second aspect of the invention, there is provided a trans-splicing RNA (tsRNA) molecule comprising:
a) at least one binding domain specific for at least a part of a gene that associates with or is a biomarker for a cell, or a diseased cell, to be treated; and
b) at least one binding domain specific for at least a part of a gene that is ubiquitously expressed in any cell; and
c) nucleic acids encoding at least one or more expressible:
   (i) suicide protein or a protein that is a component of a suicide system; or
   (ii) fluorescent protein, luciferase or other reporter protein; or
   (iii) therapeutic protein; and
d) at least one splice signal; and
e) at least one safety domain specific for the splice signal within the trans-splicing RNA; wherein
said gene that is ubiquitously expressed in any cell is a housekeeping gene.

Ideally, said binding domain comprises a binding site comprising at least 25, more preferably 35, even more preferably 45, and most preferably 55 or more consecutive unstructured nucleotides (nt) having no internal binding and/or self-complementary sequences and said binding domain, when of a length of 44 nt or longer, has at least one, or a plurality of, mismatch nucleotide(s) with respect to said gene.

In a preferred embodiment of the invention any one or more, including all, of parts c) i-iii may be present in said tsRNA.

Our work shows, where part b (i) of the first aspect of the invention or part c (i) of the second first aspect of the invention is deployed, that these optimised tsRNAs efficiently triggered the death of cells containing same, such as, hepatoblastoma-derived cells, HBV-positive cells, CD137-positive cells, nasopharyngeal cancer cells, epidermal cells, basal cells, hair follicle cells, and senescent cells.

In another embodiment of this invention are trans-splicing-based suicide RNAs targeting HBV-positive cells harbouring target binding domains which are complementary to HBV pre-genomic RNA (pgRNA), AFP and GPC3. As the expression of HBV pgRNA is lower in cell culture than the expression of other HCC-specific targets, HBV-targeting RNAs are less active at lower concentration. However, HBV RNA targets are expressed at much higher levels in HBV-infected cells in vivo.

In another embodiment of this invention are trans-splicing-based suicide RNAs targeting nasopharyngeal cancer cells harbouring target binding domains which are complementary to various oncogenic pre-mRNAs as described herein.

In another embodiment of this invention are trans-splicing-based suicide RNAs targeting EBV-positive cells harbouring target binding domains which are complementary to Epstein-Barr virus pre-mRNAs (i.e. BZLF1, EBNA-3B, LMP1 and LMP2A).

In another embodiment of this invention are trans-splicing RNAs targeting epidermal cells harbouring target binding domains which are complementary to Keratin 1 (KRT1), Keratin 2 (KRT2), Keratin 10 (KRT10), Keratin 14 (KRT14), Caspase-14 precursor (CASP14), Neuroblast differentiation-associated protein 2 (AHNAK2). Another embodiment of this invention are trans-splicing RNAs targeting basal cells harbouring target binding domains which are complementary to Keratin 15 (KRT15), Collagen 17A1 (COL17A1), Tumour protein 73 (TP73). In another embodiment of this invention are trans-splicing RNAs targeting hair follicle cells harbouring target binding domains which are complementary to Homeobox C13 (HOXC13), Fibroblast growth factor 7 (FGF-7). In another embodiment of this invention are trans-splicing RNAs targeting senescent cells harbouring target binding domains which are complementary to Forkhead Box O4 (FOXO4) and cyclin-dependent kinase inhibitor 2A (p16). All these trans-splicing RNAs were either featured with a GFP gene for imaging or the HSVtk gene to trigger cell death. The sequences were delivered using dumbbell vectors into the epidermis of domestic pigs following non-invasive topical application.

In another embodiment of this invention are trans-splicing-based suicide RNAs encoding death signals other than the HSVtk, such as CYLD Lysine 63 Deubiquitinase (CYLD), tumor necrosis factor-like weak inducer of apoptosis (TWEAK), Tumor necrosis factor-related apoptosis-inducing ligand (TRAIL) and Tumor necrosis factor alpha (TNF-α) pre-mRNA.

Thus, spliceosome-mediated RNA trans-splicing represents a promising therapeutic strategy to trigger cell death in suicide gene therapy approaches.

In another preferred embodiment of this invention, said biomarker is any single biomarker or combination of biomarkers selected from the group comprising cancer biomarkers, including HCC biomarkers alpha-feto protein (AFP), Vascular endothelium growth factor (VEGF), γ-glutamyl transferase (GGT), Hepatocellular carcinoma associated protein 2 (HCCA2), Transforming growth factor beta 1 (TGF-β1), cluster of differentiation 24 (CD24), Cyclin D1 (CCND1), Glypican 3 (GPC3), Telomerase reverse transcriptase (TERT), α-L-fucosidase (AFU), CD19, CD34, CD44, CD49E, CD51, CD105, Collagen type XV alpha 1 (COL15A1), C-X-C motif chemokine receptor 4 (CXCR4), Denticleless E3 ubiquitin protein ligase homolog (DTL), Epithelial cell adhesion molecule (EPCAM), Golgi protein 73 (GP73), G protein signaling modulator 2 (GPSM2), Hepatocyte growth factor (HGF), Heat shock protein 70 (HSP70), Insulin like growth factor 2 (IGF2), Immunoglobulin superfamily member 3 precursor (IGSF3), Integrin Subunit Alpha 6 (ITGA6), Kell blood group glycoprotein (KEL), KIT Proto-Oncogene, Receptor Tyrosine Kinase (KIT), Minichromosome Maintenance Complex Component 3 (MCM3), Minichromosome Maintenance Complex Component 7 (MCM7), PDZ Binding Kinase (PBK), DNA Polymerase Delta 1, Catalytic Subunit (POLD1), Protein Regulator Of Cytokinesis 1 (PRC1), SRY-Box Transcription Factor 17 (SOX17), Spermatogenesis-associated serine-rich protein 2 (SPATS2), Translocon-associated protein subunit beta (SSR2), Stathmin 1 (STMN1), Thrombomodulin (THBD), ZW10 Interacting Kinetochore Protein (ZWINT), HBV-derived RNA including HBV pgRNA, Epstein-Barr virus derived RNA and pre-mRNAs including BamHI Z Epstein-Barr virus replication activator (BZLF1), Epstein-Barr virus nuclear antigen 3B (EBNA-3B), Latent membrane protein 1 (LMP1), and Latent membrane protein 2A (LMP2A), epidermal cell markers including Keratin 1 (KRT1), Keratin 2 (KRT2), Keratin 10 (KRT10), Keratin 14 (KRT14), Caspase-14 precursor (CASP14), Neuroblast differentiation-associated protein 2 (AHNAK2), basal cell markers including Keratin 15 (KRT15), Collagen 17A1 (COL17A1), Tumour protein 73 (TP73), hair follicle cell markers including Homeobox C13 (HOXC13) and Fibroblast growth factor 7 (FGF-7), senescent cell markers including Forkhead Box 04 (FOXO4) and cyclin-dependent kinase inhibitor 2A (p16), the stratum corneum markers Kallikrein related peptidase 5 (KLK5), Small proline-rich protein 4 (SPRR4), and Arachidonate 12-lipoxygenase (ALOX12B), the Stratum spinosum (Upper epidermal layers) markers HOP homeobox (HOPX) and Kallikrein 9 (KLK9), the Stratum granulosum markers Filaggrin (FLG) and Premature ovarian failure 1B protein (POF1B), the Melanocyte markers Melan-A (MLANA) and Tyrosinase (TYR), the Langerhans cell markers CD1A and CD207, the fibroblast markers Periostin (POSTN) and Phospholipase C-eta-2 protein (PLCH2), the basal cell carcinaoma markers Glioma 1 (Gll1), Glioma 2 (GII2), Forkhead box protein (FOXM1), Forkhead box protein (FOXO3A), Desmoglein 2 (DSG2) and C3b, the basal cell carcinoma recurrence markers Cyclooxygenase (COX-2), Ezrin (EZR), CD25, Maspin, Glioma 3 (GII3) and Gremlin1.

In other embodiments said disease is cancer or a viral infection or a bacterial infection or an acquired genetic disease caused by mutations triggered by transposable elements, radiation, chemicals, or unknown triggers.

In the first instance said cancer is selected from the group comprising: hepatocellular carcinoma (HCC), cervical cancer, vaginal cancer, vulvar cancer, penile cancer, skin cancers, melanoma including malignant melanoma, squamous-cell carcinoma, basal-cell carcinoma, Merkel cell carcinoma, lung cancer, cell bladder cancer, breast cancer, colon or rectal cancer, anal cancer, endometrial cancer, kidney cancer, leukemia, acute myelogenous or myeloid leukemia (AML), acute lymphoblastic leukemia (ALL), chronic myeloid leukemia (CML), chronic myelogenous or myeloid leukemia (CML), hairy cell leukemia (HCL), T-cell prolymphocytic leukemia (P-TLL), large granular lymphocytic leukemia, adult T-cell leukemia, lymphoma, myeloma, non-Hodgkin lymphoma, pancreatic cancer, prostate cancer, thyroid cancer, nasopharyngeal cancer, mouth or throat cancer, oropharyngeal cancers, stomach cancer, brain tumours, bone cancer, and stem cell cancers and, indeed other cancers that would benefit from the treatment disclosed herein.

In the second instance said viral infection is selected from the group comprising: Papillomaviruses, human papillomavirus type 16, human papillomavirus type 18, retroviruses, lentiviruses, herpes viruses, adenovirus, adeno-associated virus, Flu virus, Hepatitis virus, Hepatitis B virus (HBV), Hepatitis C virus (HCV), Epstein-Barr virus (EBV), human T-cell lymphotropic virus (HTLV), human immunodeficiency virus (HIV), human immunodeficiency virus type 1 (HIV-1), and human immunodeficiency virus type 2 (HIV-2), and others.

In the third instance said bacterial infection is selected from the group comprising: Bartonella henselae, Francisella tularensis, Listeria monocytogenes, salmonella species, Salmonella typhi, Brucella species, Legionella species. Mycobacteria species, Mycobacterium tunberculosis, Nocardia species, Rhodococcus species, Yersinia species, Neisseria meningitides and others.

In the last instance said acquired genetic disease is selected from the group comprising: Neurofibromatosis 1 and 2, Mc Cune Albright, Duchenne muscular dystrophy (DMD), Epidermolysis bullosa, Fanconi A and C, Philadelphia chromosome, Hemophilia A and B, cystic fibrosis, Muckle Wells syndrome, lipoprotein lipase deficiency, B-thalassemia, Gaucher Disease types I to III—GBA gene, Ornithine transcarbamylase (OTC) deficiency—OTC, Phenylketonuria (PKU)—PAH gene, Aspartylglucosaminuria—AGA gene, Alpha-1 anti trypsin deficiency (AATD)—SERPINA1, pyruvate dehydrogenase complex deficiency, and others.

In a further preferred embodiment of the invention, tsRNA is provided with a helper function for targeted delivery by the use of a peptide or carbohydrate such as GalNAc3, ideally more than one GalNAc3 is conjugated to the dumbbell tsRNA, ideally, by attaching two GalNAc3 residues via RNA linker oligonucleotides to a HSVtk expressing dumbbell vector, preferably in the region of the circular ends.

In an alternative aspect there is provided a medicament comprising said tsRNA according to the invention and, optionally where part b (i) of the first aspect of the invention or part c (i) of the second aspect of the invention is deployed, at least one further component of said suicide system effective to trigger death of a cell expressing said trans-spliced RNA.

In an alternative aspect there is provided a pharmaceutical composition comprising said tsRNA according to the invention and, optionally where part b (i) of the first aspect of the invention or part c (i) of the second aspect of the invention is deployed, at least one further component of said suicide system effective to trigger death of a cell expressing said trans-spliced RNA; and a carrier suitable for human or veterinary use.

In the afore optional instance said further component may be, e.g., ganciclovir, although other known co-component suicide systems for cell death may be used. Examples are cytosine deaminase-5-fluorocytosine, cytochrome P450-ifosfamide, cytochrome P450-cyclophosphamide, and nitroreductase-5-[aziridin-1-yl]-2,4-dinitrobenzamide.

In an alternative aspect there is provided a cell containing said tsRNA or a vector, ideally a dumbbell-shaped DNA expression vector, comprising or consisting of said tsRNA.

In a further aspect there is provided method of killing a cell comprising transfecting, lipofecting, transducing, electroporating, nucleofecting or transforming said cell with tsRNA, or a vector containing the tsRNA, according to the invention and, optionally where part b (i) of the first aspect of the invention or part c (i) of the second aspect of the invention is deployed, exposing said cell to at least one other component of said suicide system effective to trigger death of a cell expressing said trans-spliced RNA.

In a further aspect there is provided a method of treating a disease comprising transfecting, lipofecting, transducing, electroporating, nucleofecting or transforming a diseased cell with tsRNA, or a vector containing the tsRNA, according to the invention ex vivo or in vivo and, optionally where part b (i) of the first aspect of the invention or part c (i) of the second aspect of the invention is deployed, exposing said cell to at least one other component of said suicide system effective to trigger death of a cell expressing said trans-spliced RNA.

In a further aspect there is provided a method of targeting a diseased cell comprising topical application (including a cream, a gel, a foam, a lotion, ointment or aerosol), intranasal application, alveolar application, systemic application, oral application, intravenous application, intramuscular application, subcutaneous application, cutaneous application, intraperitoneal application, or injection into a tumor with tsRNA, or a vector containing the tsRNA, according to the invention in vivo and, and, optionally where part b (i) of the first aspect of the invention or part c (i) of the second aspect of the invention is deployed, exposing said cell to other components of said suicide system effective to kill said cell.

In a certain methods of the invention said cell is a virally transformed cell. Typically the cell is transformed with a virus selected from the group comprising: Papillomaviruses, human papillomavirus type 16, human papillomavirus type 18, retroviruses, lentiviruses, herpes viruses, adenovirus, adeno-associated virus, Flu virus, Hepatitis virus, Hepatitis B virus (HBV), Hepatitis C virus (HCV), Epstein-Barr virus (EBV), human T-cell lymphotropic virus (HTLV), human immunodeficiency virus (HIV), human immunodeficiency virus type 1 (HIV-1), and human immunodeficiency virus type 2 (HIV-2).

In yet other methods of the invention said cell is a cancer cell such as a hepatocellular carcinoma (HCC) cell, cervical cancer cell, vaginal cancer cell, vulvar cancer cell, penile cancer cell, skin cancer cell, melanoma cell including malignant melanoma cell, squamous-cell carcinoma cell, basal-cell carcinoma cell, Merkel cell carcinoma cell, lung cancer cell, cell bladder cancer cell, breast cancer cell, colon or rectal cancer cell, anal cancer cell, endometrial cancer cell, kidney cancer cell, leukemia cell, acute myelogenous or myeloid leukemia (AML) cell, acute lymphoblastic leukemia (ALL) cell, chronic myeloid leukemia (CML) cell, chronic myelogenous or myeloid leukemia (CML) cell, hairy cell leukemia (HCL) cell, T-cell prolymphocytic leukemia (P-TLL) cell, large granular lymphocytic leukemia cell, adult T-cell leukemia cell, lymphoma cell, myeloma cell, non-Hodgkin lymphoma cell, pancreatic cancer cell, prostate cancer cell, thyroid cancer cell, nasopharyngeal cancer cell, mouth or throat cancer cell, oropharyngeal cancer cell, stomach cancer cell, brain tumour cell, bone cancer cell, and stem cell cancer cell. Ideally said cell is mammalian and most typically human.

According to a third aspect, or a further embodiment, of the invention there is provided a dumbbell-shaped DNA expression vector comprising:
a) one or more linear or hairpin-shaped transcription cassettes each comprising a nucleotide sequence encoding a nucleic acid molecule to be expressed;
b) two single-stranded DNA loops;
c) operably linked to said transcription cassette a minimal transcription promoter nucleotide sequence and a transcription terminator sequence;
d) a nucleotide sequence comprising a DNA sequence that functions as nuclear targeting sequence;
e) a nucleotide sequence comprising a spliceable intron; and
f) a residue or helper function for targeted delivery, covalently or non-covalently linked to at least one of the loops.

In a further preferred embodiment of the invention said vector comprises at least one internal loop domain. Preferably, said loop domain comprises an abasic site or a nucleotide mismatch.

In a preferred embodiment of the invention said abasic site comprises one or more apurinic/apyrimidinic abasic sites.

In a preferred embodiment of the invention said nucleotide mismatch comprises a tetrahydrofuran-based mimic of an abasic site.

In a preferred embodiment of the invention said nucleic acid molecule to be expressed encodes a therapeutic protein or peptide.

In a preferred embodiment of the invention said therapeutic protein is Cas9, Cas9n, hSpCas9 or hSpCas9n.

In a preferred embodiment of the invention said therapeutic protein or peptide triggers a death signal.

Examples of proteins or peptides that trigger a cellular death signal are known in the art. For example, bacterial toxins such as the cholera toxin or the diphtheria toxin, alpha toxin, anthrax toxin, exotoxin, pertussis toxin, shiga toxin, shiga-like toxin etc are known to induce cell death. Furthermore, apoptotic signals/proteins such as Fas, TNF, caspases (initiator caspases, caspase 2,8,9,10,11,12, and effector caspases, caspase 3,6,7) etc. In addition, enzymes that are able to convert a non-toxic drug into a toxic component: e.g. the herpes simplex virus thymidine kinase (HSVtk) converts the rather non-toxic drug ganciclovir (GCV) into the toxic triphosphate (HSVtk/GCV system). A further example is the Escherichia coli purine nucleoside phosphorylase (PNP)/fludarabine suicide gene system.

In a further preferred embodiment of the invention said therapeutic protein or peptide is the HSVtk.

In an alternative preferred embodiment of the invention said expressed nucleic acid molecule is a therapeutic nucleic acid molecule.

In a preferred embodiment of the invention said therapeutic nucleic acid is a siRNA or shRNA.

In an alternative preferred embodiment of the invention said therapeutic nucleic acid molecule is an antisense RNA oligonucleotide or antisense miRNA.

In a further preferred embodiment of the invention said therapeutic nucleic acid molecule is a miRNA.

In a further preferred embodiment of the invention said therapeutic nucleic acid molecule is a trans-splicing RNA.

In a further preferred embodiment of the invention said therapeutic nucleic acid molecule is a guide RNA, single-guide RNA, crRNA, or tracrRNA.

In an alternative preferred embodiment of the invention said therapeutic nucleic acid molecule is a pre-mRNA or mRNA.

In a further preferred embodiment of the invention said minimal transcription promoter is derived from an RNA polymerase III promoter.

In a preferred embodiment of the invention said RNA 5 polymerase III promoter is a U6 promoter.

In an alternative preferred embodiment of the invention said RNA polymerase III promoter is a H1 promoter.

In an alternative preferred embodiment of the invention said RNA polymerase III promoter is a minimal H1 (mH1) promoter.

In a further alternative preferred embodiment of the invention said RNA polymerase III promoter is a modified mH1 promoter that includes a restriction endonuclease cleavage site and/or an inverted polymerase III transcriptional terminator.

In a further preferred embodiment of the invention said minimal transcription promoter is derived from an RNA polymerase II promoter.

In a preferred embodiment of the invention said RNA polymerase II promoter is a CMV promoter comprising the nucleotide sequence set forth in SEQ ID NO: 240.

In a preferred embodiment of the invention said transcription terminator nucleotide sequence is a RNA polymerase II or RNA polymerase III termination sequence.

In a preferred embodiment of the invention said RNA polymerase III termination sequence comprises one or more motifs comprising the nucleotide sequence TTTTT.

In a preferred embodiment of the invention said DNA nuclear targeting sequence comprises the nucleotide sequence set forth in SEQ ID NO: 1.

In a further preferred embodiment of the invention said enhancer nucleotide sequence comprises the nucleotide sequence set forth in SEQ ID NO: 1 (full length enhancer: 30 fSV40enh).

In a further preferred embodiment of the invention said vector comprises the intron nucleotide sequence set forth in SEQ ID NO: 241.

In a further preferred embodiment of the invention, said residue or helper function for targeted delivery is a carbohydrate.

In a further preferred embodiment of the invention, said residue or helper function for targeted delivery is a (GalNAc)₃ residue.

In an alternative preferred embodiment of the invention, said residue or helper function for targeted delivery is an aptamer.

In a further preferred embodiment of the invention, said aptamer is a CD137 or a PSMA binding aptamer.

In a further preferred embodiment of the invention, said aptamers are non-covalently bound towards a dumbbell loop via complementary base pairing using the sequence set forth in SEQ ID NO: 242.

In a further preferred embodiment of the invention, said enlarged dumbbell loop comprises any of the sequences set forth in SEQ ID NO: 5 and SEQ ID NO: 6.

In a further preferred embodiment of the invention, said residue or helper function for targeted delivery is an antibody.

In a further preferred embodiment of the invention, said residue or helper function for targeted delivery is a CD137-binding antibody.

In a further preferred embodiment of the invention, said residue or helper function for targeted delivery is a peptide or carbohydrate such as GalNAc3, ideally more than one GalNAc3 is conjugated to the dumbbell vector.

In a further preferred embodiment of the invention, said residue or helper function for targeted delivery is a cell penetrating peptide.

In a further preferred embodiment of the invention said vector further encodes a detectable marker.

In a preferred embodiment of the invention said detectable marker 5 is a fluorescence marker.

In a preferred embodiment of the invention said fluorescence marker is a fluorescent reporter protein.

The analysis of promoter activity in a tissue can be conveniently monitored by fusing a promoter to a nucleic acid that encodes a “reporter” protein or polypeptide. Examples are well known in the art and include enzymes such as @ glucuronidase. Reporters that are proteinaceous fluorophores are also known in the art. Green fluorescent protein, GFP, is a spontaneously fluorescent protein isolated from coelenterates, such as the Pacific jellyfish, Aequoria victoria. Its role is to transduce, by energy transfer, the blue chemiluminescence of another protein, aequorin, into green fluorescent light. GFP can function as a protein tag, as it tolerates N- and C-terminal fusions to a broad variety of proteins many of which have been shown to retain native function. Most often it is used in the form of enhanced GFP in which codon usage is adapted to the human code. Other proteinaceous fluorophores include yellow, red and blue fluorescent proteins. These are commercially available from, for example, Clontech (www.clontech.com). A yet further example is firefly luciferase.

In a preferred embodiment of the invention said nucleotide sequence with homology, to a part of a mammalian genome, for targeted delivery is implemented into the double-stranded DNA part of the dumbbell vector.

In an alternative preferred embodiment of the invention said nucleotide sequence with homology, to a part of a mammalian genome, for targeted delivery is implemented into the single-stranded loop of the dumbbell vector.

According to a further aspect of the invention there is provided a pharmaceutical composition comprising a dumbbell-shaped vector according to the invention and a suitable carrier.

The dumbbell-shaped vector compositions of the present invention are administered in pharmaceutically acceptable preparations. Such preparations may routinely contain pharmaceutically acceptable concentrations of salt, buffering agents, preservatives, compatible carriers and supplementary therapeutic agents. The dumbbell shaped vector compositions of the invention can be administered by any conventional route, including injection or by gradual infusion over time. The administration may, for example, intravenous, intraperitoneal, intramuscular, intracavity, subcutaneous, transdermal, oral, topical, intratracheal, nasal, intravaginal or trans-epithelial. Alternatively, the dumbbell-shaped vector or vector composition of this invention is delivered by physical methods including but not limited to liquid jet-injection, microinjection, microneedles, powder particle injection, gold particle injection, gene gun, electroporation or hydrodynamic injection.

The dumbbell-shaped vector compositions of the invention are administered in effective amounts. An “effective amount” is that amount of the dumbbell-shaped vector that alone, or together with further doses, produces the desired response. In the case of treating a disease, the desired response is inhibiting the progression of the disease. This may involve only slowing the progression of the disease temporarily, although more preferably, it involves halting the progression of the disease permanently. This can be monitored by routine methods. Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The dumbbell-shaped vector compositions used in the foregoing methods preferably are sterile and contain an effective amount of dumbbell-shaped vector according to the invention for producing the desired response in a unit of weight or volume suitable for administration to a patient. The doses of vector administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits. Other protocols for the administration of vector compositions will be known to one of ordinary skill in the art, in which the dose amount, schedule of injections, sites of injections, mode of administration and the like vary from the foregoing. The administration of compositions to mammals other than humans, (e.g. for testing purposes or veterinary therapeutic purposes), is carried out under substantially the same conditions as described above. A subject, as used herein, is a mammal, preferably a human, and including a non-human primate, cow, horse, pig, sheep, goat, dog, cat or rodent.

When administered, the dumbbell-shaped vector compositions of the invention are applied in pharmaceutically acceptable amounts and in pharmaceutically acceptable compositions. The term “pharmaceutically acceptable” means a non-toxic amount that does not interfere with the effectiveness of the biological activity of the active agent. Such preparations may routinely contain salts, buffering agents, preservatives, compatible carriers, and optionally other therapeutic agents' (e.g. those typically used in the treatment of the specific disease indication). When used in medicine, the salts should be pharmaceutically acceptable, but non-pharmaceutically acceptable salts may conveniently be used to prepare pharmaceutically acceptable salts thereof and are not excluded from the scope of the invention. Such pharmacologically and pharmaceutically acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically acceptable salts can be prepared as alkaline metal or alkaline earth salts, such as sodium, potassium or calcium salts.

The pharmaceutical compositions containing dumbbell-shaped vectors according to the invention may contain suitable buffering agents, including: acetic acid in a salt; citric acid in a salt; boric acid in a salt; and phosphoric acid in a salt. The pharmaceutical compositions also may contain, optionally, suitable preservatives, such as: benzalkonium chloride; chlorobutanol; parabens and thimerosal.

The dumbbell-shaped vector compositions may conveniently be presented in unit dosage form and may be prepared by any of the methods well-known in the art of pharmacy. All methods include the step of bringing the dumbbell-shaped vector into association with one or more accessory ingredients. Compositions containing vectors according to the invention may be administered as aerosols and inhaled. Compositions suitable for parenteral administration conveniently comprise a sterile aqueous or non-aqueous preparation of the vectors, which is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally acceptable diluent or solvent, for example, as a solution in 1, 3-butanediol. Among the acceptable solvents that may be employed are water, Ringer's solution, and isotonic sodium chloride solution. In addition, sterile, fixed oils are conventionally employed as a solvent or suspending medium.

For this purpose, any bland fixed oil may be employed including synthetic mono- or diglycerides.

In addition, fatty acids such as oleic acid may be used in the preparation of injectables. Carrier formulation suitable for oral, subcutaneous, 5 intravenous, intramuscular, etc. administrations can be found in Remington's Pharmaceutical Sciences, Mack Publishing Co., Easton, PA.

Preferred features of each aspect of the invention may be as described in connection with any of the other aspects of the invention.

Throughout the description and claims of this specification, the singular encompasses the plural unless the context otherwise requires. In particular, where the indefinite article is used, the specification is to be understood as contemplating plurality as well as singularity, unless the context requires otherwise.

In the claims which follow and in the preceding description of the invention, except where the context requires otherwise due to express language or necessary implication, the word “comprises”, or variations such as “comprises” or “comprising” is used in an inclusive sense, i.e., to specify the presence of the stated features but not to preclude the presence or addition of further features in various embodiments of the invention.

All references, including any patent or patent application, cited in this specification are hereby incorporated by reference. No admission is made that any reference constitutes prior art. Further, no admission is made that any of the prior art constitutes part of the common general knowledge in the art.

Other features of the present invention will become apparent from the following examples. Generally speaking, the invention extends to any novel one, or any novel combination, of the features disclosed in this specification (including the accompanying claims and drawings). Thus, features, integers, characteristics, compounds, or chemical moieties described in conjunction with a particular aspect, embodiment or example of the invention are to be understood to be applicable to any other aspect, embodiment or example described herein, unless incompatible therewith.

Moreover, unless stated otherwise, any feature disclosed herein may be replaced by an alternative feature serving the same or a similar purpose.

The present invention will now be described by way of example only with particular reference to the following figures wherein:

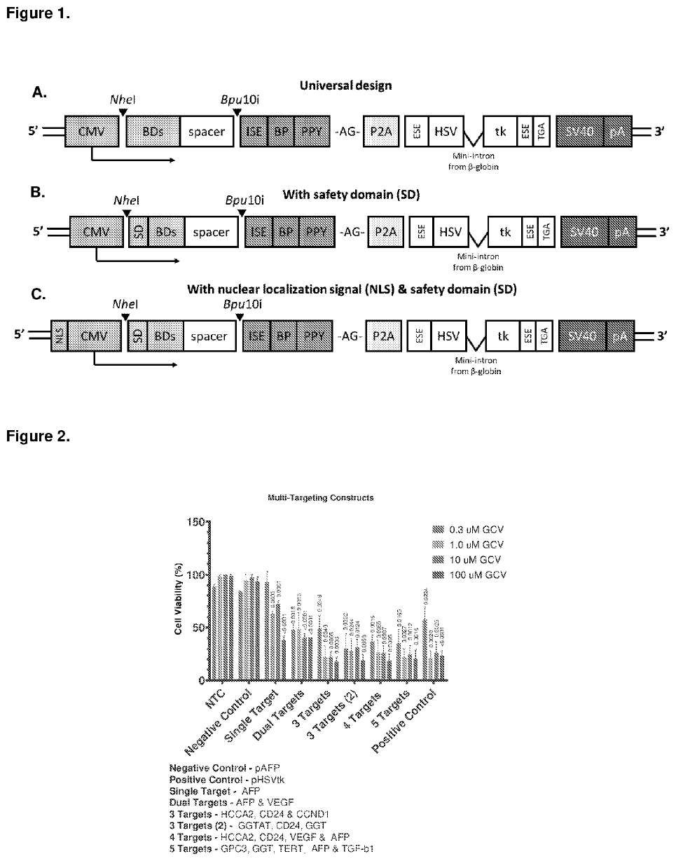

FIG. 1. Shows the design of universal trans-splicing-based suicide RNAs for cell type-specific HSVtk expression and cell death. A) Basic design, B) design including a safety domain, C) design including safety domain and DNA nuclear localization signal. NLS: DNA nuclear localization signal; CMV: CMV promoter; SD: safety domain; BDs: target pre-mRNA binding domains; ISE: intronic splice enhancer; BP: branch point; PPY: polypyrimidine tract; AG: splice acceptor site: P2A: proteolytic cleavage site: ESE: exonic splice enhancer; HSVtk: herpes simplex virus thymidine kinase; TGA: translational stop codon; SV40 pA: simian virus 40 polyadenylation site.

FIG. 2. Shows the multi-targeting suicide RNAs exhibit superior cell death activity on hepatoblastoma-derived human cells compared with single-targeting suicide RNAs even at 300-fold lower GCV doses, alamarBlue cell viability assay.

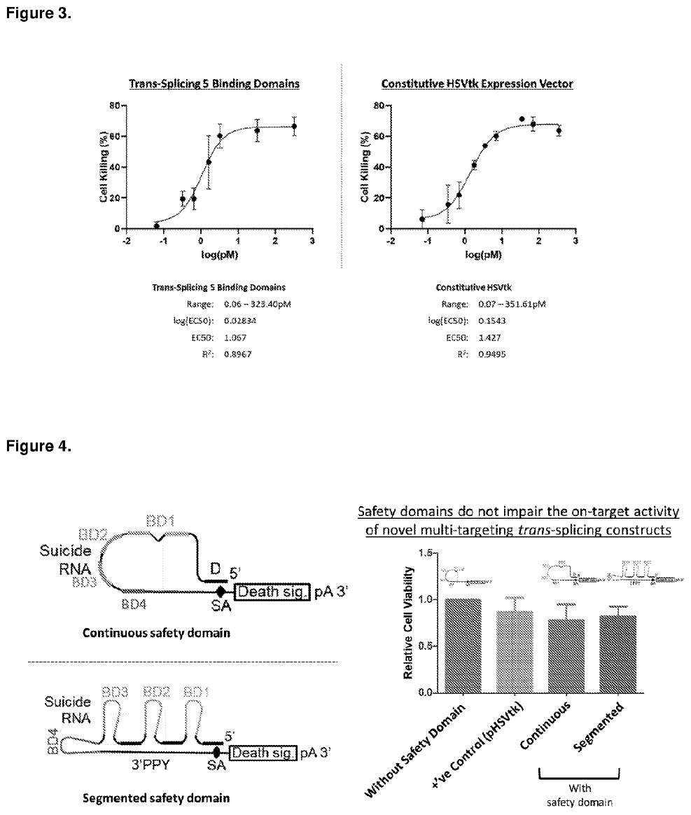

FIG. 3. Shows HCC-targeting suicide RNA (left) exhibits a lower EC₅₀ as compared with the positive control (right), i.e., the constitutively HSVtk expressing vector.

FIG. 4. Shows safety domains do not impair the suicide activity of the trans-splicing-based suicide RNAs. Left: Safety domain design. Continuous safety domain (upper part) and segmented safety domain (lower part). Right: AlamarBlue assay comparing the suicide activity of different vectors.

FIG. 5. Shows a GFP reporter assay to monitor off-target trans-splicing. The GFP gene was disrupted into 2 parts (GFP1 and GFP2) by a functional mini-intron inserted upstream of the chromophore and placing GFP2 out of frame. A, Upon transfection of human cells with the reporter vector, cis-splicing triggers the formation of a functional mature GFP mRNA leading to GFP expression. B, Upon co-transfection of a trans-splicing vector, the trans-splicing RNA may approach the GFP pre-mRNA and off-target trans-splicing may lead to a reduction of the GFP signal. C, A safety domain suppresses off-target trans-splicing and does not lead to a reduction of the GFP signal.

FIG. 6. Shows multi-targeting trans-splicing RNAs featured with safety domains do not exhibit any off-target trans-splicing activity. Left: HepG2 cells were co-transfected with 500 ng of the GFP reporter vector as well as 500 ng of a multi-targeting trans-splicing vector either without or with a continuous or with a segmented safety domain. The vectors without safety domain trigger slightly though not significantly reduced GFP expression. The vectors featured with safety domains exhibit a significantly higher GFP expression indicating the safety domains suppress minor off-target activities triggered by the safety domain-negative vector.

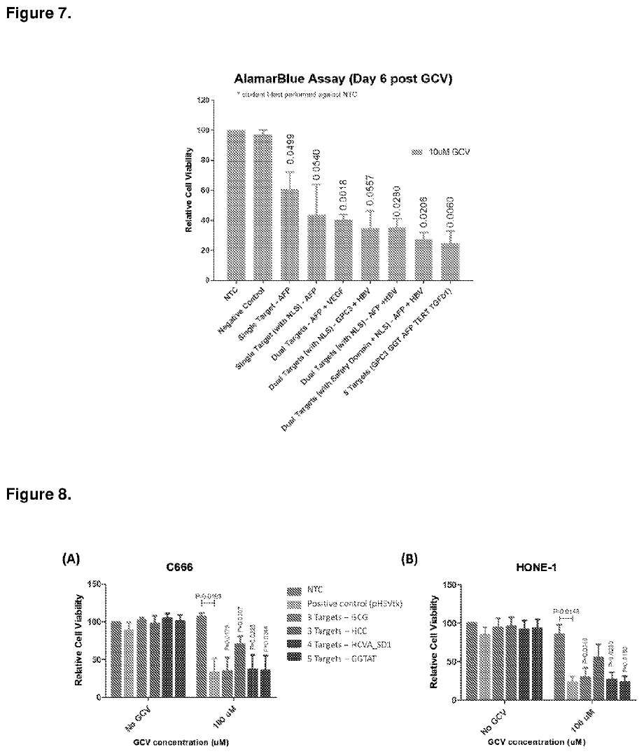

FIG. 7. Shows suicide RNAs featured with binding domains targeting an HCC-specific pre-mRNA (AFP) plus a HBV-specific transcript exhibit comparable cell death activities as compared with HCC-targeting RNAs. However, the HCC/HBV targeting vectors are expected to have a higher specificity for HBV-positive liver cancer cells, using alamarBlue cell viability assay.

FIG. 8. Shows suicide RNAs featured with binding domains targeting cancer-specific pre-mRNA biomarkers effectively kill the nasopharyngeal cancer cells C666 and HONE-1, using alamarBlue cell viability assay.

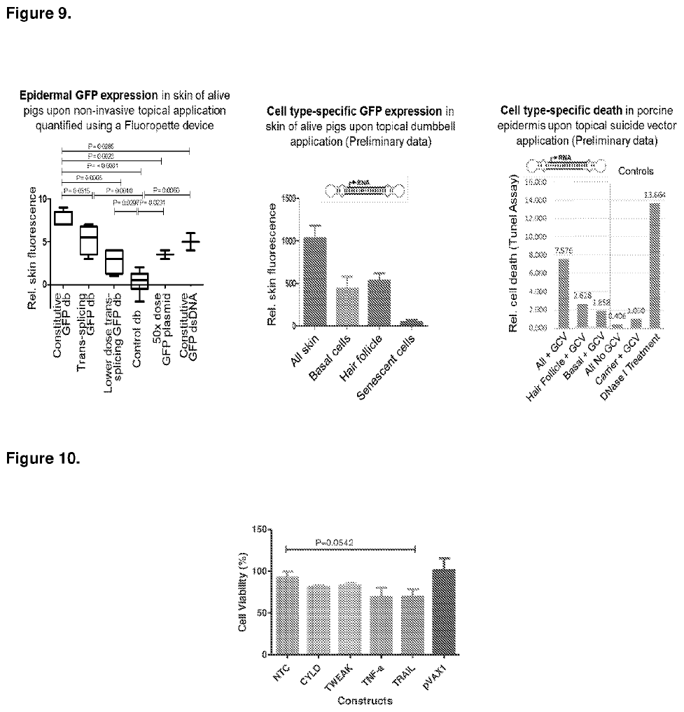

FIG. 9. Shows Dumbbell-shaped trans-splicing vectors were delivered into the porcine epidermis following non-invasive topical application triggering cell type-specific GFP expression (imaging vectors) or cell death (suicide vectors). Left: GFP expression triggered by various types of vectors. Dumbbell, both constitutive or trans-splicing vectors, trigger stronger GFP expression as compared with a 50× higher doses of a constitutively GFP expressing plasmid. Center: Cell type-specific GFP expression triggered by trans-splicing dumbbells. Right: Cell type-specific killing of different epidermal cells triggered by trans-splicing dumbbells.

FIG. 10. Death of HEK293T cells triggered by the expression of apoptosis and necrosis triggers including CYLD Lysine 63 Deubiquitinase (CYLD), tumor necrosis factor-like weak inducer of apoptosis (TWEAK), Tumor necrosis factor-related apoptosis-inducing ligand (TRAIL) and Tumor necrosis factor alpha (TNF-α). NTC: no transfection control; pVAX1: empty cloning vector pVAX1.

FIG. 11. Dumbbell-(GalNAc)3 conjugate & Dumbbell-aptCD137-2 conjugates. (GalNAc)3- or aptCD137-2 labelled antisense oligos were hybridized towards an extended dumbbell loop via complementary base pairing. For optimal hybridization, loops harbouring a 21 nt (GalNAc)3-oligo binding site plus flanking 18 nt spacer sequences were designed.

FIG. 11 Continued. Minimum free energy secondary structures of single and double GalNAc3 conjugation loops (upper panel) and of GalNAc3 DNA and RNA linker oligonucleotides (lower panel) as predicted by mfold's DNA or RNA folding forms.

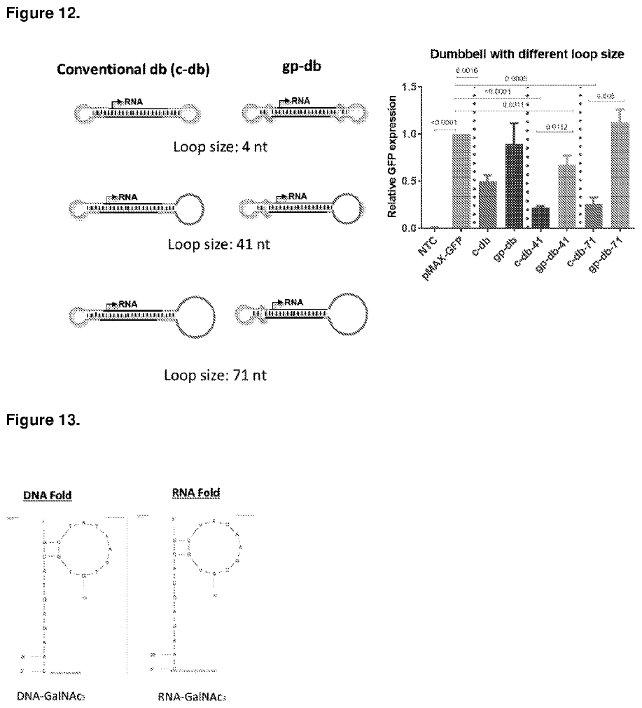

FIG. 12 Shows extended dumbbell loops do not impair dumbbell delivery and gene expression. Dumbbells generated using the gap-primer PCR method (gp-db) exhibit superior activity as compared with conventional dumbbells (c-db) produced using the ELAN method. Increasing the size of conjugation loops (C-loop) does not impair the activity of dumbbell vectors generated using gap-primer PCR (gpPCR). Left hand side, Design of conventional dumbbells (c-db) generated using the ELAN method only and of advanced dumbbells (gp-db) generated using gpPCR, each with various C-loop sizes. Black, double-stranded dumbbell core harbouring the gene of interest; cyan, loops with indicated loop size; magenta, mismatch-triggering abasic position. Right hand side, HepG2 cells were transfected with different dumbbell vectors harbouring different C-loop sizes or the pMAX-GFP vector and MaxGFP expression was monitored 48 hours post transfection using flow cytometry. Mean±SEM (n=3). Significance was tested using the unpaired student's t-test.

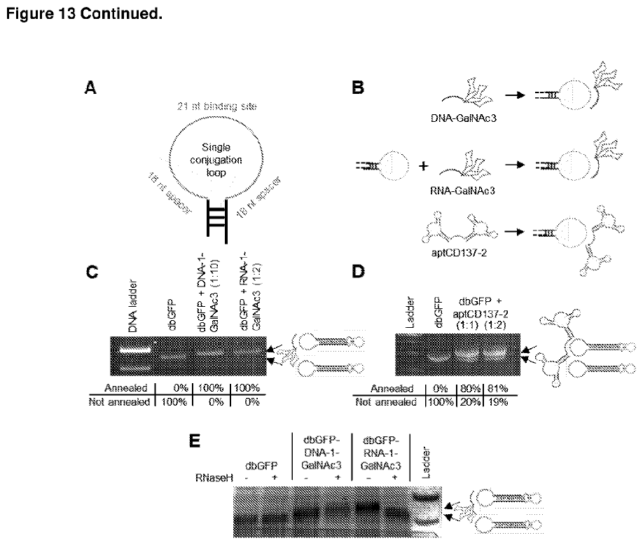

FIG. 13. Shows unstructured DNA or RNA oligos were labelled with (GalNAc)3-residues on the 3′ end.

FIG. 13 Continued Shows Non-covalent linkage of GalNAc3 and aptCD137-2 residues towards dumbbell vector conjugation loops via antisense DNA and RNA oligonucleotides. A, Design of the single conjugation loop comprising a 21 nt conjugation oligonucleotide binding site (blue) and two flanking 18 nt spacers (red). B, Schematic of attaching DNA-GalNAc3, RNA-GalNAc3 and aptCD137-2 residues to the dumbbell conjugation loop. C, Agarose gel electrophoresis analyses of MaxGFP expressing dumbbell vectors (dbGFP) before and after annealing of a single DNA-1-GalNAc3 or RNA-1-GalNAc3 oligonucleotide at 10-(1:10) or 2-fold (1:2) molar excess. Notably, dumbbell-GalNAc3 conjugates were cleaved using the AseI restriction endonuclease and the depicted section of the gel shows the extended conjugation loop only. The percentage of dumbbell-GalNAc3 conjugate formation as quantified using the software imageJ version 1.53u is indicated. D, Agarose gel electrophoresis analyses of MaxGFP expressing dumbbell vectors (dbGFP) before and after annealing of a single aptCD137-2 homodimer using a stochiometric amount (1:1) or 2-fold molar excess (1:2). The percentage of dumbbell-aptSC137-2 conjugate formation as quantified using the software imageJ version 1.53u is indicated. E, Agarose gel electrophoresis analyses of dbGFP-DNA-1- and dbGFP-RNA-1-GalNAc3 conjugates after RNaseH cleavage.

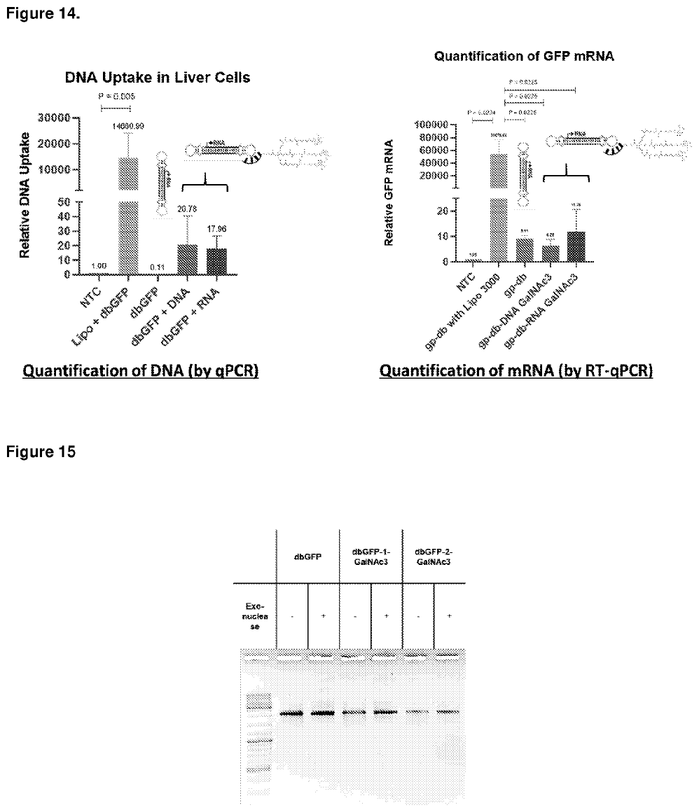

FIG. 14. Shows uptake of GFP-dumbbell-(GalNAc)3-conjugates by HepG2 cells from the culture medium. Left: qPCR-based quantification of the uptake dumbbell DNA. Both, RNA and DNA-oligo conjugates were internalized. Right: RT-qPCR quantification of the expressed GFP mRNA. The RNA-oligo conjugates exhibit higher levels of mRNA expression presumably because RNaseH cleavage of the (GalNAc)3 residues facilitates nuclear dumbbell diffusion.

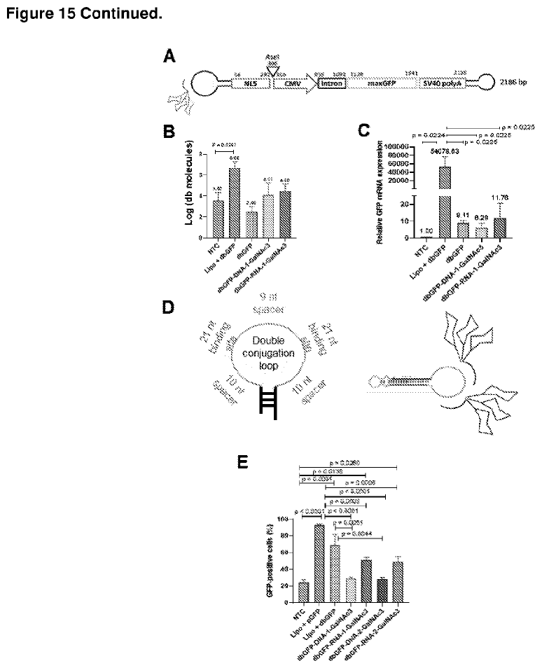

FIG. 15. Dumbbell vectors are exonuclease resistant. Analytical 1% agarose gel analysing MaxGFP expressing dumbbell vectors before and after exonuclease treatment.

FIG. 15 Continued. Uptake and expression of MaxGFP dumbbell-GalNAc3 conjugates by HepG2 cells from culture medium. A, Design of the MaxGFP dumbbell-GalNAc conjugates. B, HepG2 cells well exposed to DNA-linker-(dbGFP-DNA-1-GalNAc3), RNA-linker-(dbGFP-RNA-1-GalNAc3) or non-conjugated (dbGFP) MaxGFP dumbbells or transfected with dbGFP. Intracellular dumbbell DNA was isolated after 24 hours and quantified using qPCR. C, HepG2 cells well exposed to DNA-linker-(dbGFP-DNA-1-GalNAc3), RNA-linker-(dbGFP-RNA-1-GalNAc3) or non-conjugated (dbGFP) MaxGFP dumbbells or transfected with dbGFP. RNA was isolated after 24 hours and quantified using RT-qPCR. D, Design of the double conjugation loop comprising two 21 nt conjugation oligonucleotide binding sites (blue) flanked each by a 10 nt spacer (red) and separated by a 9 nt spacer (red), left panel; schematic of a dumbbell conjugate with two GalNAc3 residues attached to a single conjugation loop. E, Average numbers of GFP=positive cells quantified using flow cytometry analyses of HepG2 cells exposed for 48 hours to dbGFP-GalNAc3 conjugates added to the cell culture medium. NTC: No-transfection control; Lipo+pGFP: Cells transfected with pMaxGFP plasmid and Lipofectamine 3000; Lipo+dbGFP: Cells transfected with MaxGFP expressing dumbbell and Lipofectamine 3000; dbGFP-DNA-1-GalNAc3: MaxGFP expressing dumbbell conjugated with 1 GalNAc3 residue via a DNA linker; dbGFP-RNA-1-GalNAc3: MaxGFP expressing dumbbell conjugated with 1 GalNAc3 residue via a RNA linker: dbGFP-DNA-2-GalNAc3: MaxGFP expressing dumbbell conjugated with 2 GalNAc3 residues via a DNA linker: dbGFP-RNA-2-GalNAc3: MaxGFP expressing dumbbell conjugated with 2 GalNAc3 residues via a RNA linker. Mean±SEM (n=5). Significance was tested using the unpaired student's t-test.

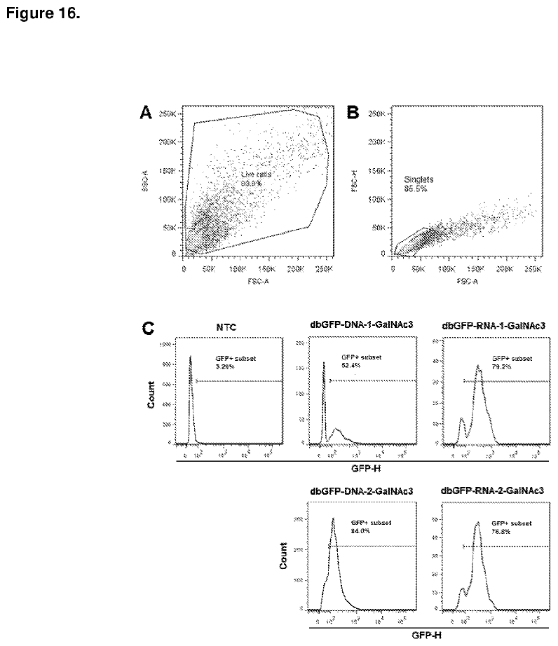

FIG. 16. Flow cytometry analyses of HepG2 cells transfected with MaxGFP dumbbell-GalNAc3-conjugates. A, Representative 2-D scatter plot gating for live HepG2 cells. B, Representative 2-D scatter plot gating for HepG2 cell singlets. C, Representative histograms of flow cytometry analyses of HepG2 cells exposed for 48 hours to dbGFP-GalNAc3 conjugates added to the cell culture medium. NTC: No-transfection control; dbGFP-DNA-1-GalNAc3: MaxGFP expressing dumbbell conjugated with 1 GalNAc3 residue via a DNA linker; dbGFP-RNA-1-GalNAc3: MaxGFP expressing dumbbell conjugated with 1 GalNAc3 residue via a RNA linker: dbGFP-DNA-2-GalNAc3: MaxGFP expressing dumbbell conjugated with 2 GalNAc3 residues via a DNA linker: dbGFP-RNA-2-GalNAc3: MaxGFP expressing dumbbell conjugated with 2 GalNAc3 residues via a RNA linker.

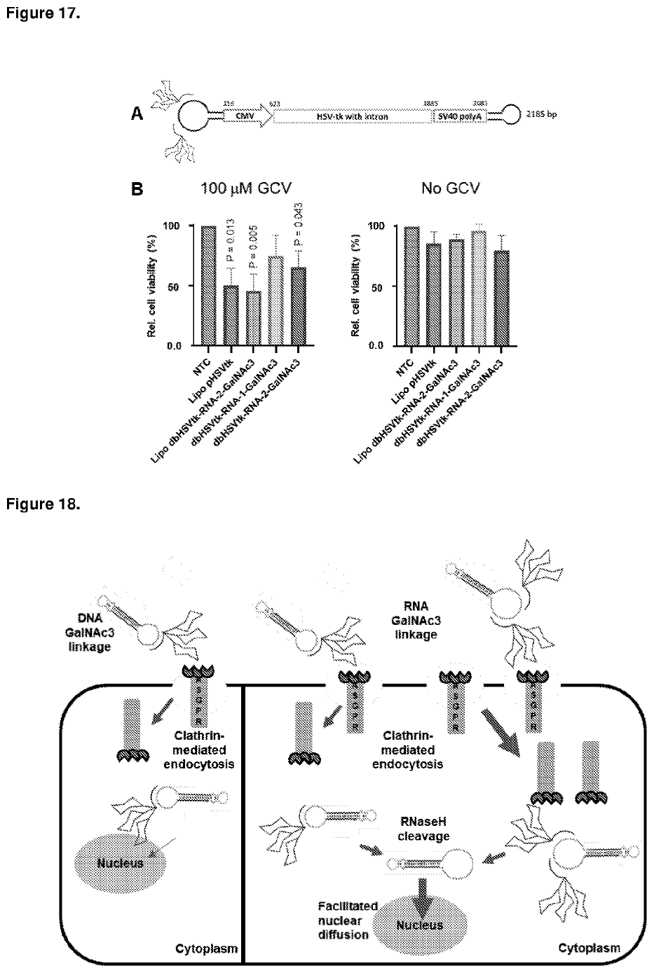

FIG. 17. Death of HepG2 cells triggered by dbHSVtk-GalNAc3 conjugates. A, Design of the HSVtk dumbbell-GalNAc conjugates. B, Death of HepG2 cells triggered by dbHSVtk-GalNAc3 conjugates added to the cell culture medium in the presence (100 μM GCV) or absence (No GCV) of GCV monitored using the alamarBlue cell viability assay. HepG2 cells were either transfected with Lipfectamine 3000 or exposed to HSVtk expressing vectors added to the cell culture medium and cell death was monitored at day 6. NTC: No transfection control; Lipo pHSVtk: HSVtk expressing plasmid delivered via lipofection; Lipo dbHSVtk-RNA-2-GalNAc3: HSVtk expressing dumbbell conjugated with 2 GalNAc3 residues via a RNA linker delivered via lipofection; dbHSVtk-RNA-1-GalNAc3: HSVtk expressing dumbbell conjugated with 1 GalNAc3 residue via a RNA linker added to the medium; dbHSVtk-RNA-2-GalNAc3: HSVtk expressing dumbbell conjugated with 2 GalNAc3 residues via a RNA linker added to the medium. Mean±SEM (n=3). Significance was tested using the unpaired student's t-test.

FIG. 18. Schematic depicting the concept of GalNAc3-mediated cellular uptake and expression of dumbbell vector DNA. Single GalNAc3 conjugates bind towards one asialoglycoprotein receptor (ASGPR) and are internalised by the cell via clathrin-mediated endocytosis. Double GalNAc3 conjugates can bind to two ASGPR receptors which facilitates cellular uptake. RNA-linker—but not DNA-linker-conjugates are cleaved by the endogenous RNaseH resulting in release of the GalNAc3 residues form the dumbbell DNA. Unconjugated dumbbells are less bulky, and exhibit facilitated diffusion through the nuclear pore complex resulting in higher levels of transgene expression.

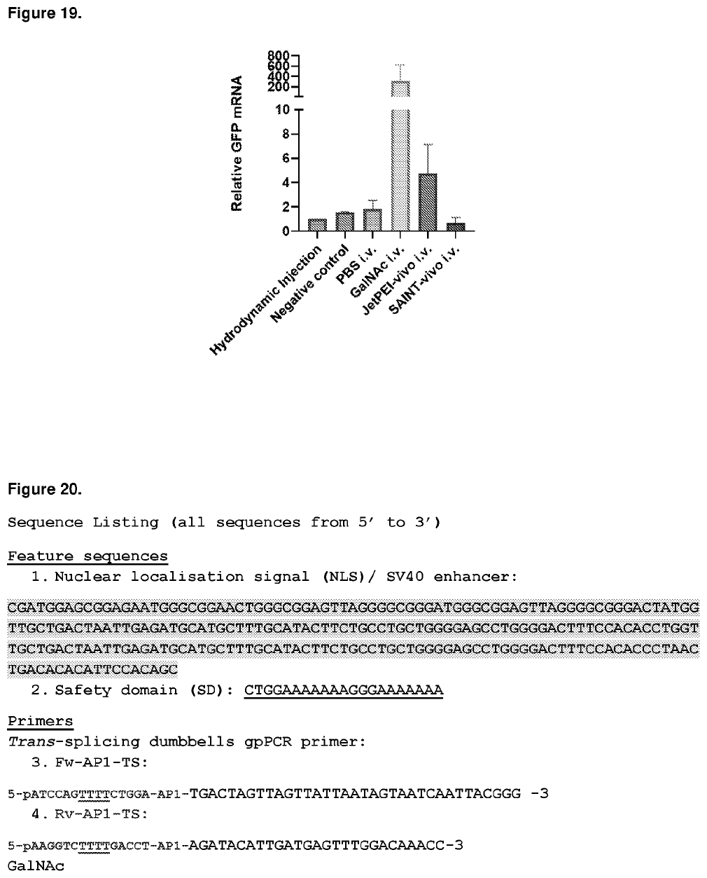

FIG. 19. Shows relative GFP mRNA expression triggered in the livers of mice after intravenous injection of 10 μg GFP expressing dumbbell vectors or dumbbell vector conjugates. Highest GFP mRNA levels were detected after injecting GFP dumbbell-GalNAc3 conjugates. Hydrodynamic injection: Hydrodynamic injection of GFP expressing dumbbell vector; Negative control: non-injected mice; PBS i.v.: PBS injected mice; GalNAc i.v.: mice injected iv with GFP dumbbell-GalNAc3 conjugates; JetPEI-vivo i.v.: mice injected iv with GFP dumbbell-in vivo-JetPEI LNPs; SAINT-vivo i.v.: mice injected iv with GFP dumbbell-SAINT-vivo LNPs. Mean±SEM (n=4).

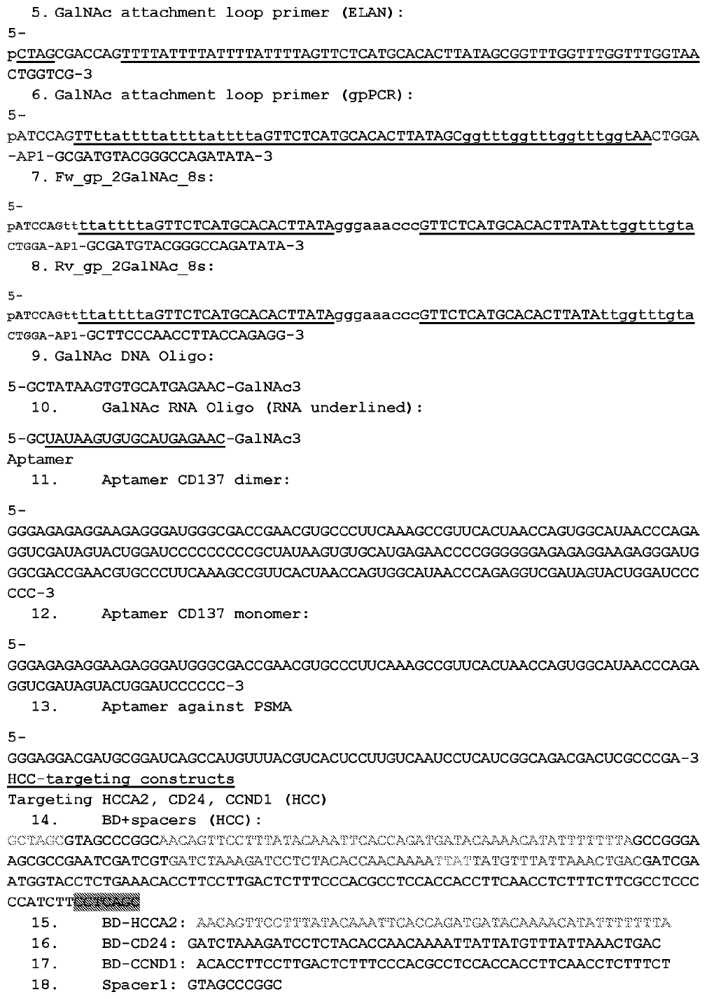

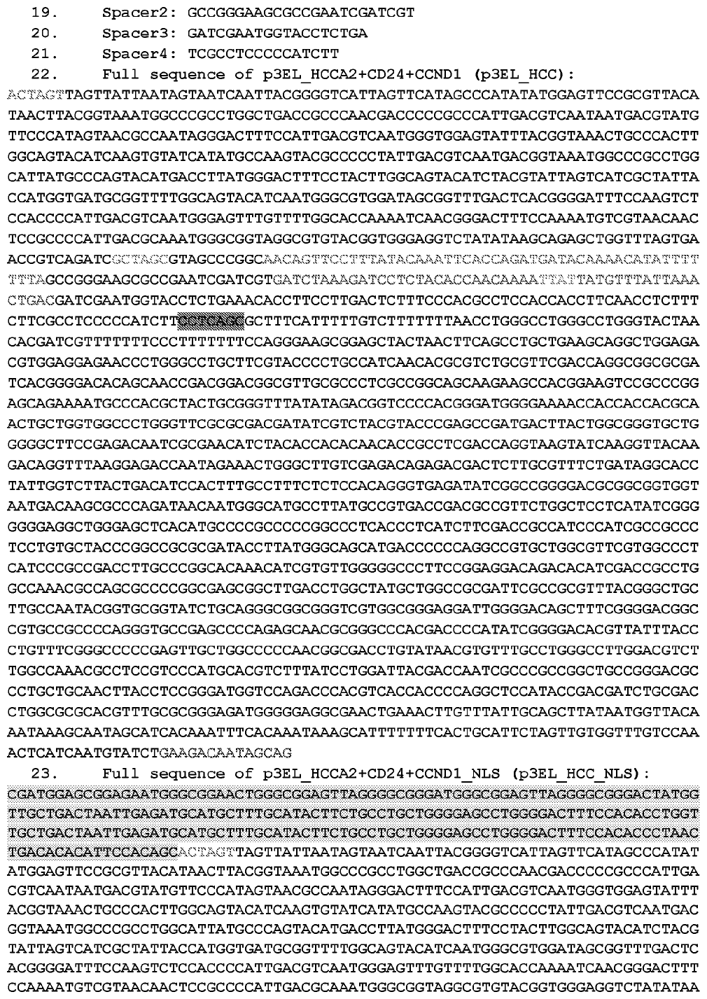

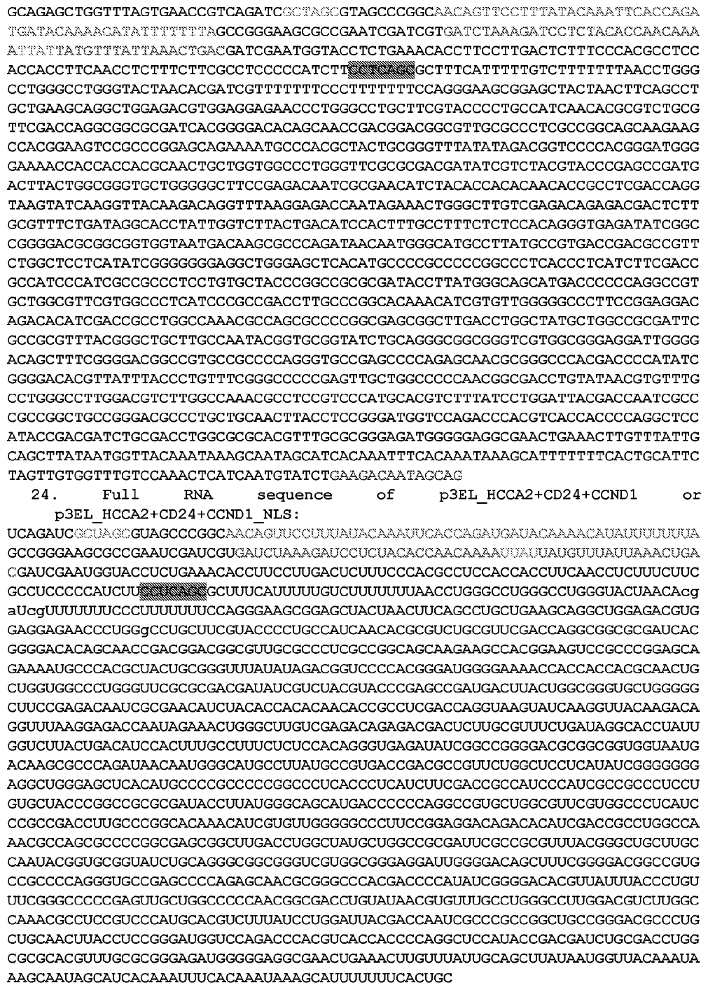

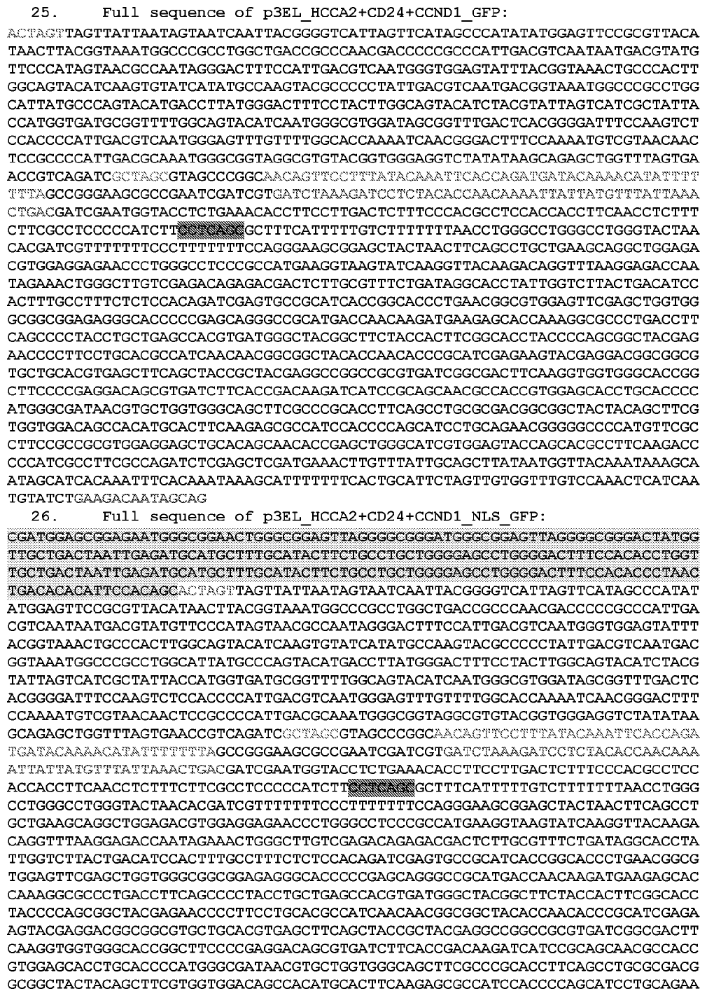

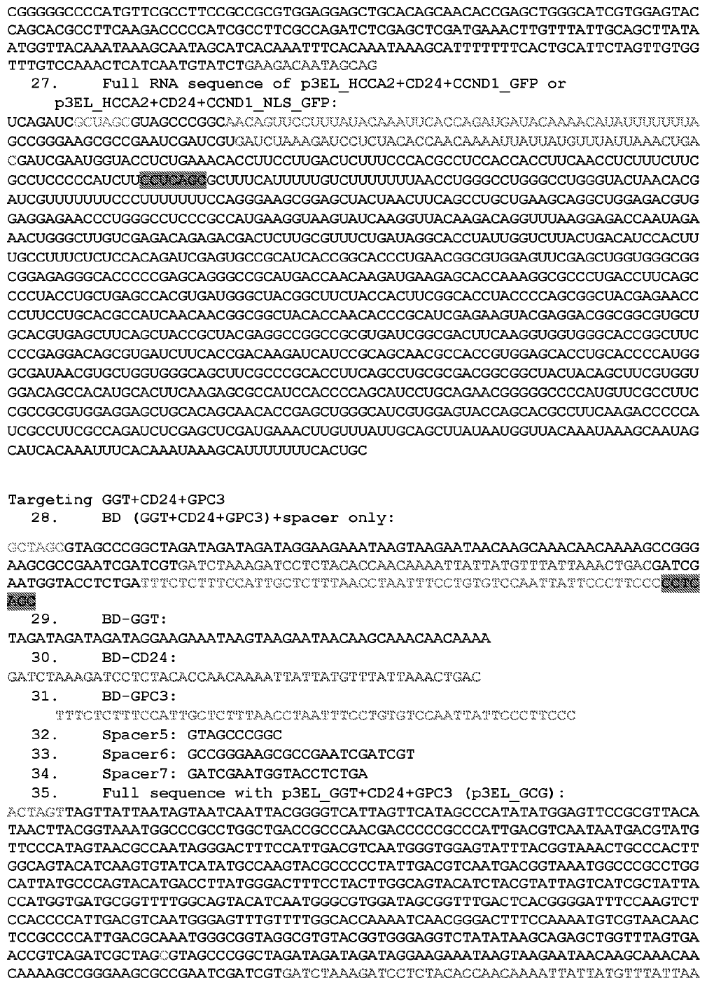

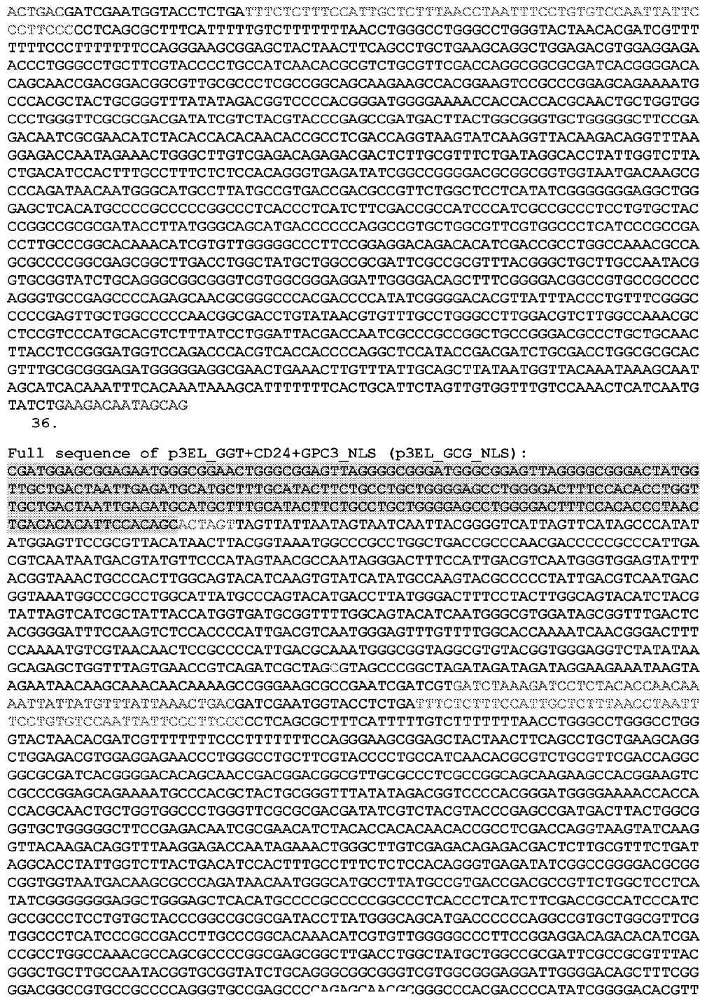

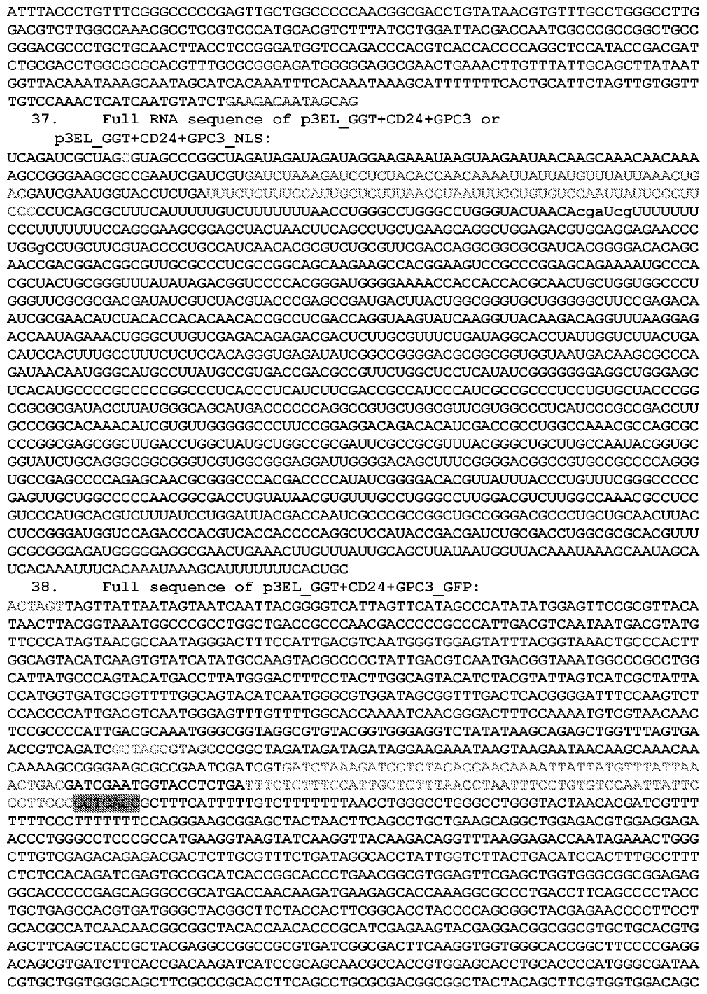

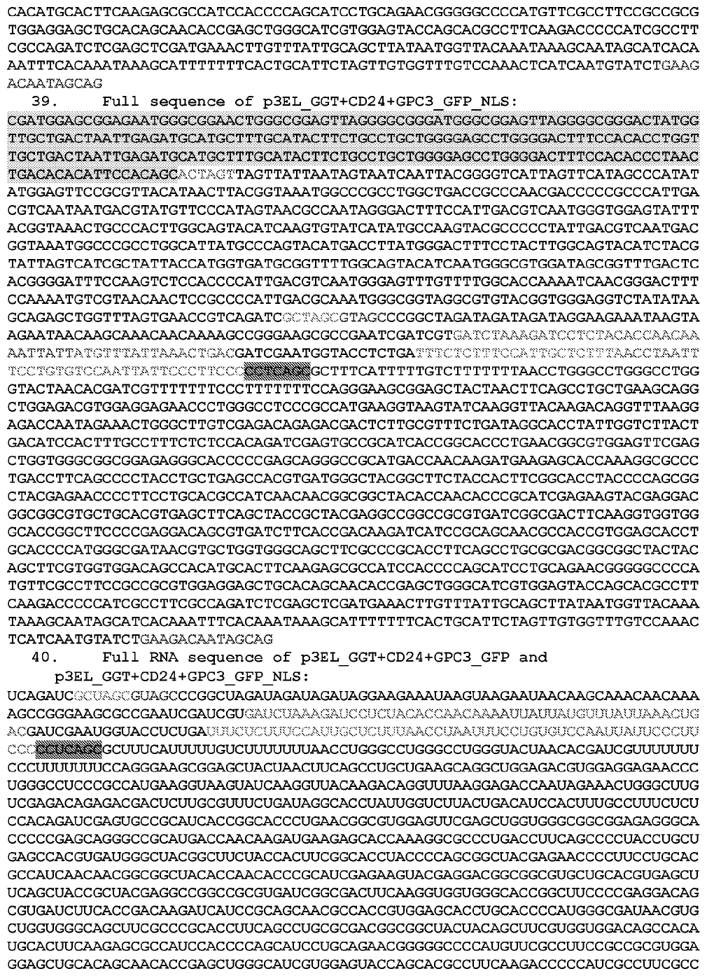

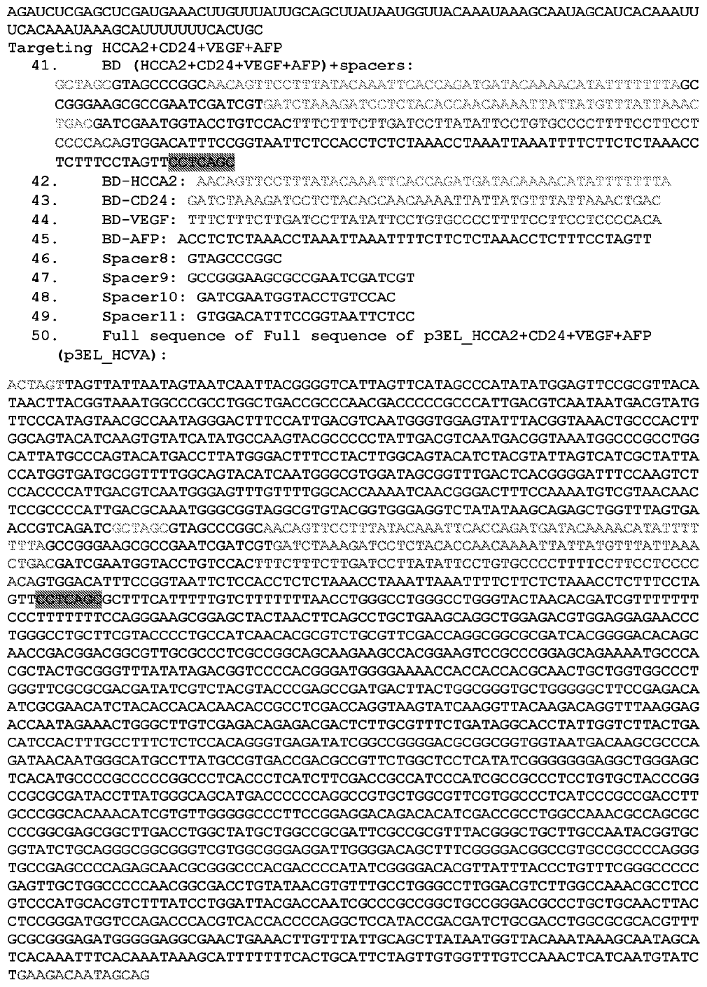

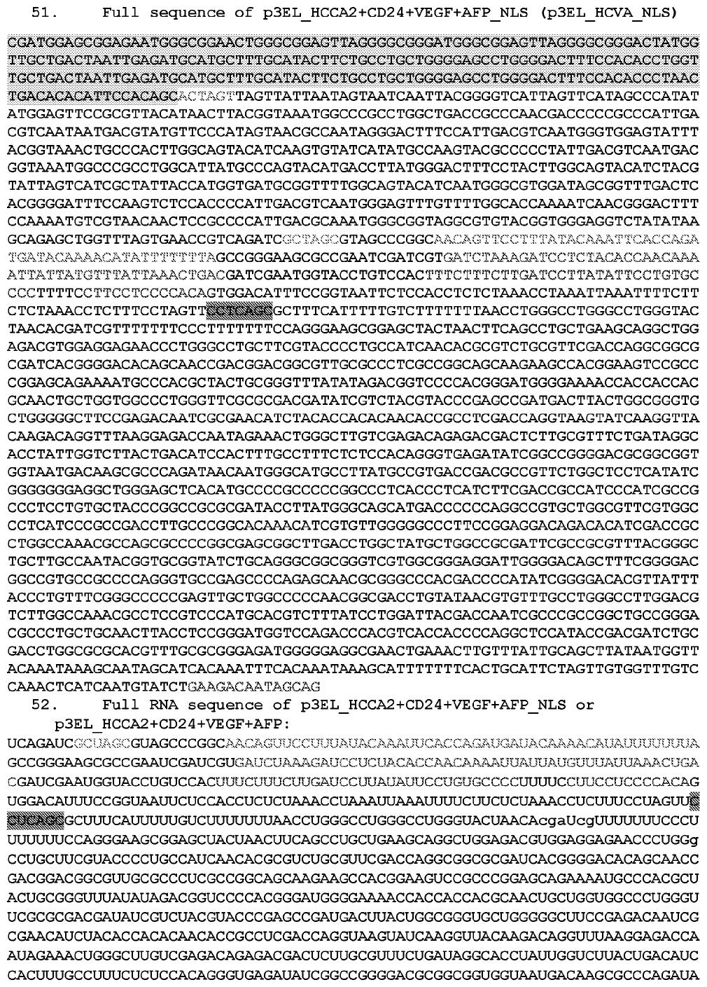

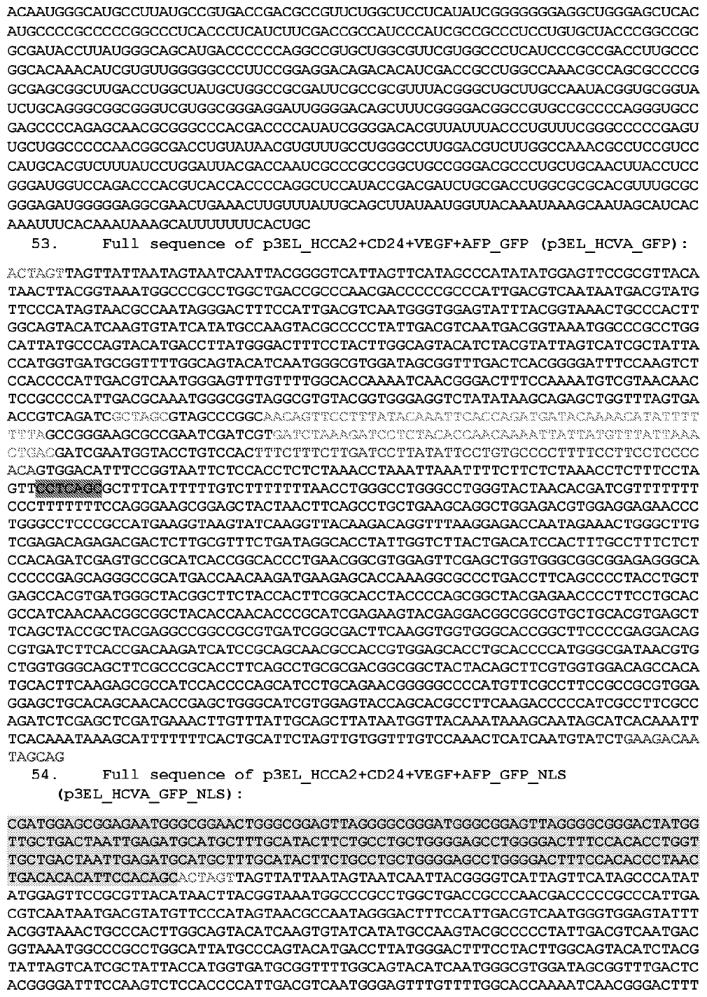

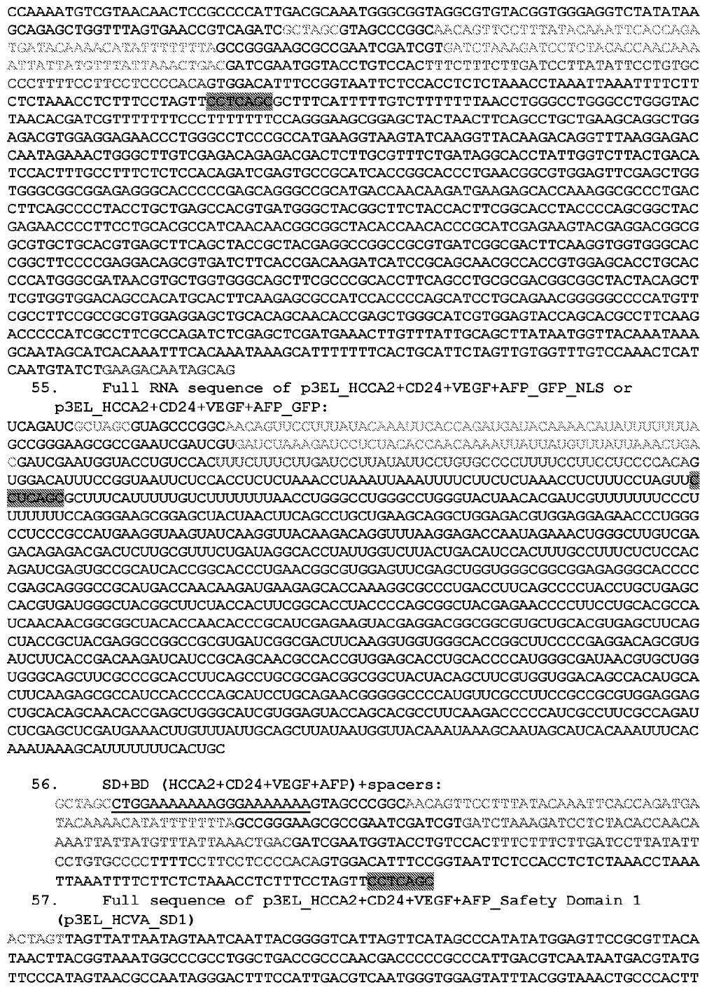

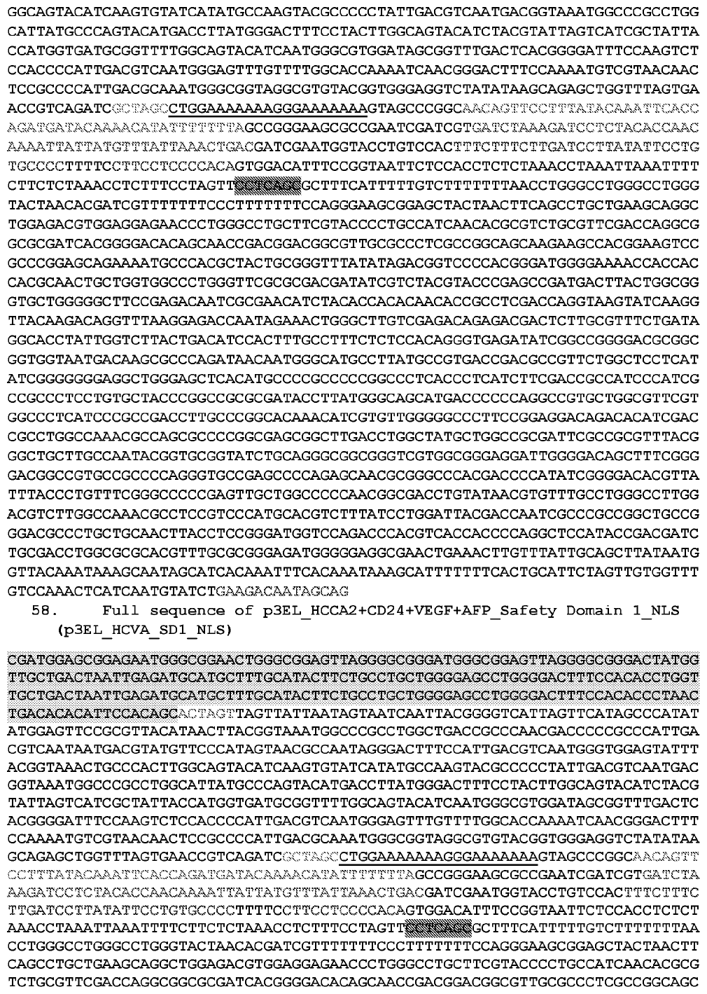

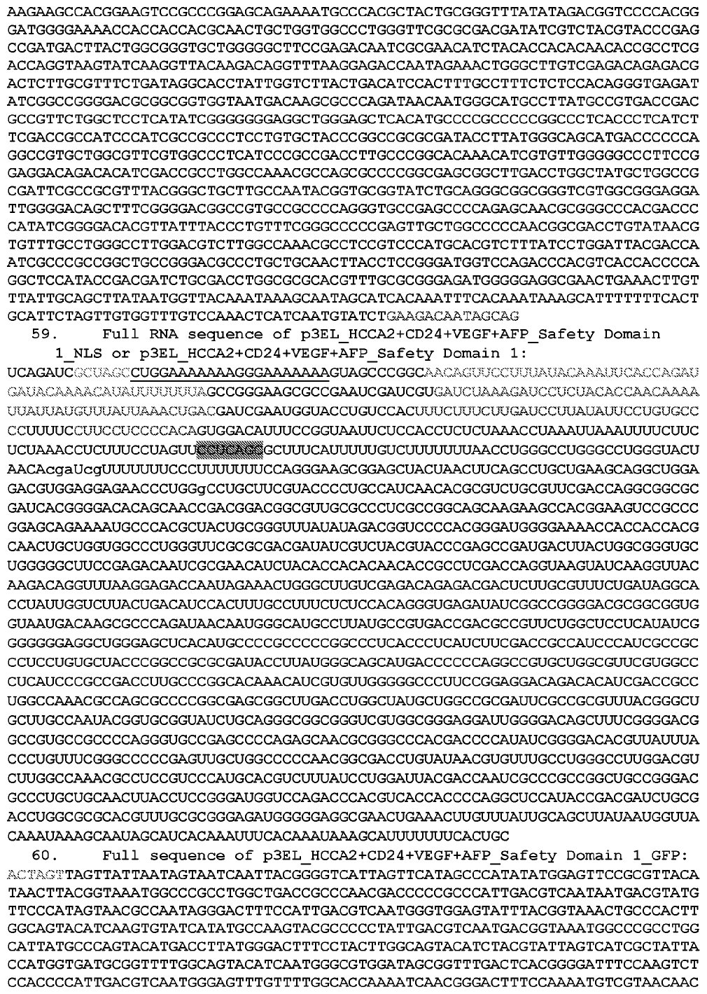

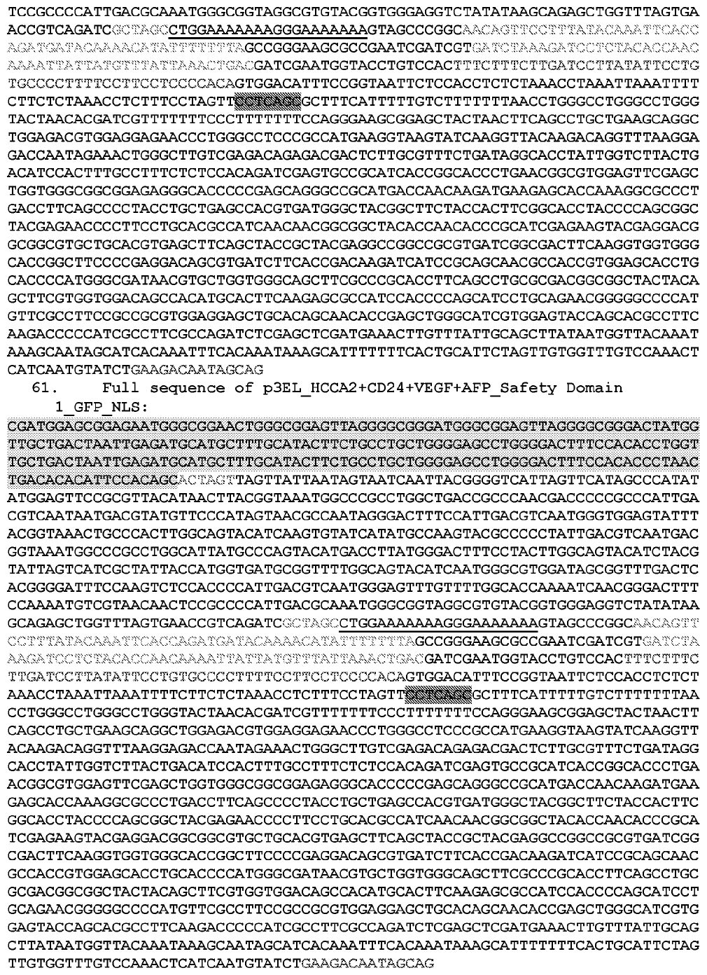

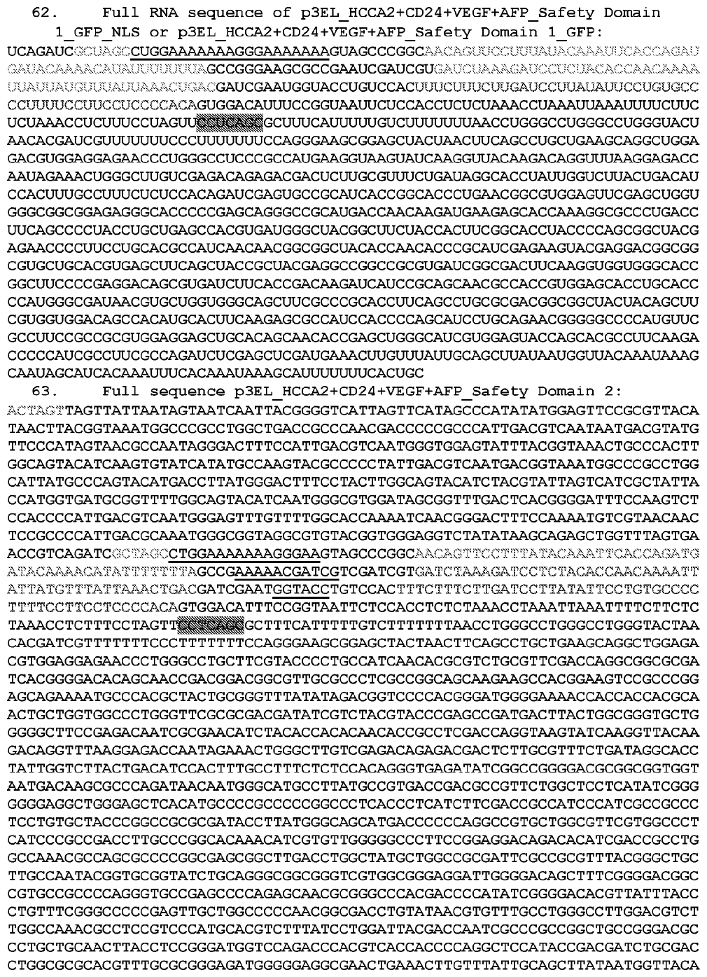

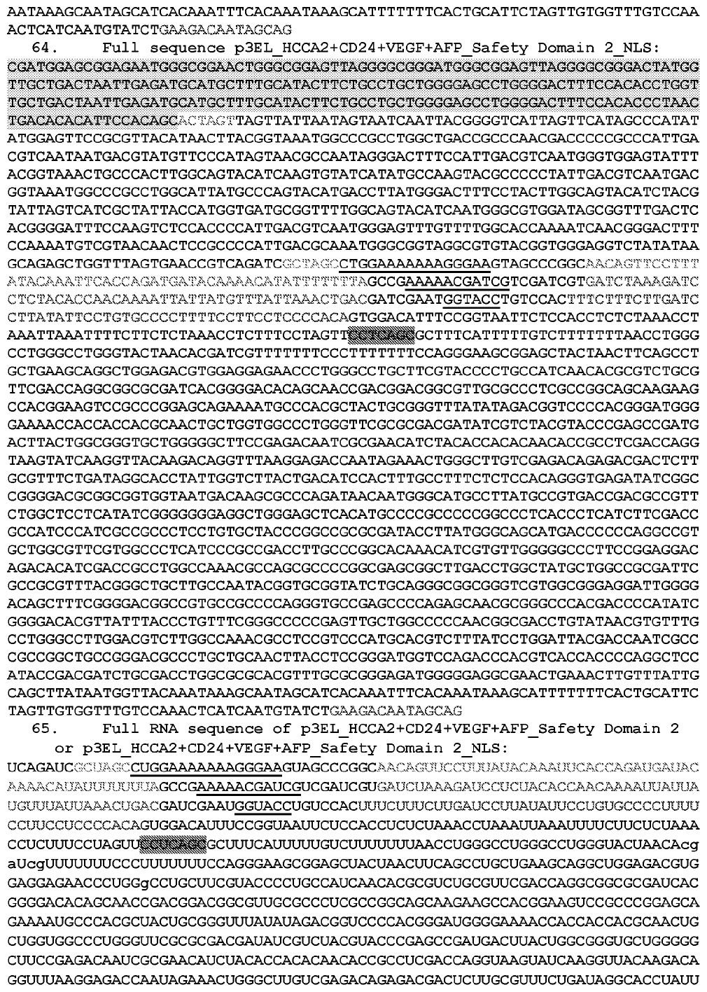

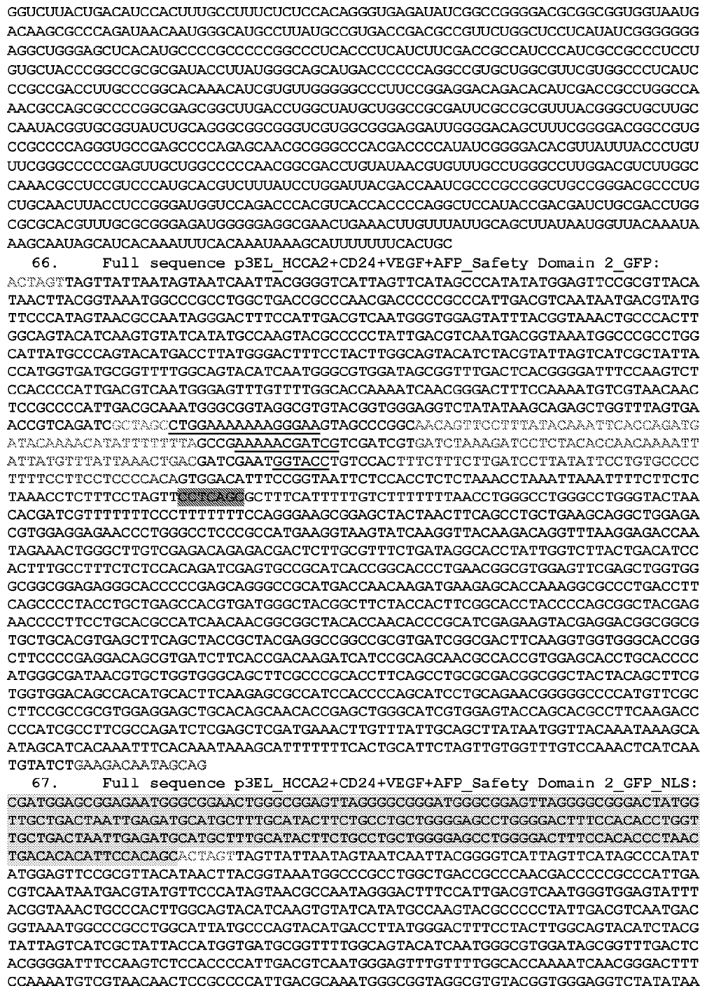

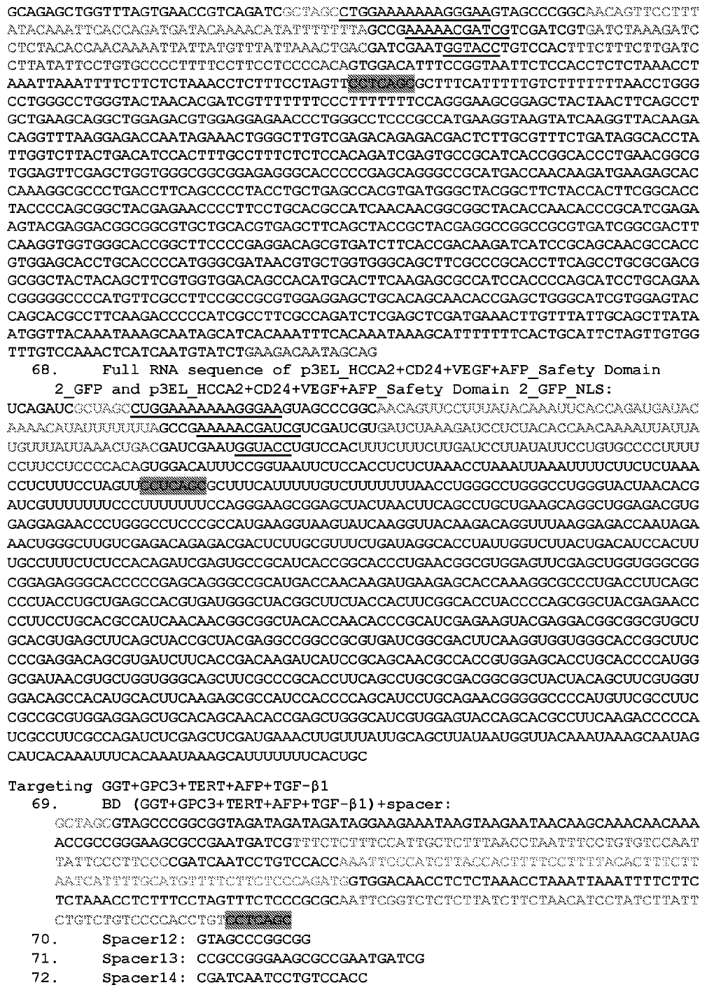

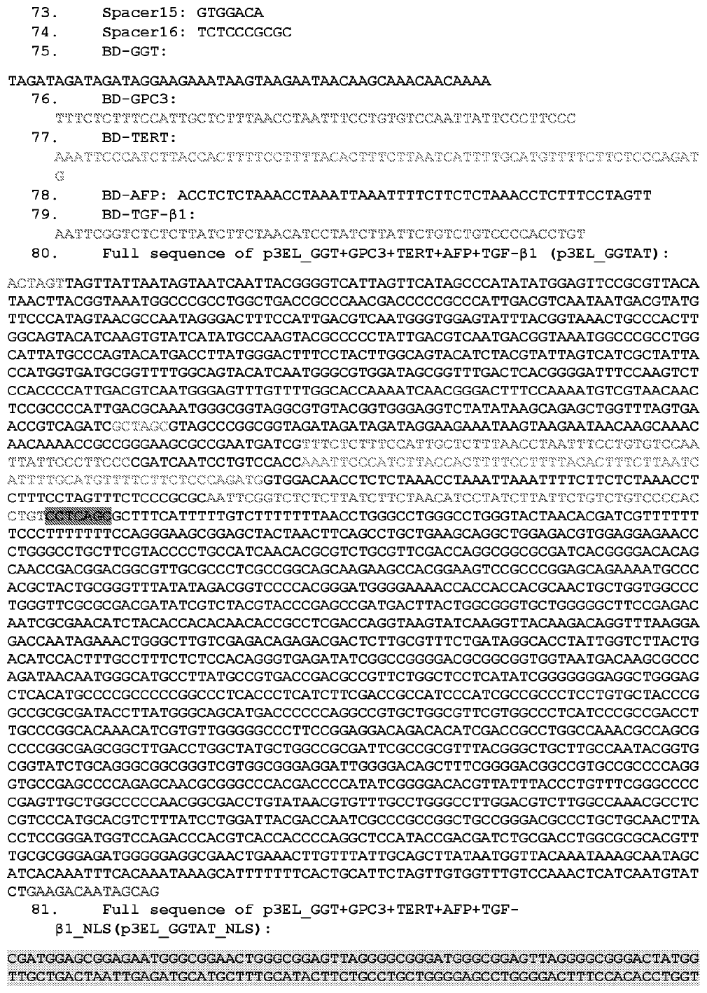

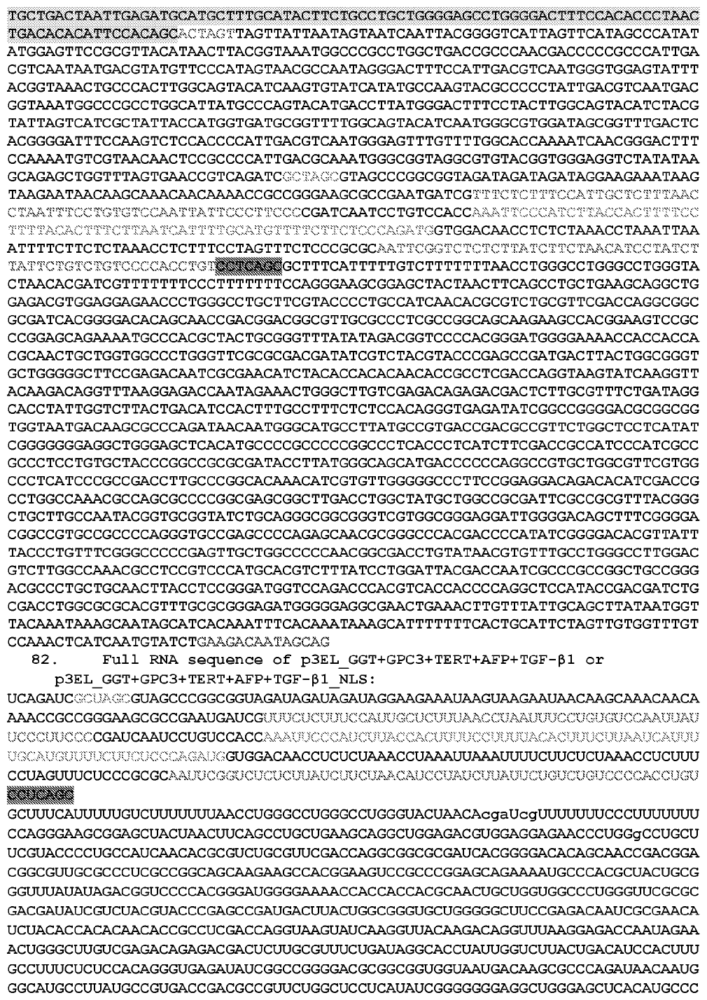

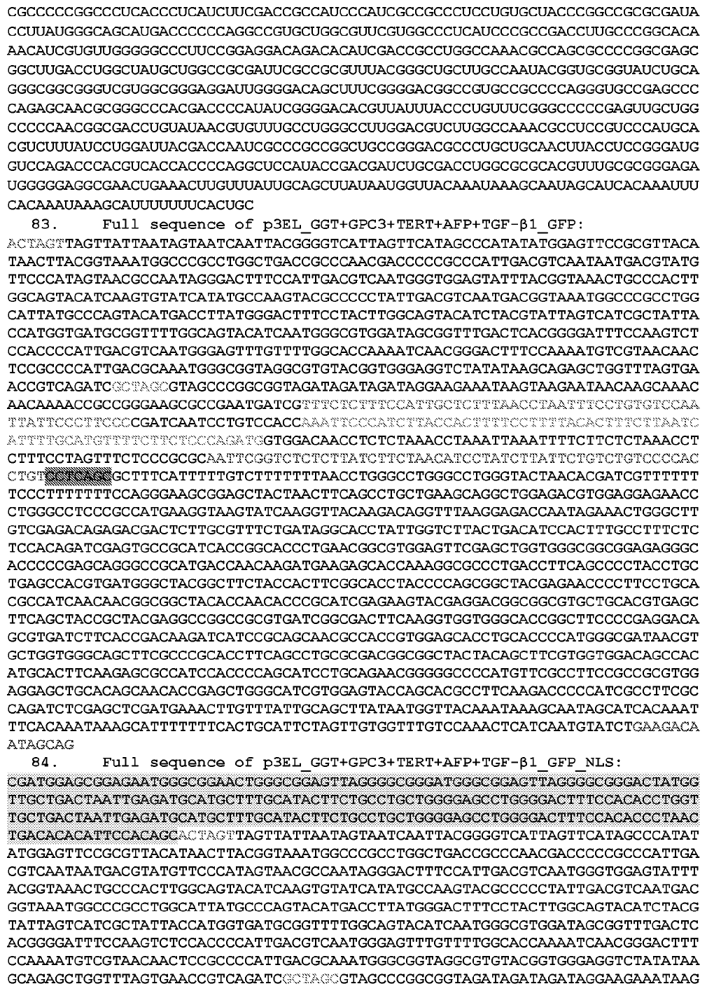

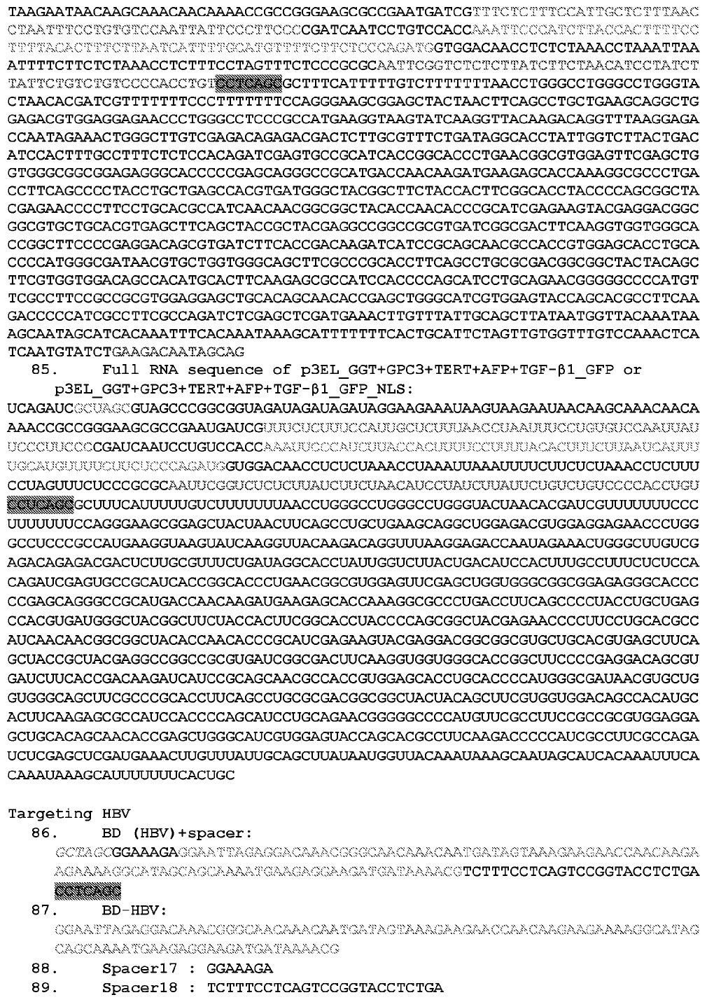

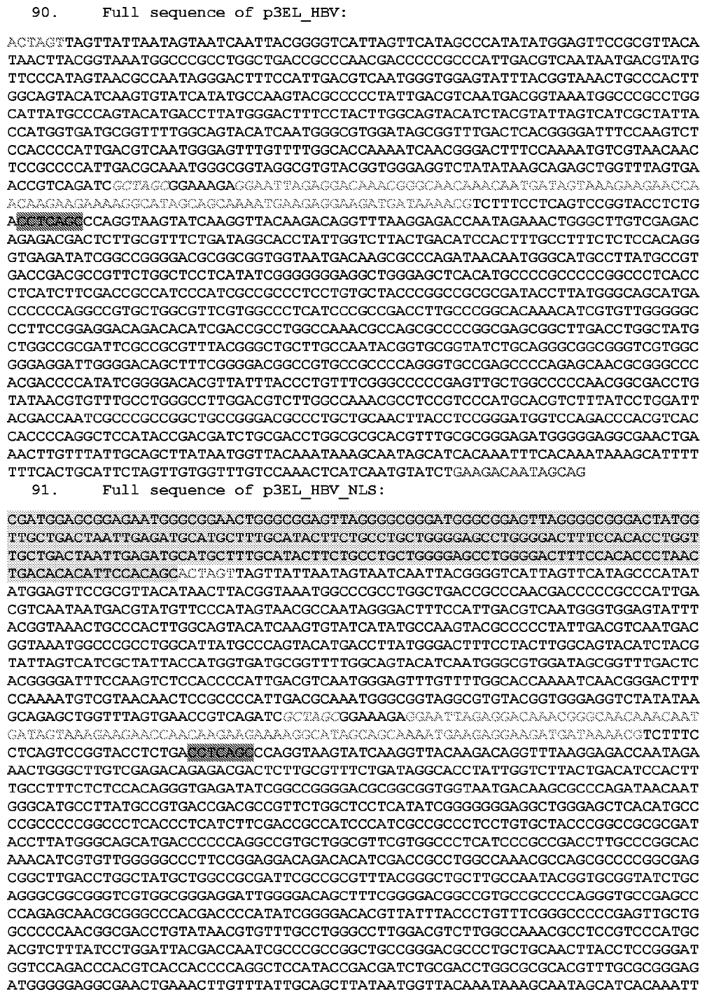

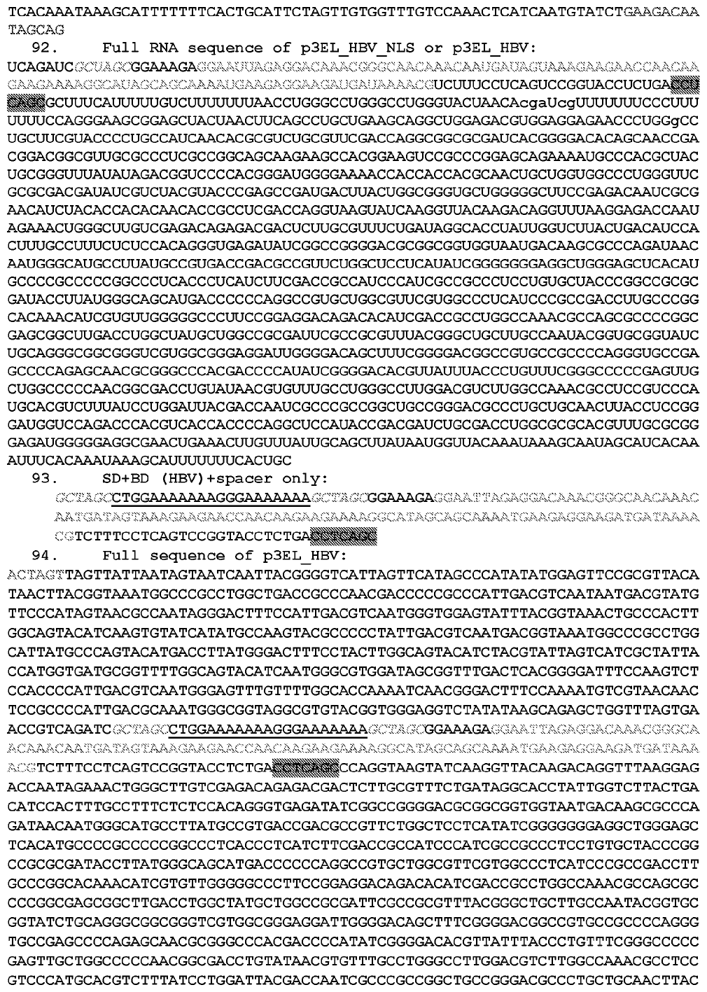

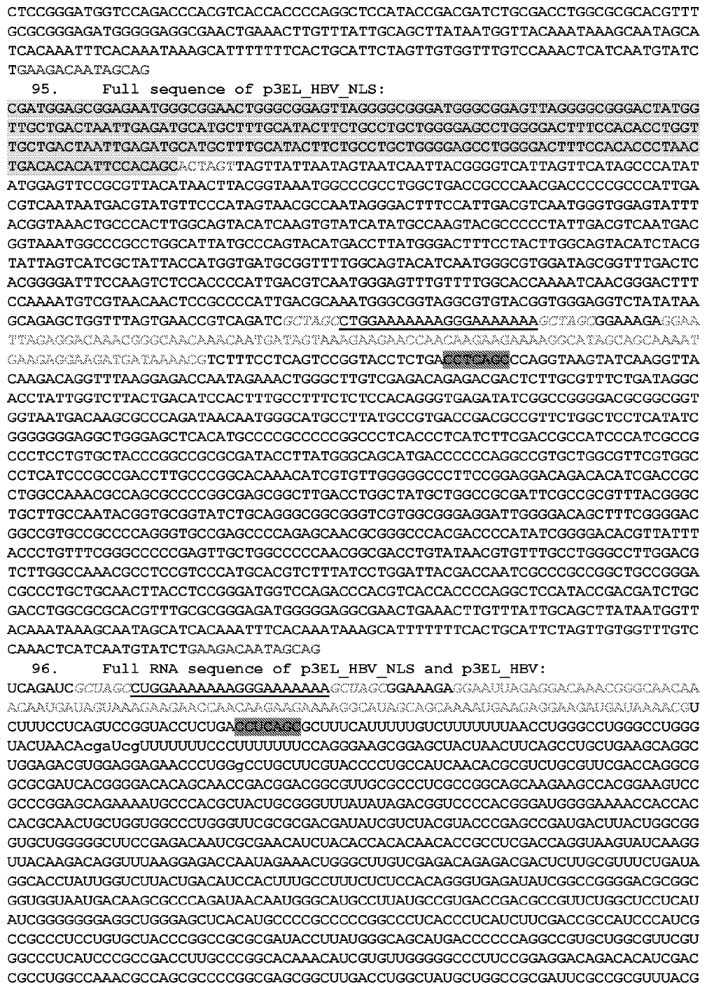

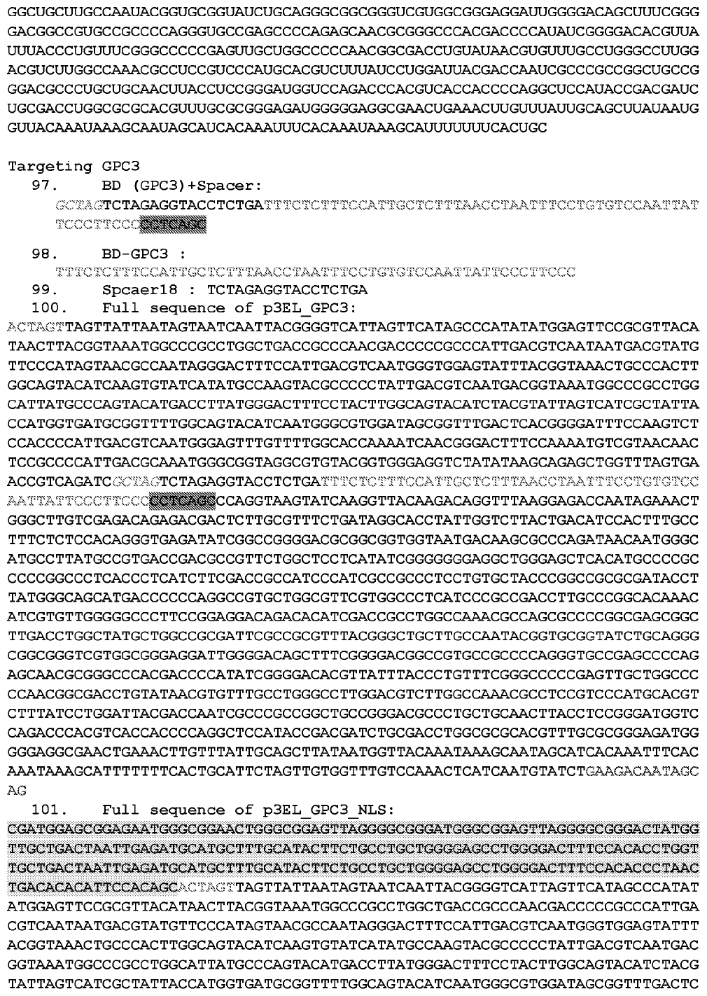

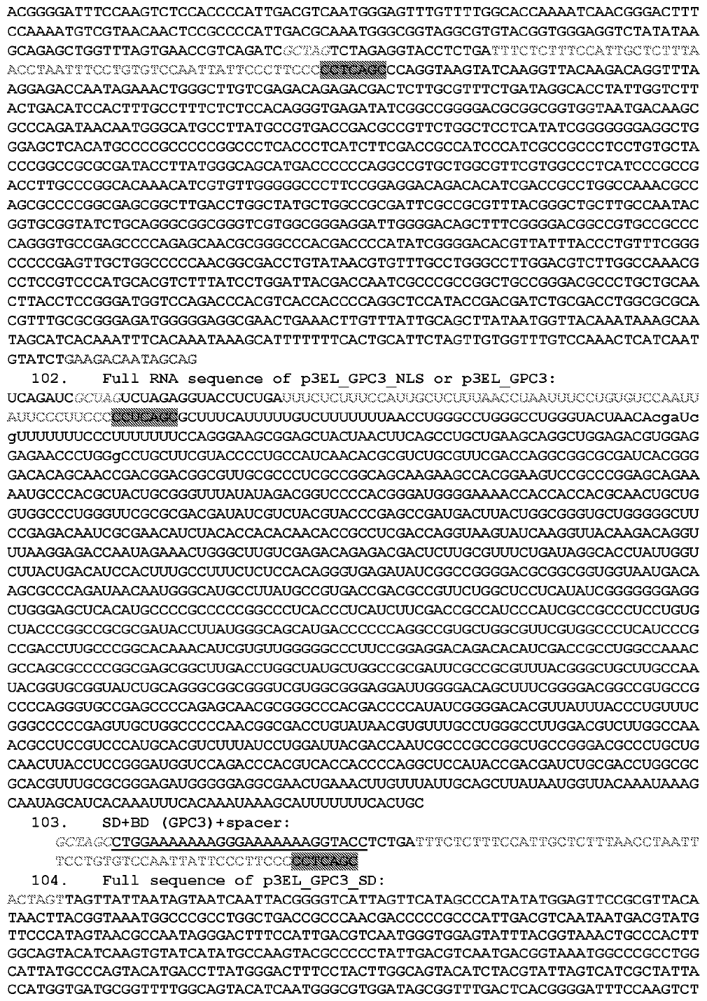

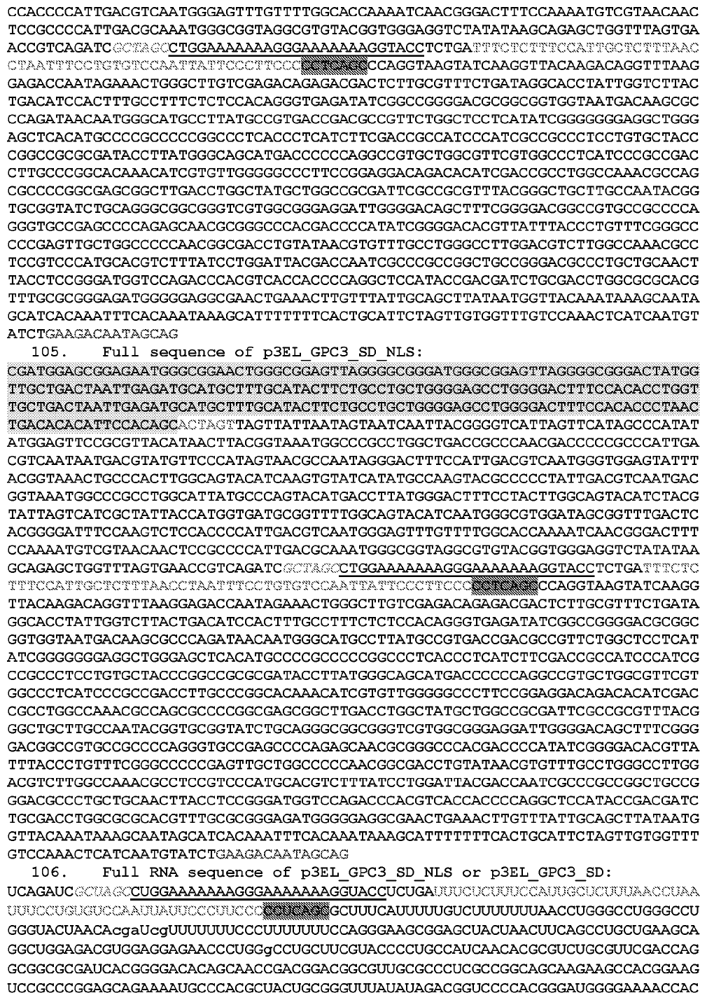

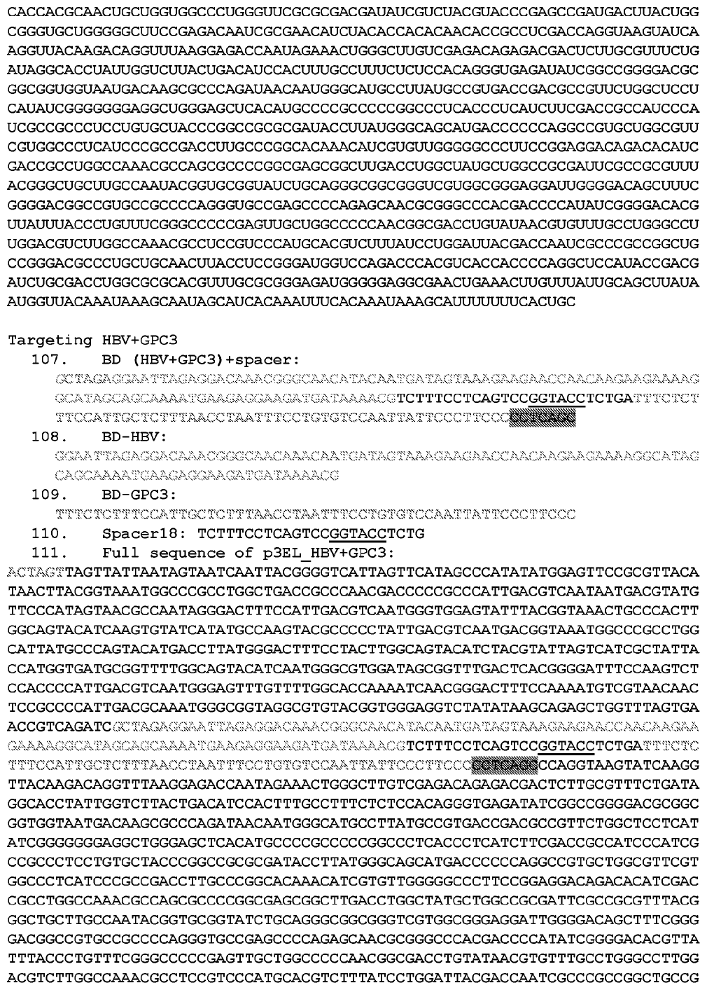

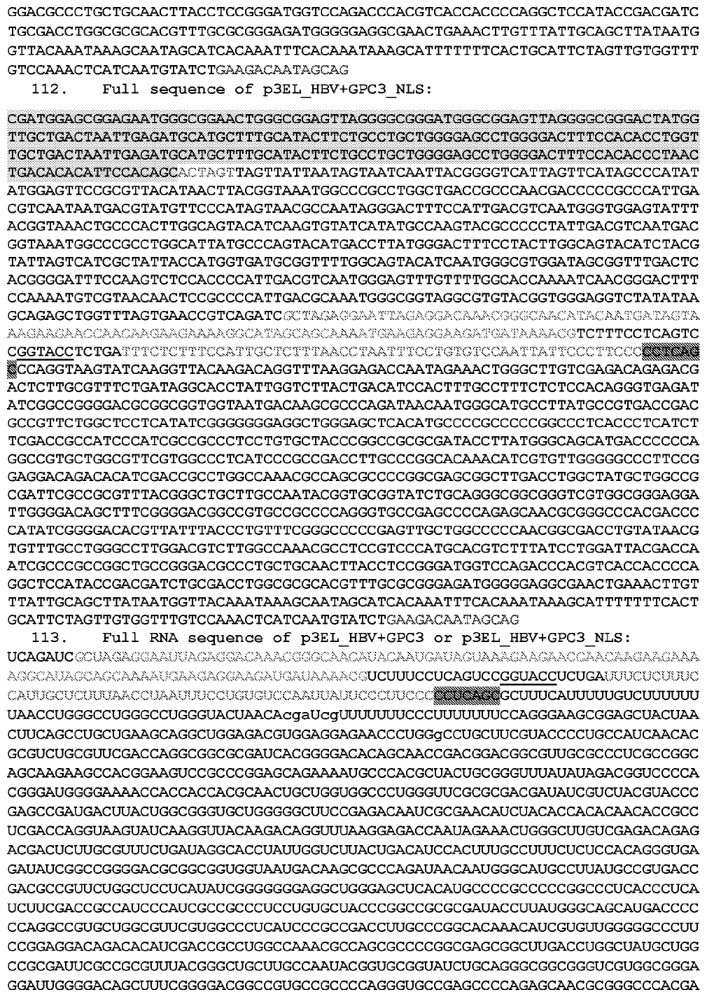

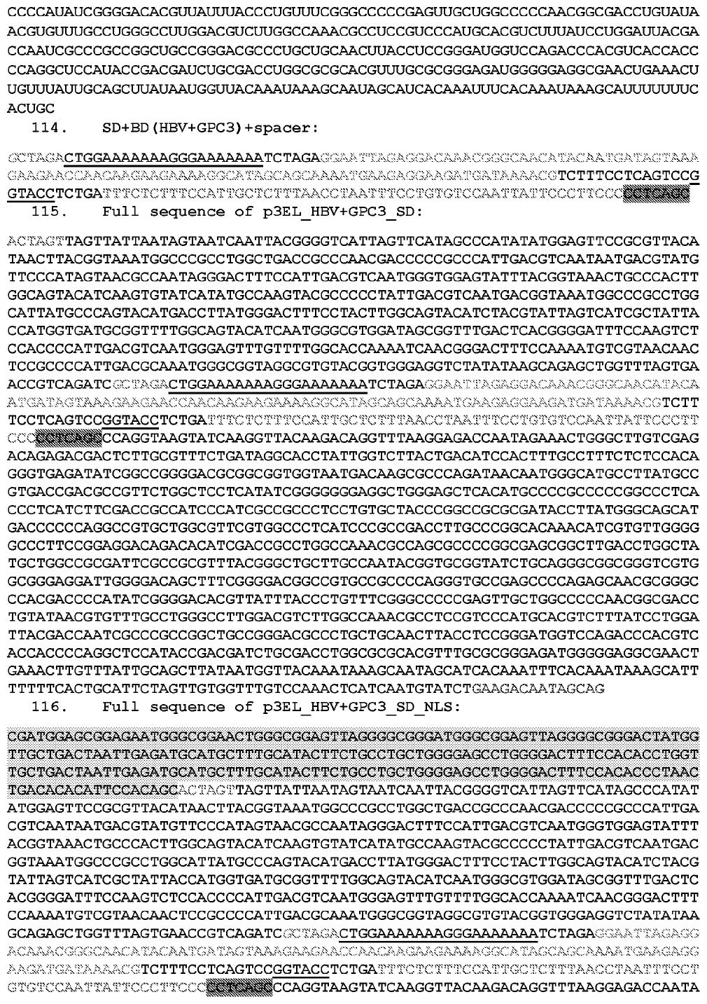

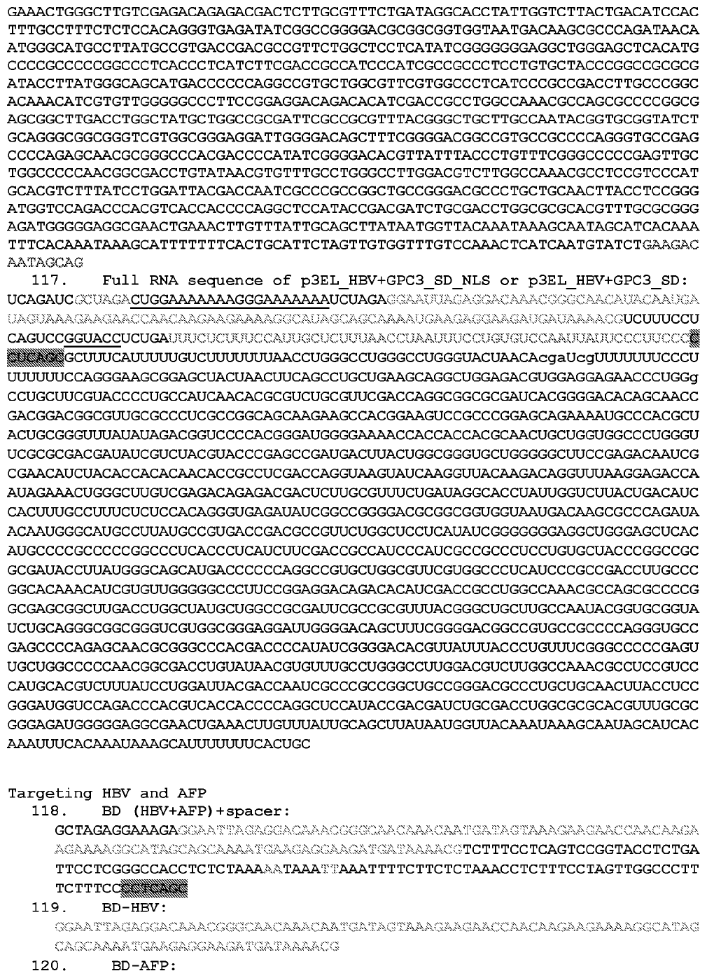

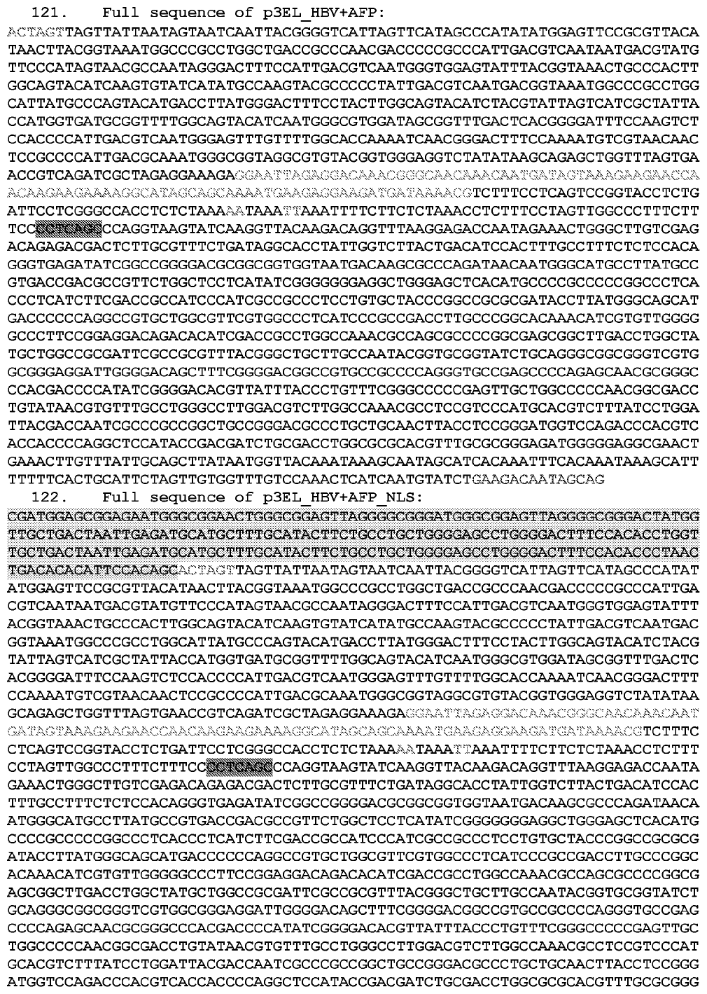

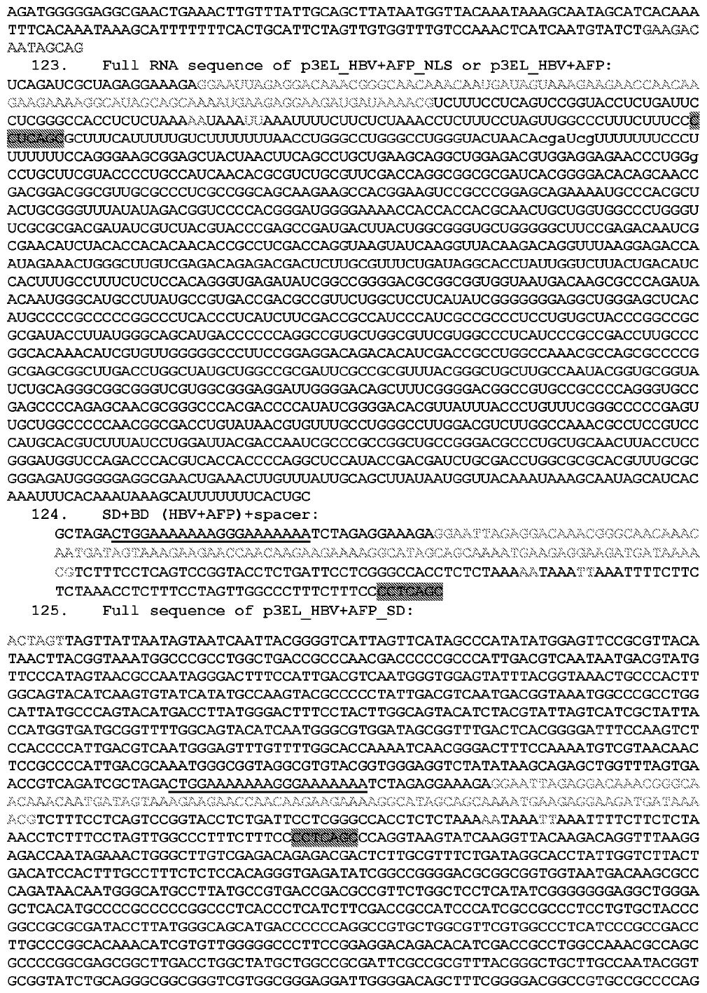

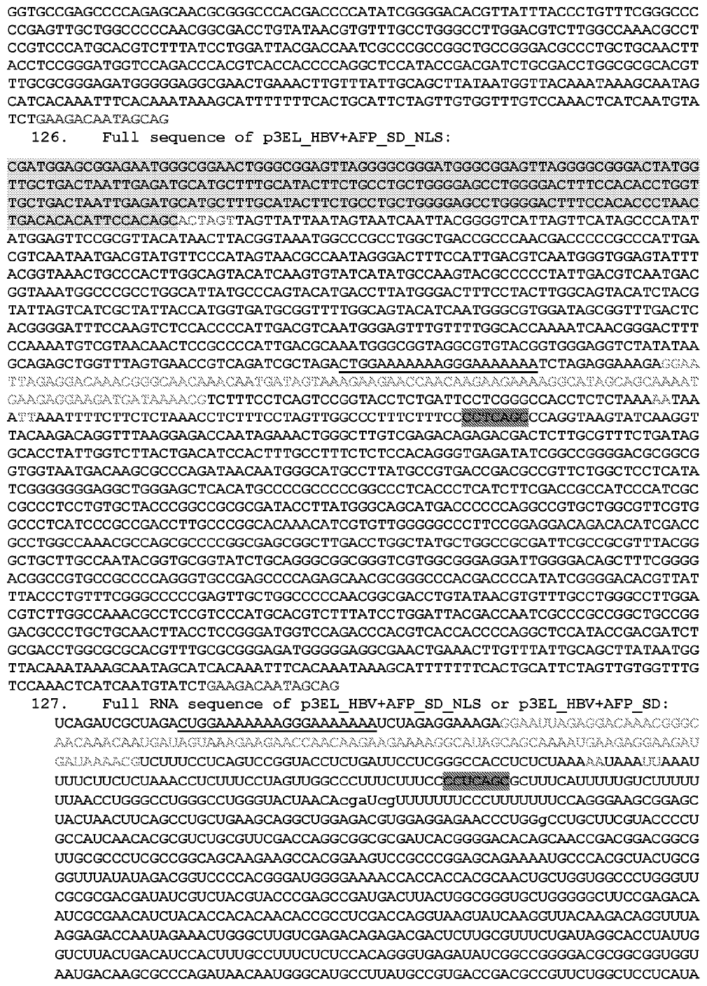

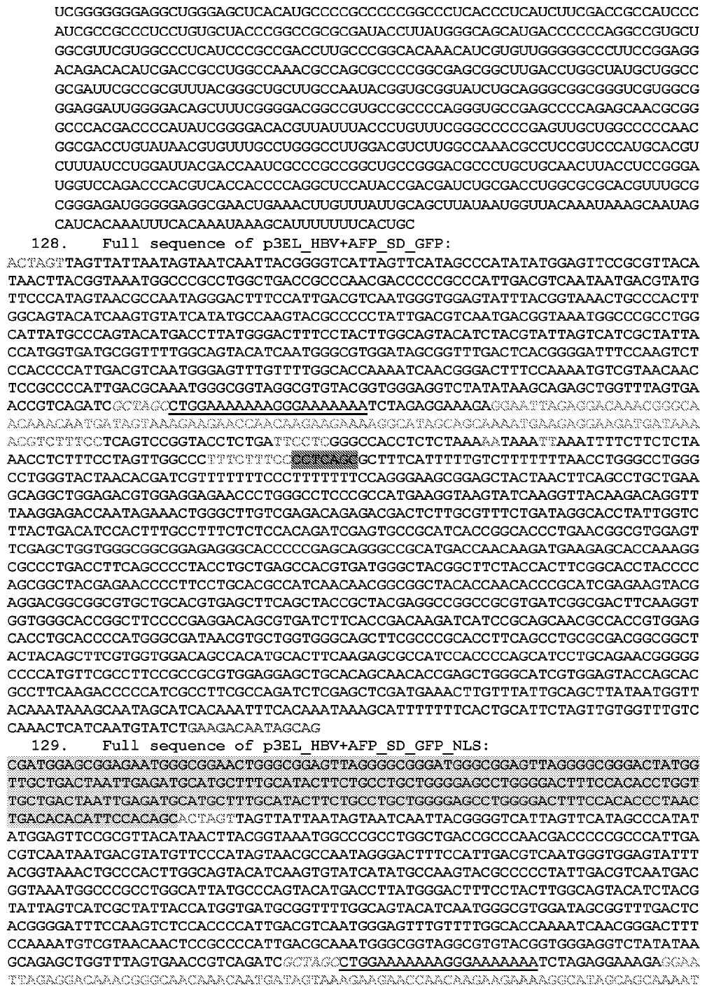

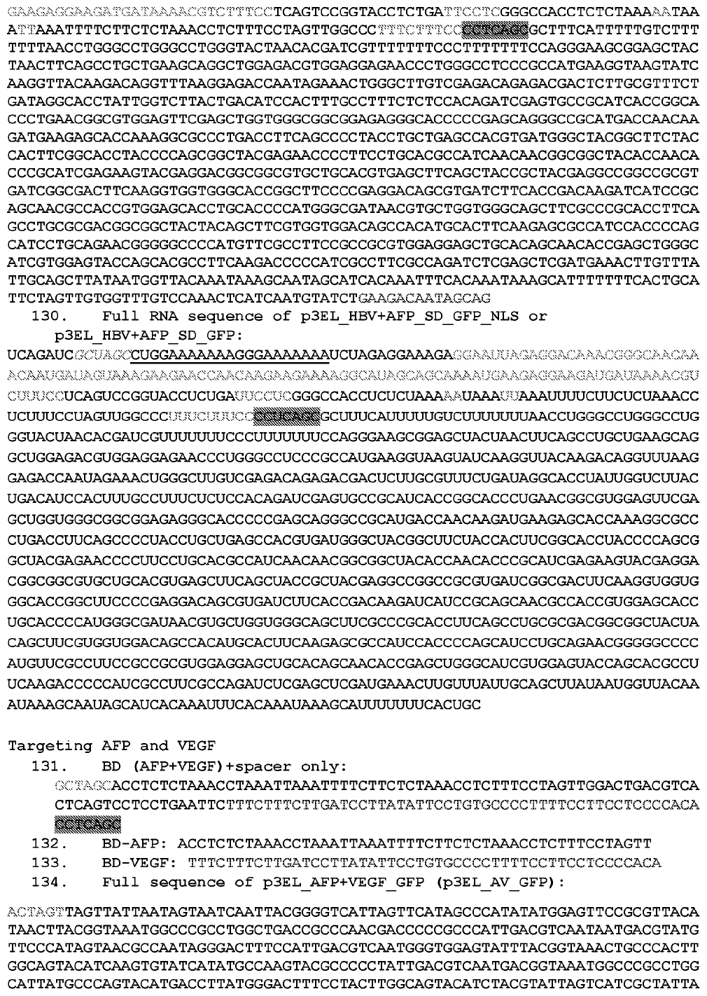

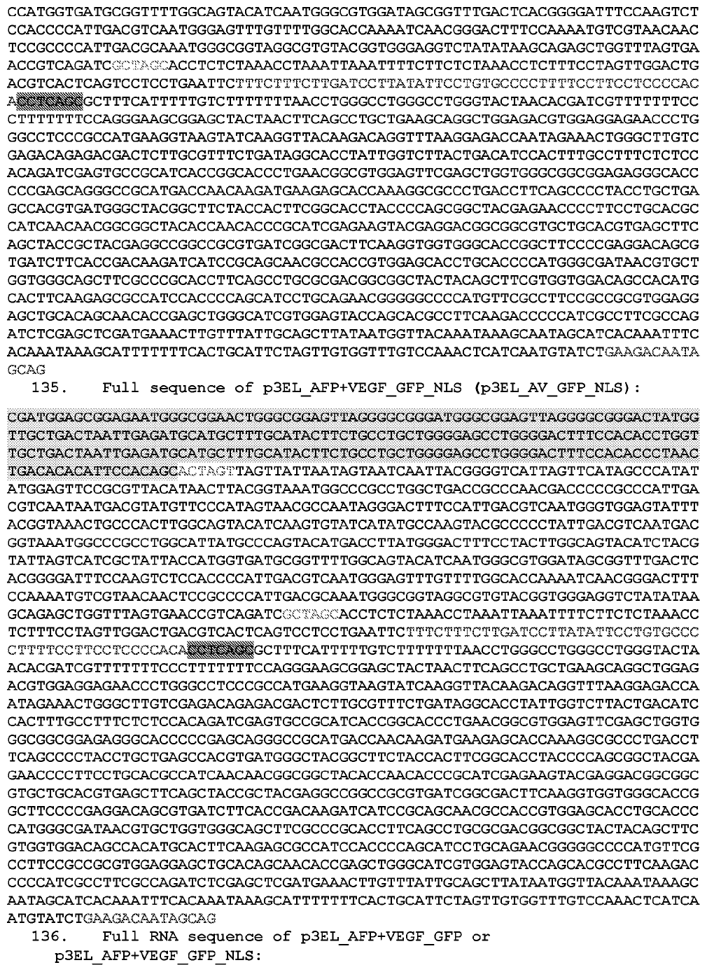

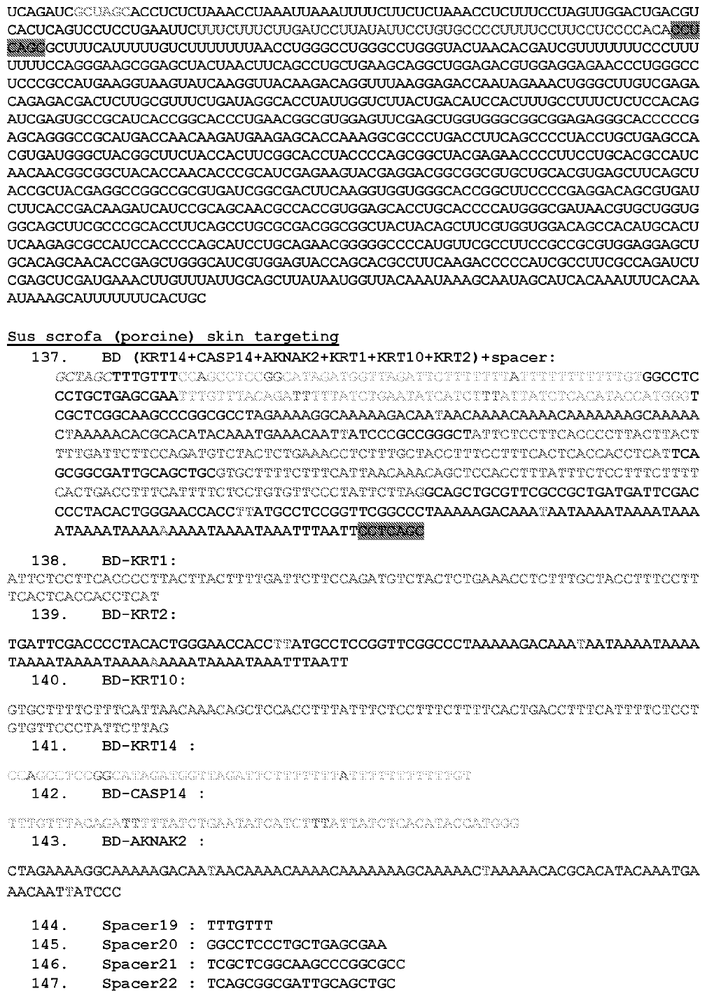

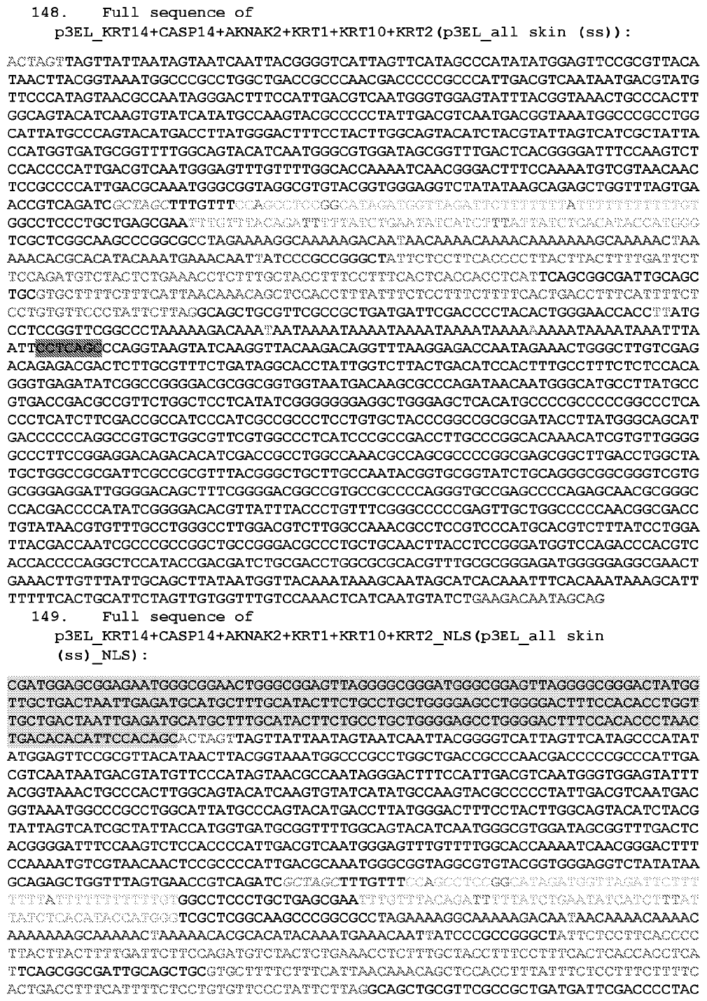

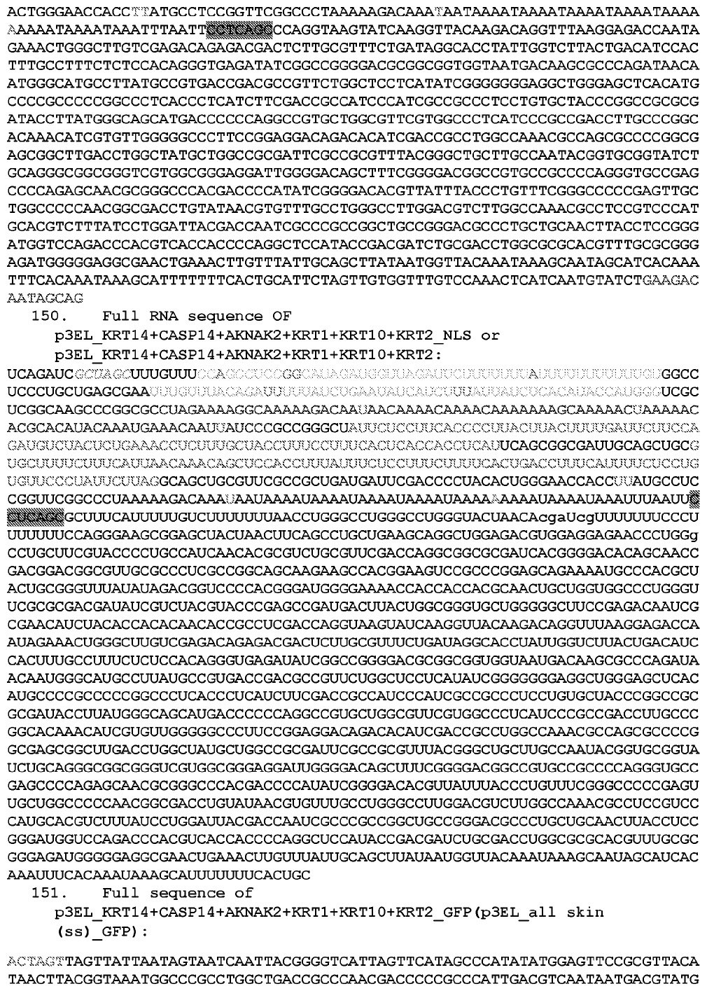

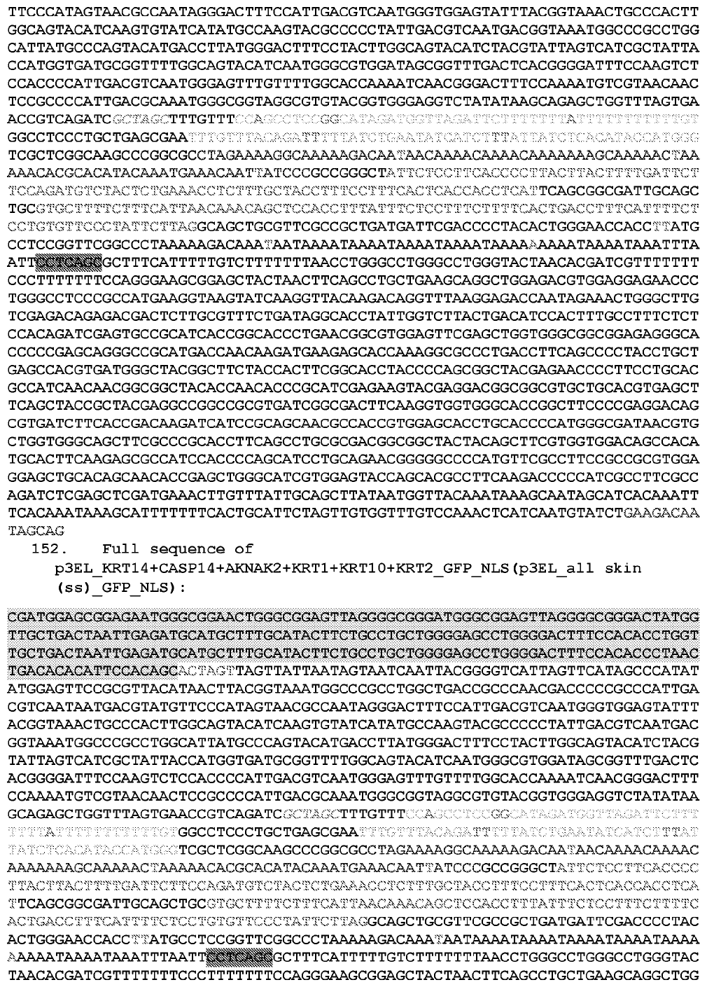

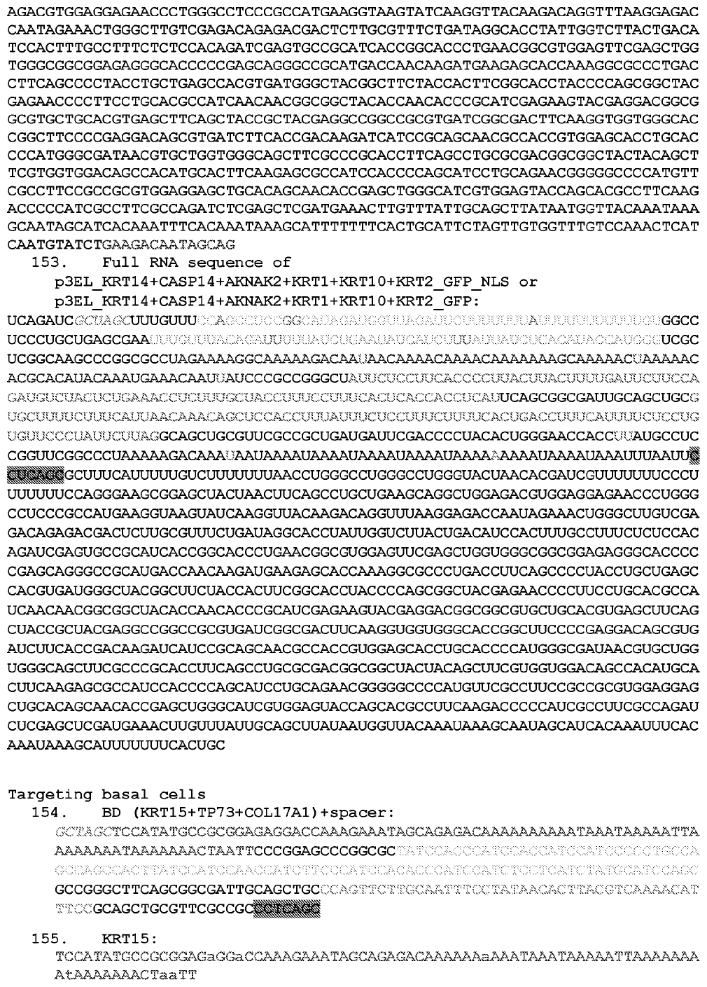

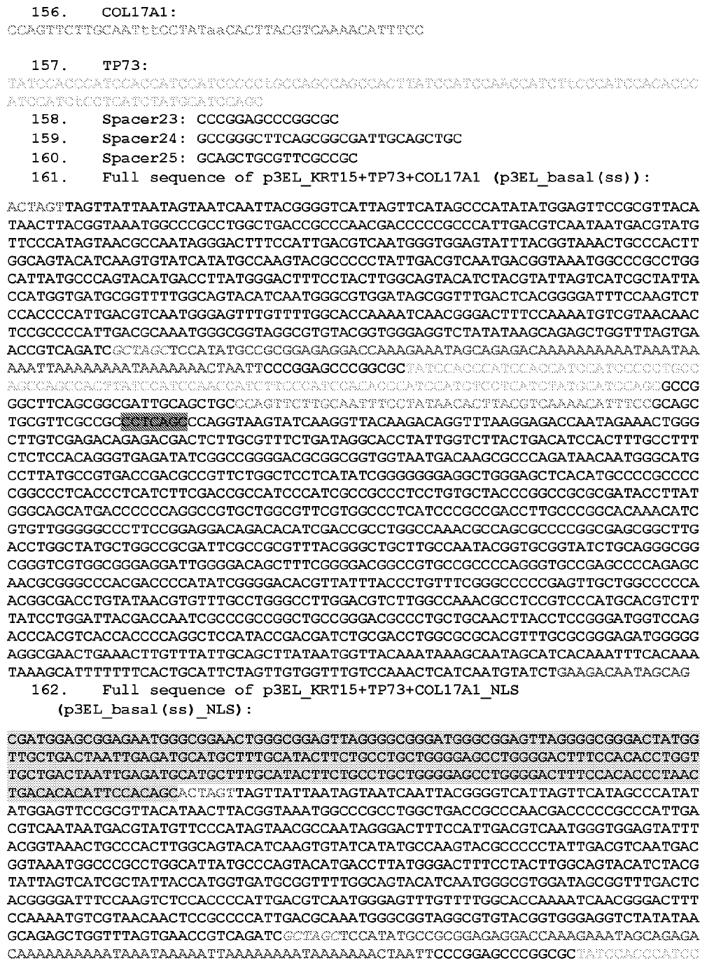

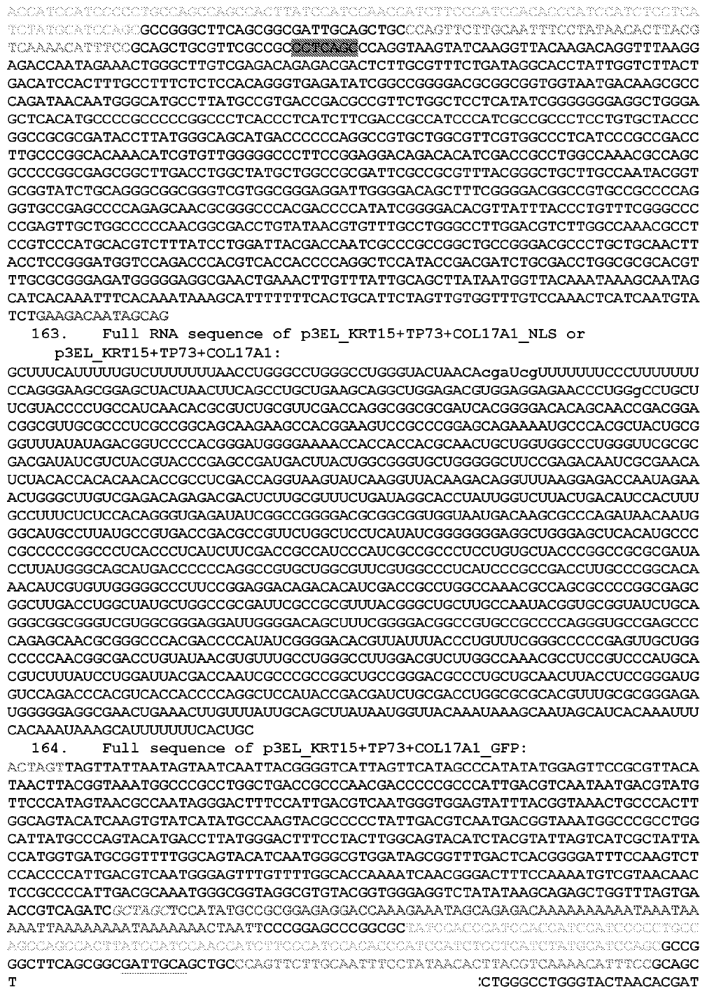

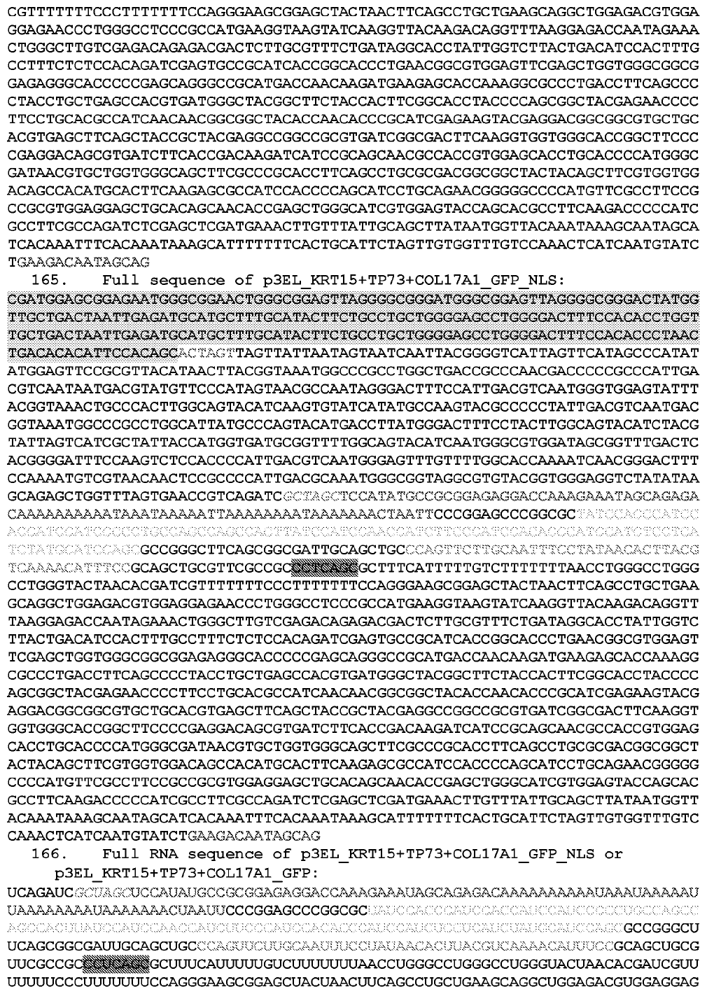

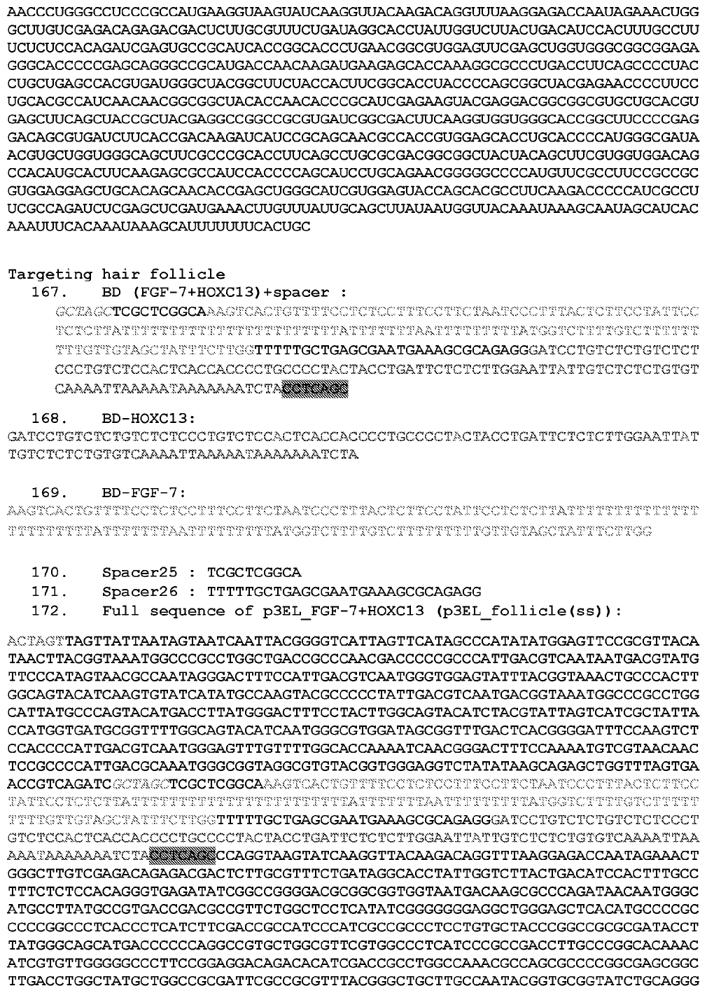

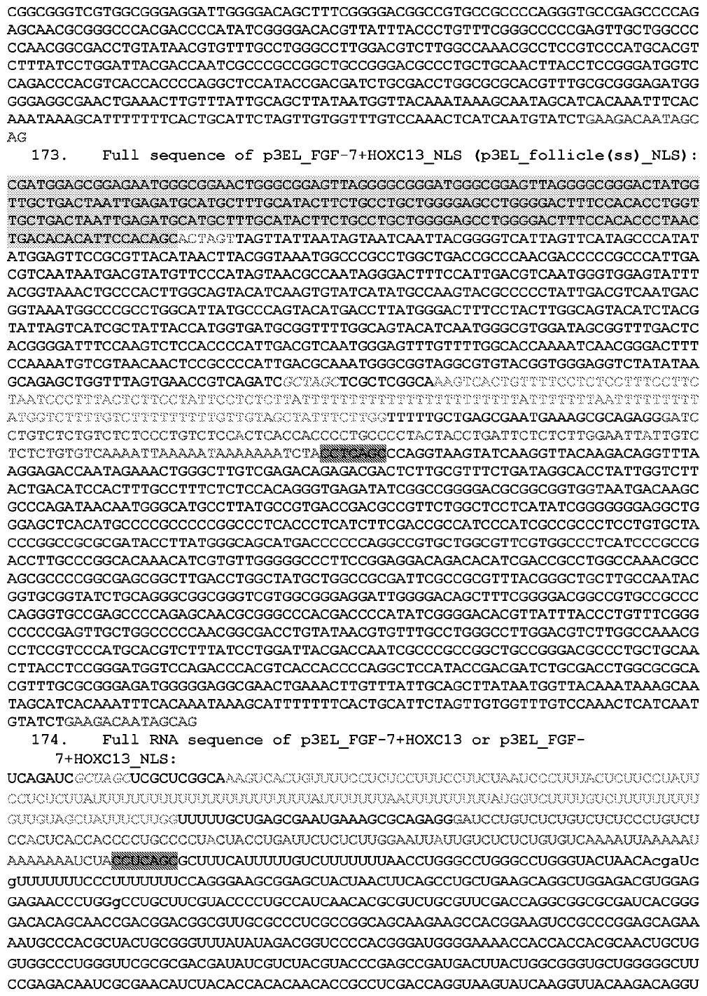

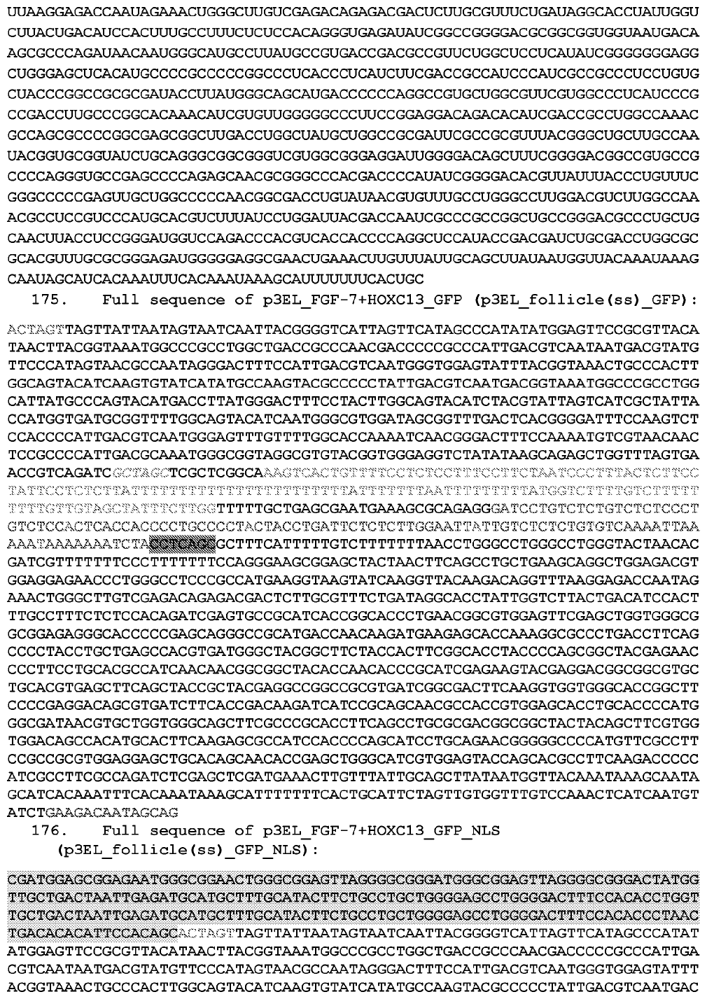

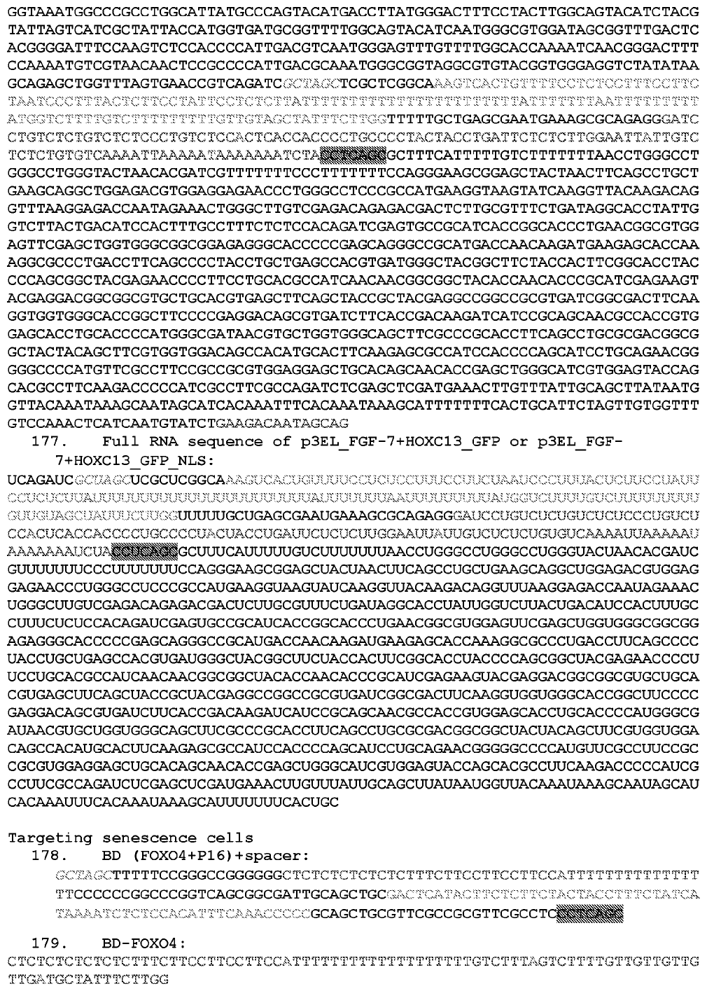

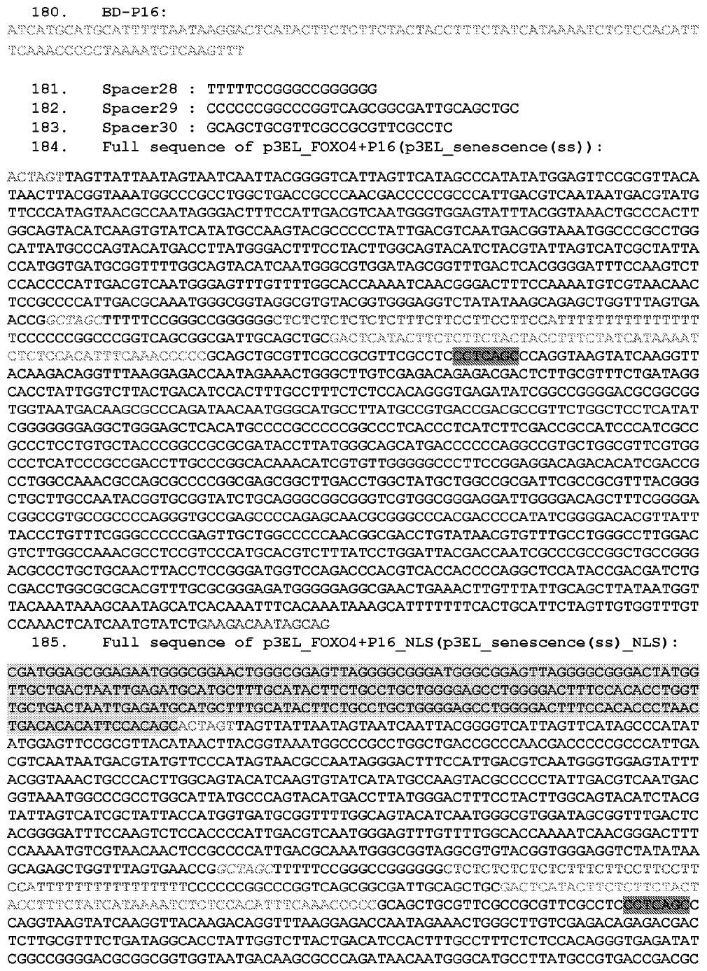

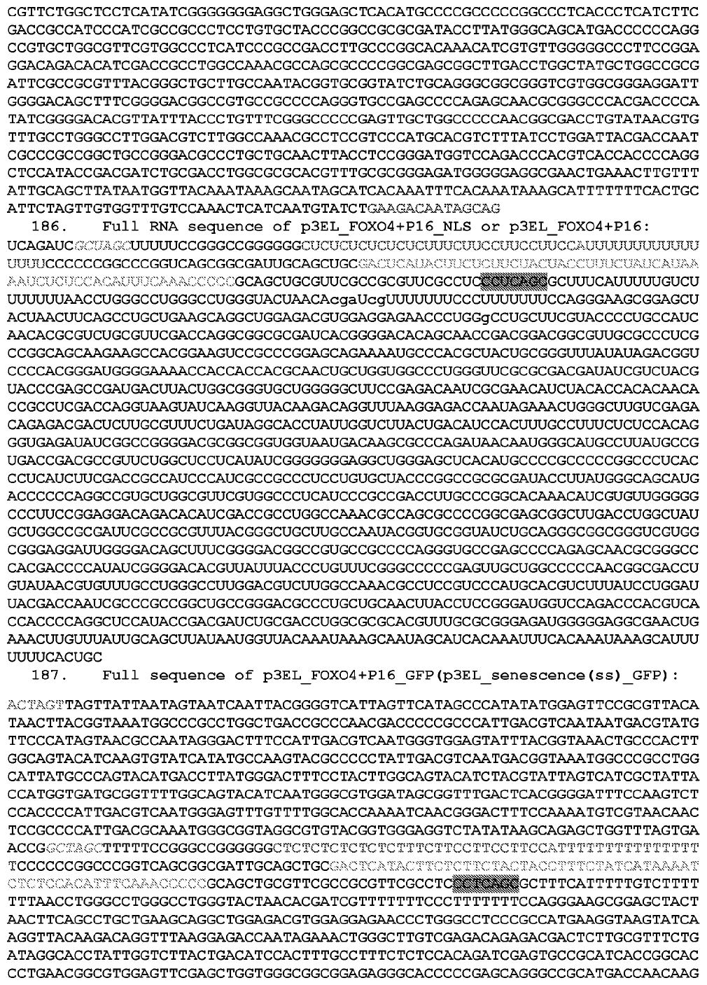

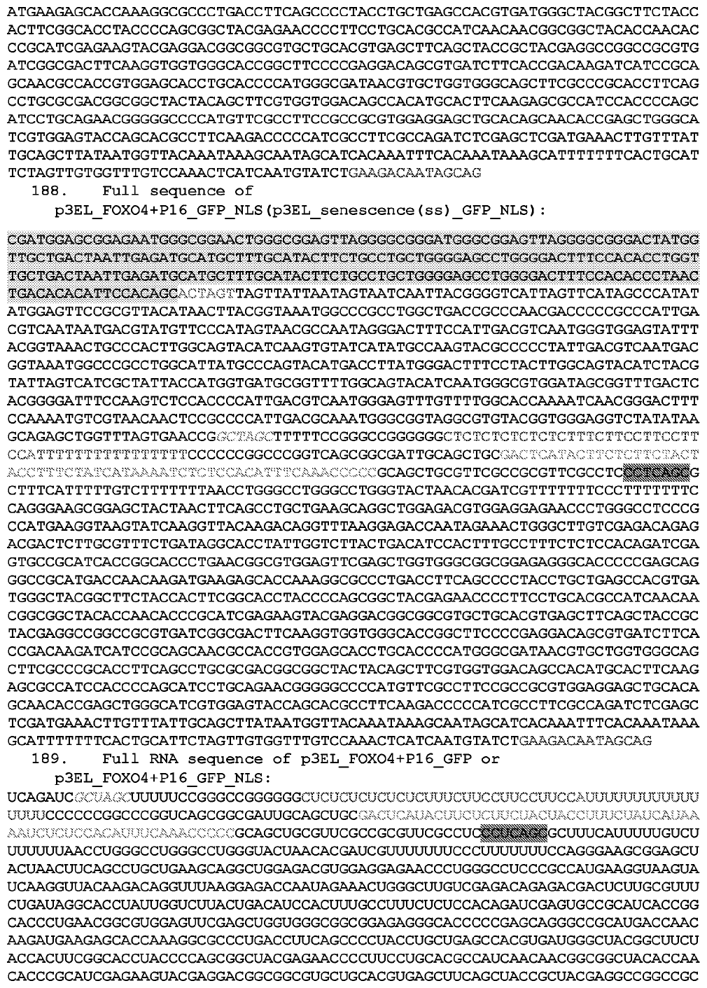

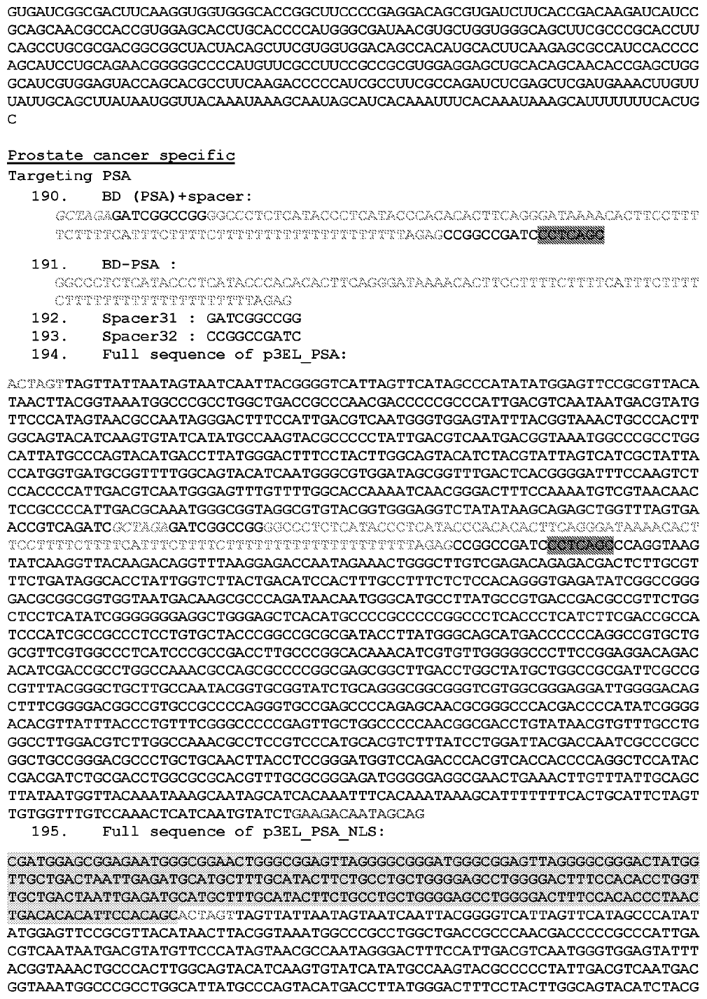

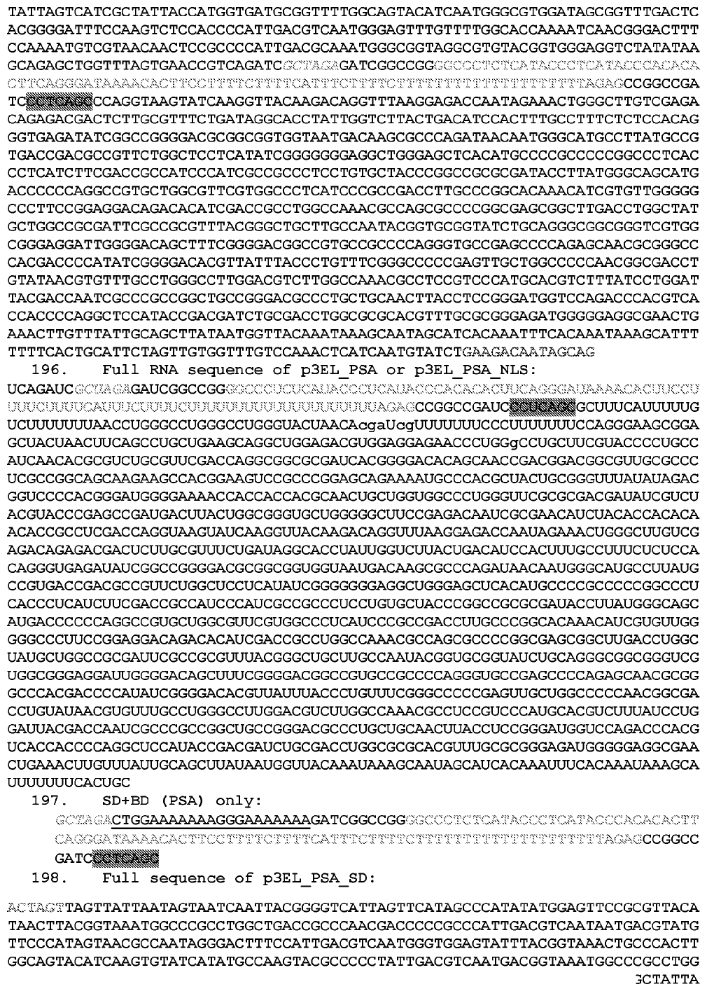

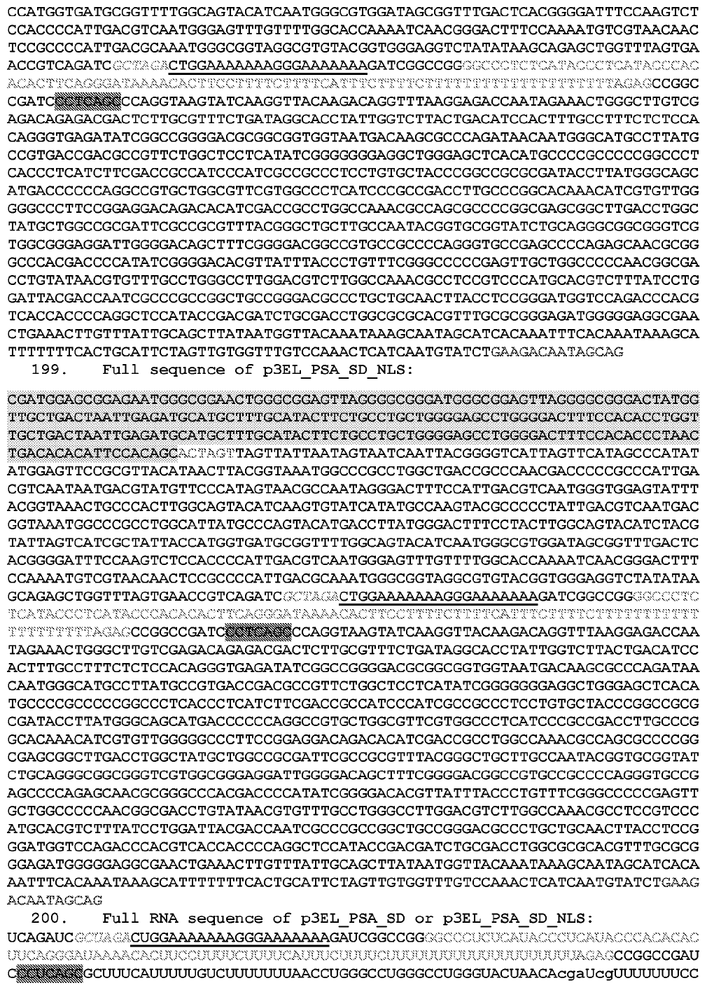

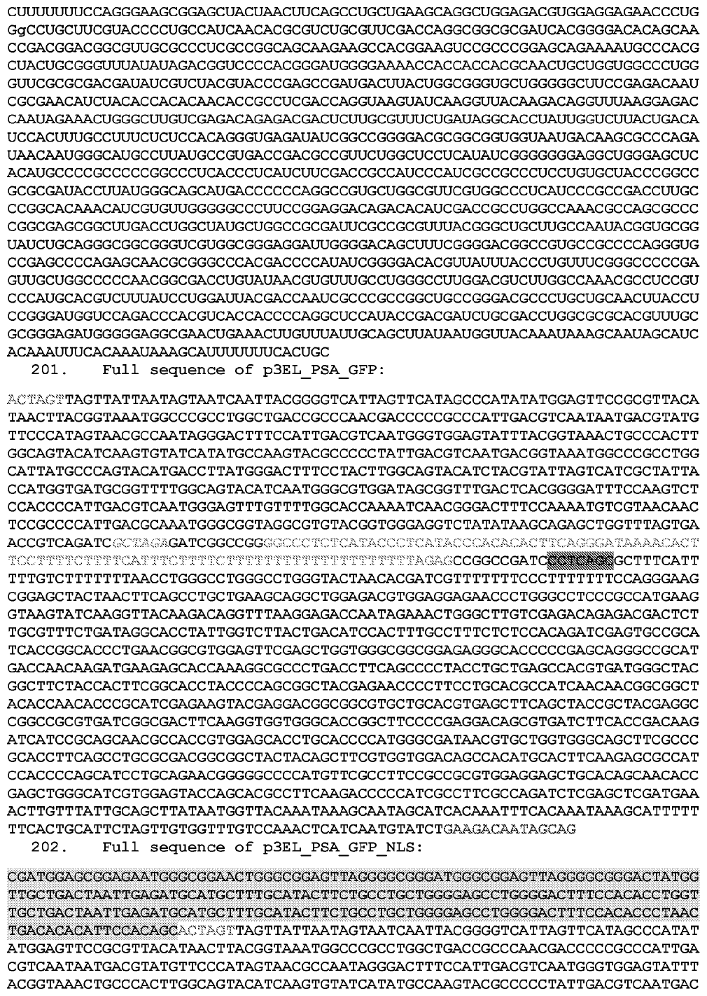

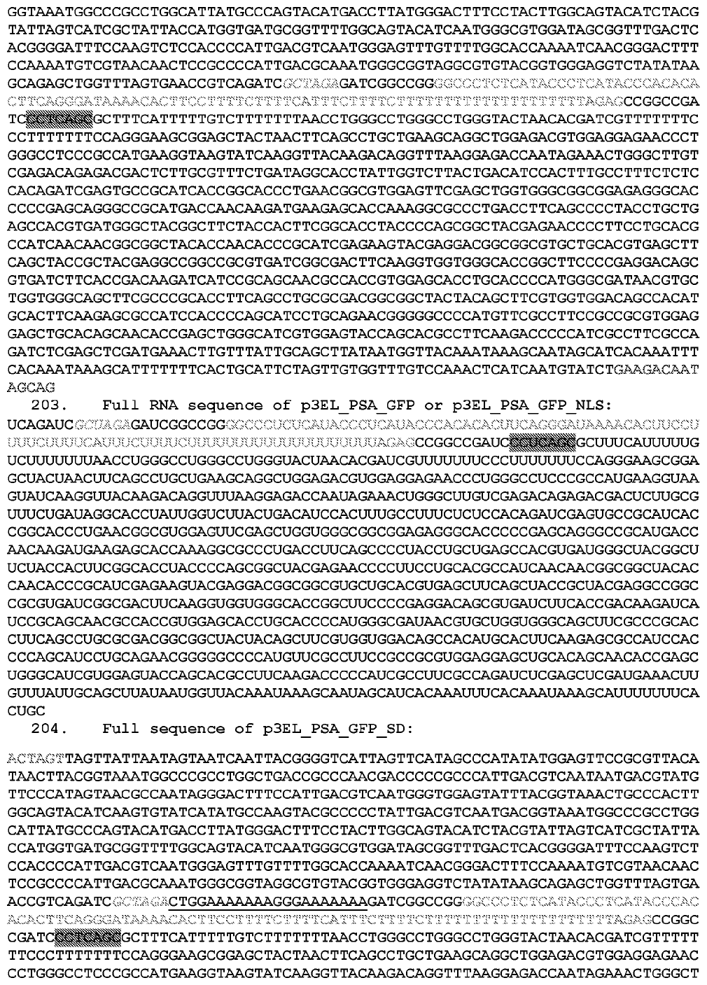

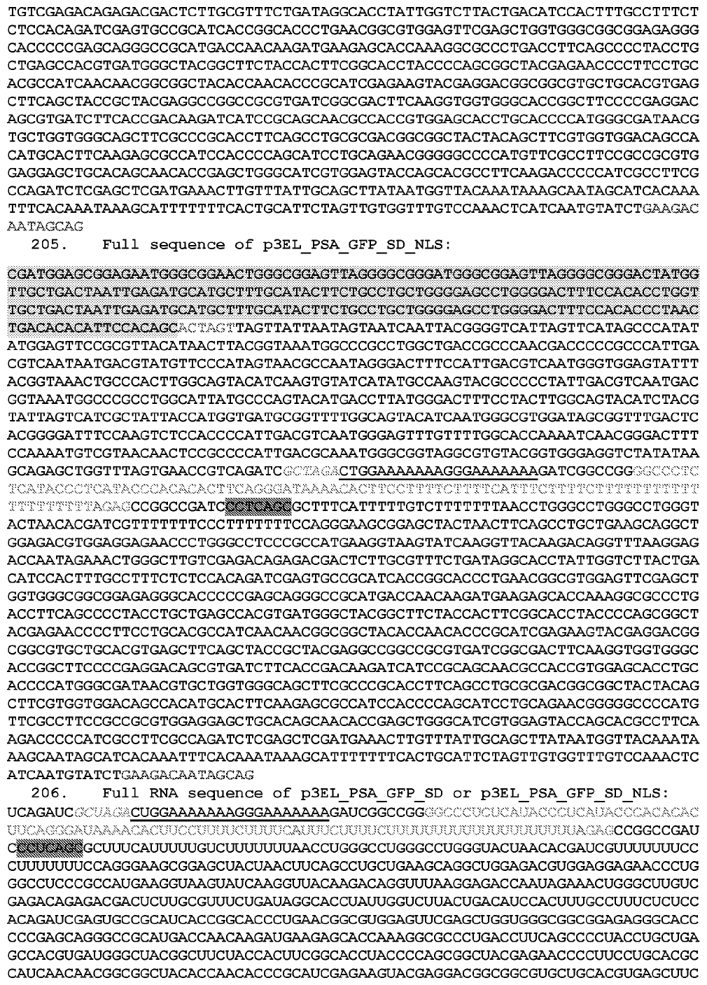

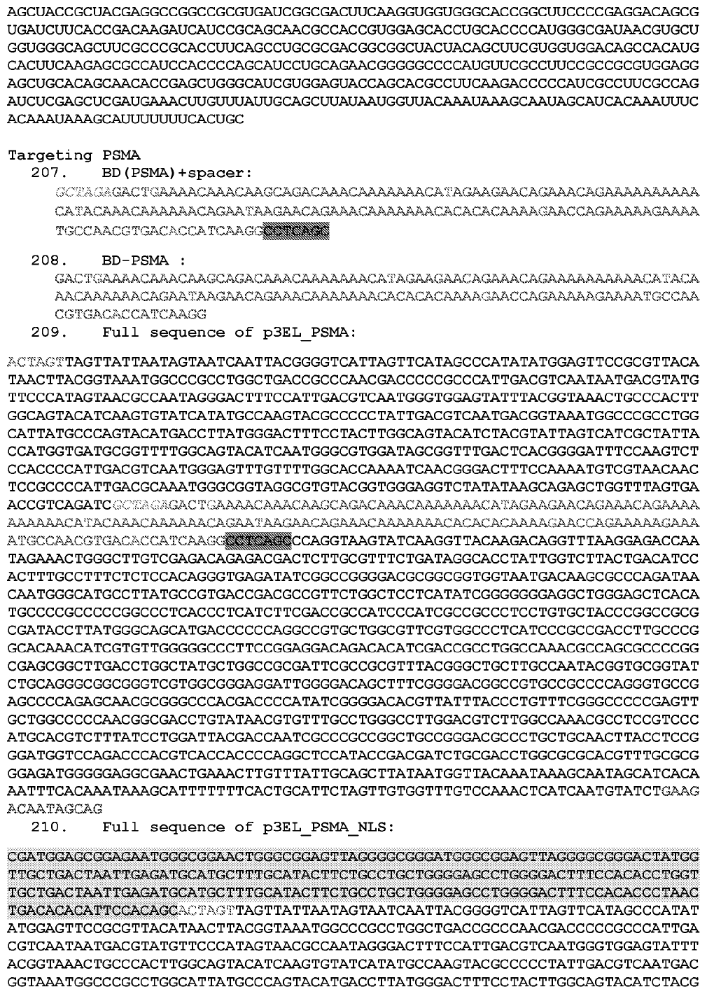

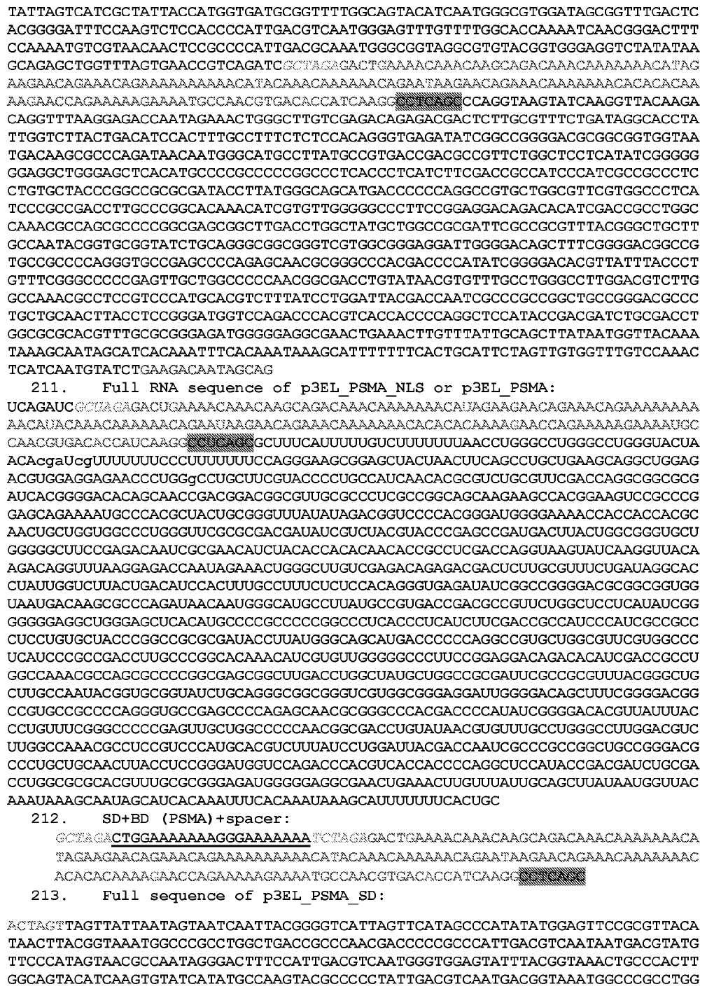

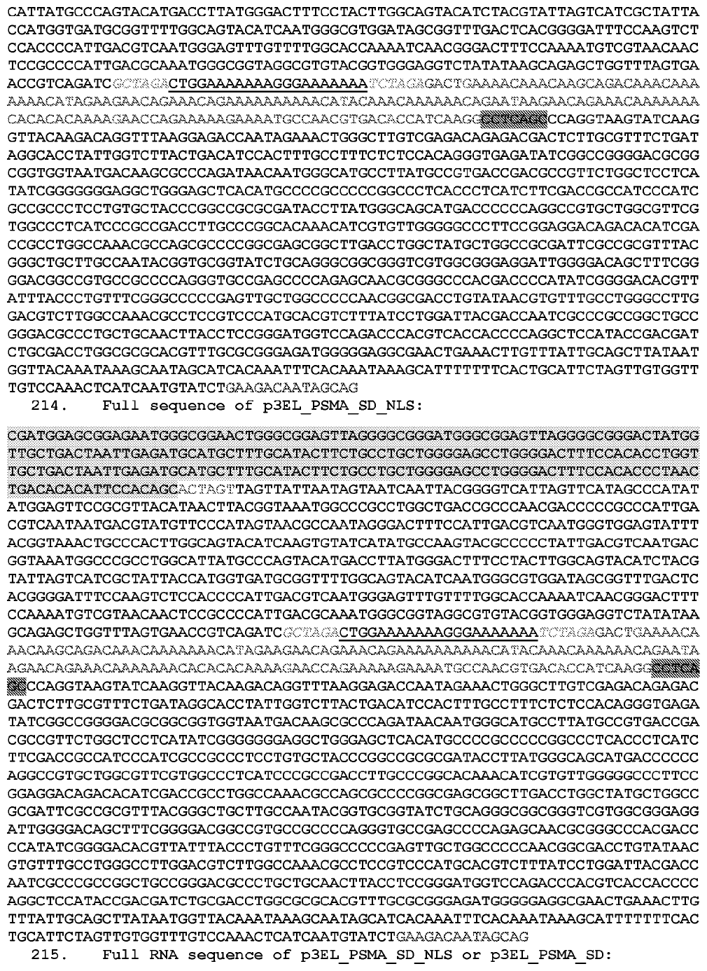

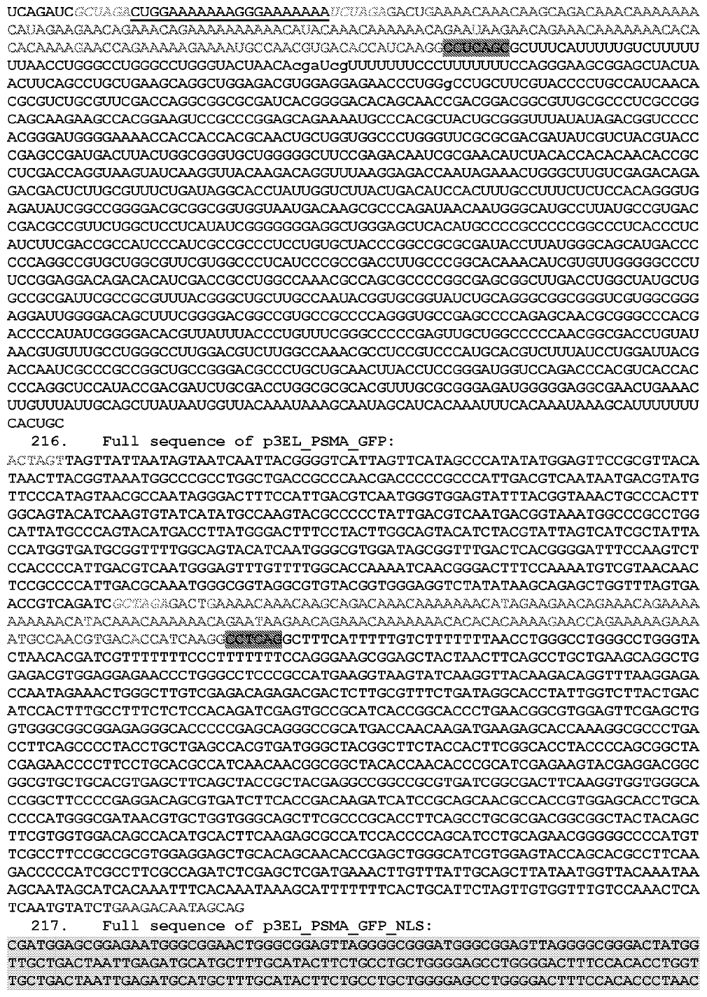

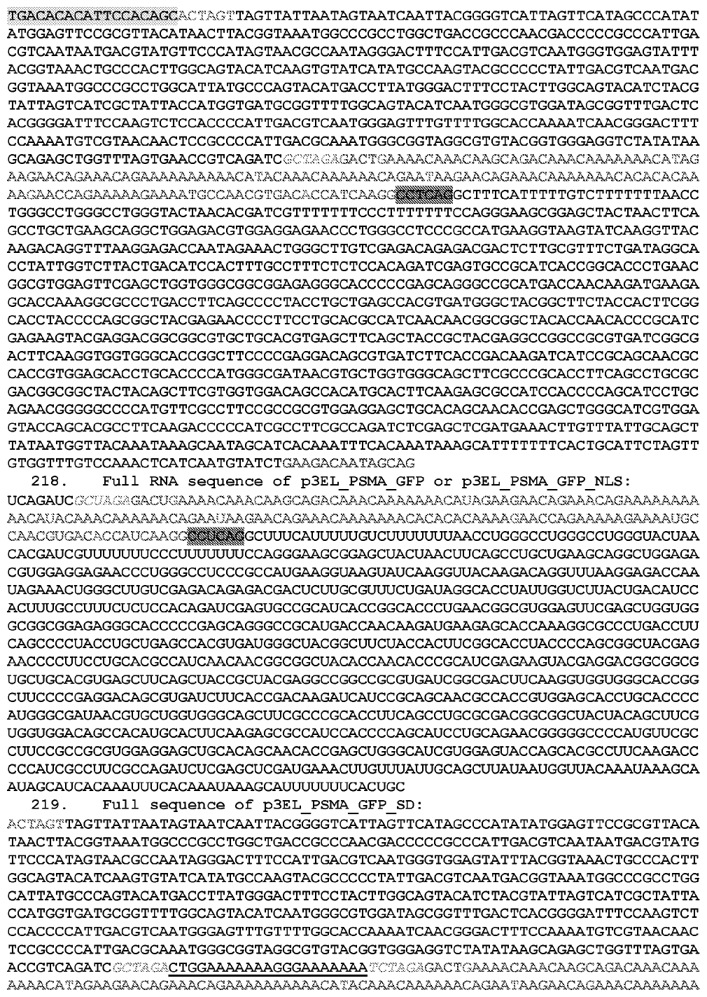

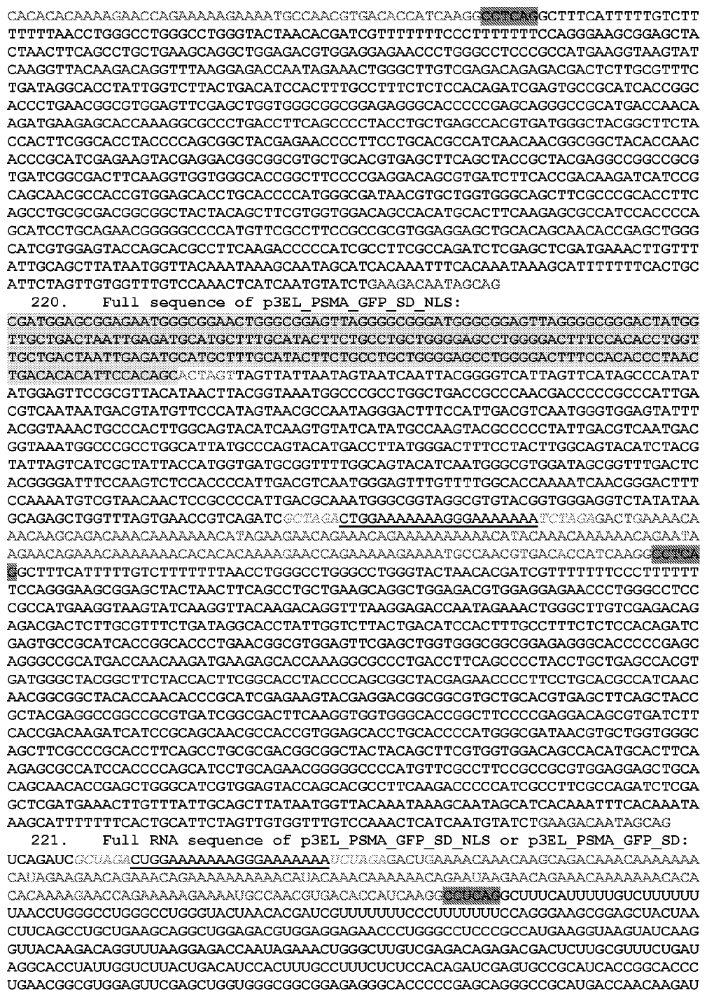

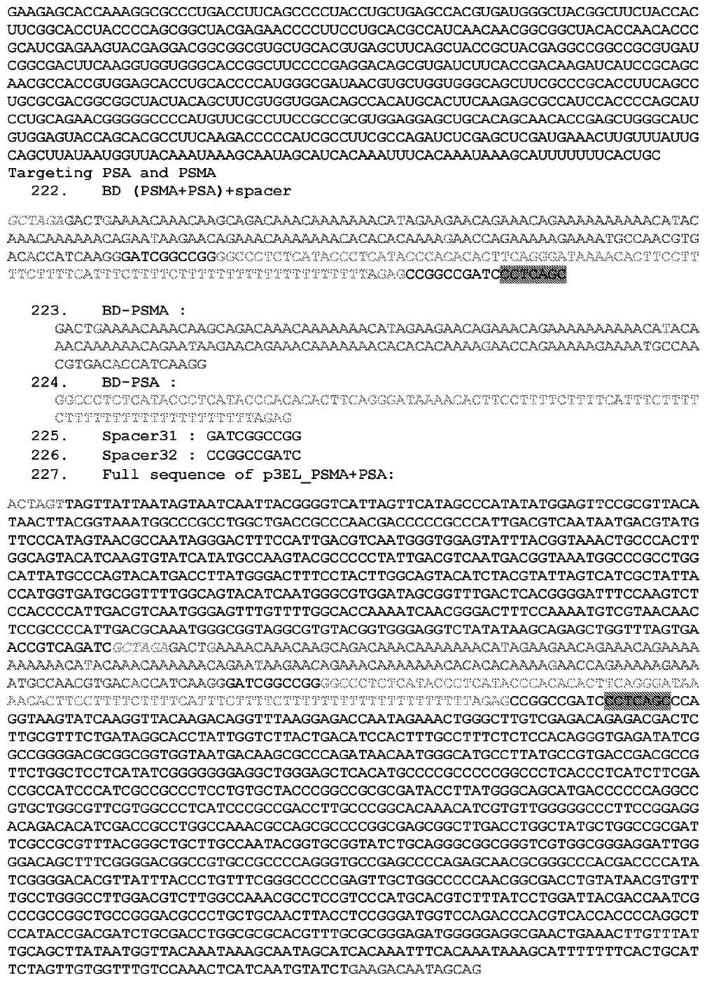

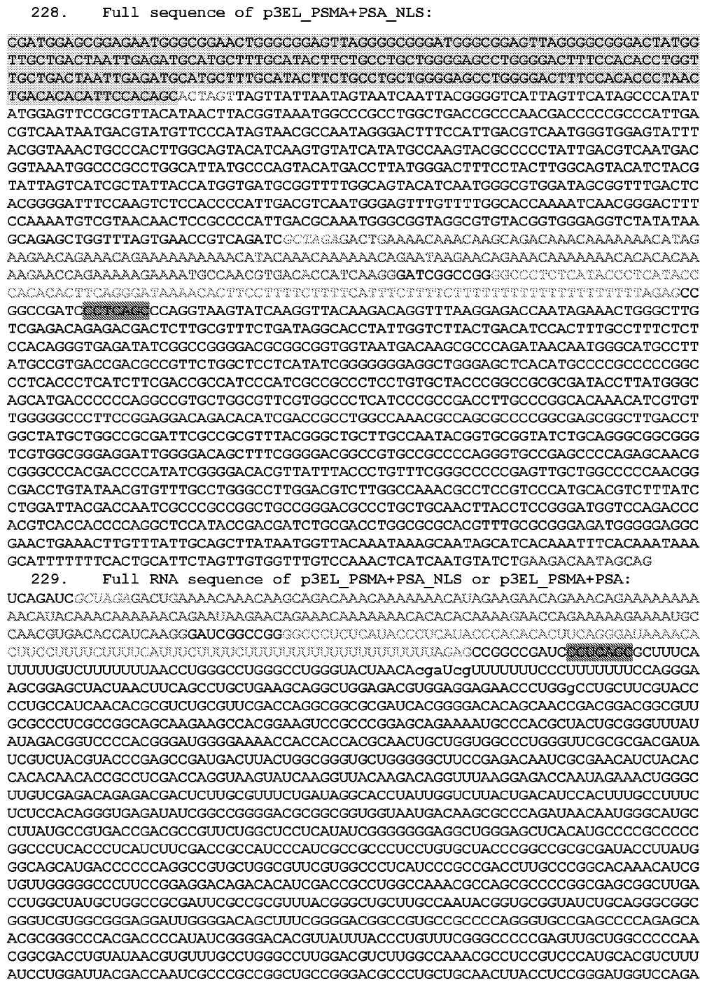

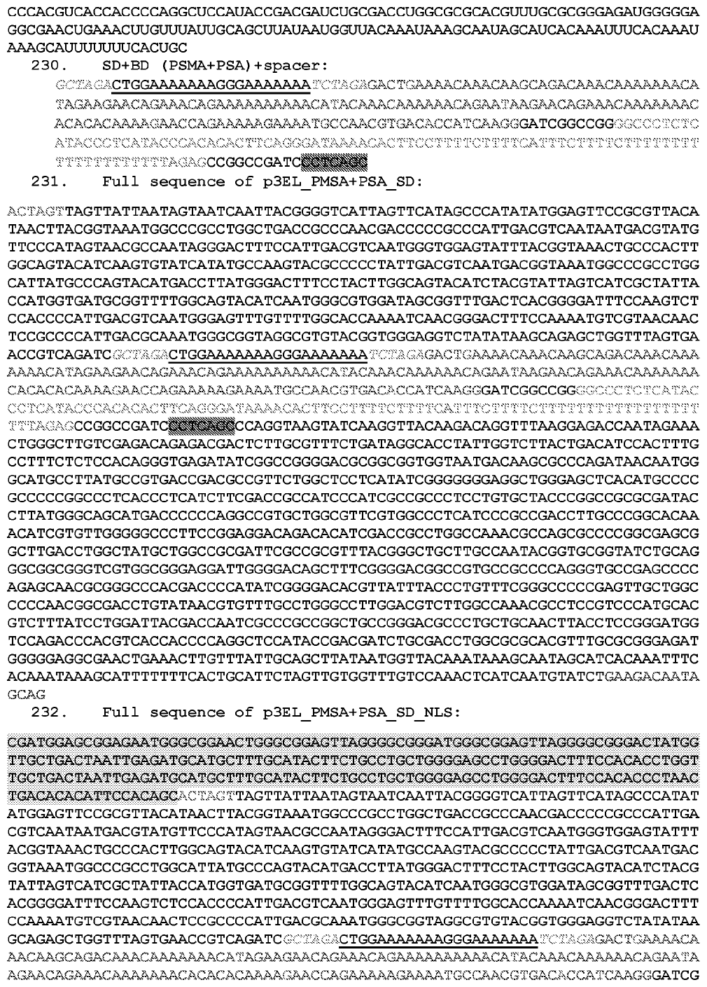

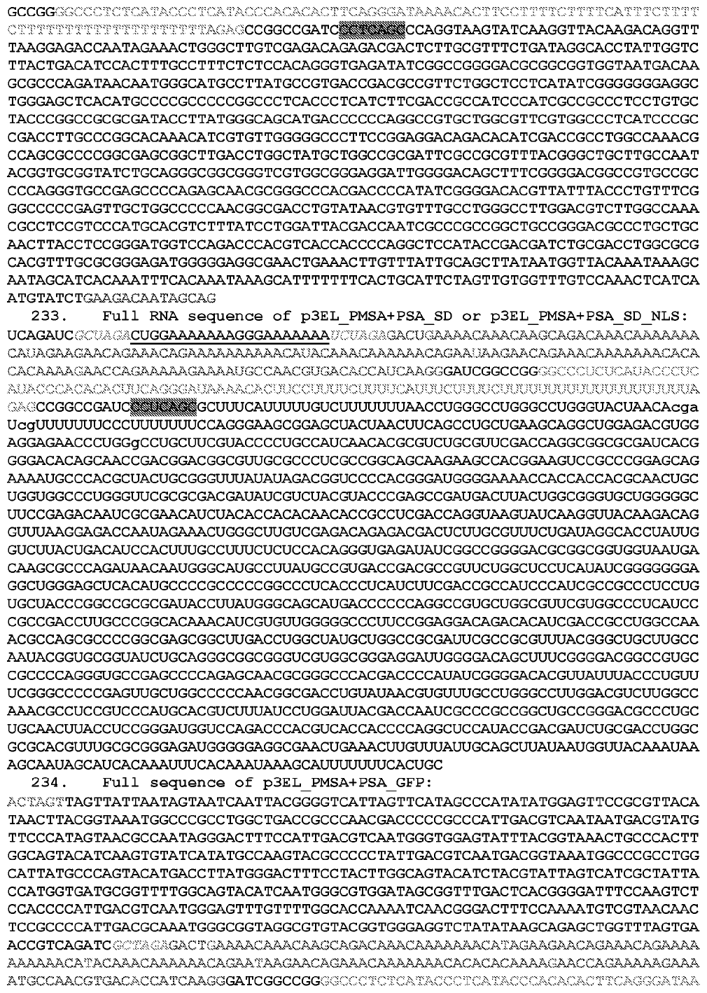

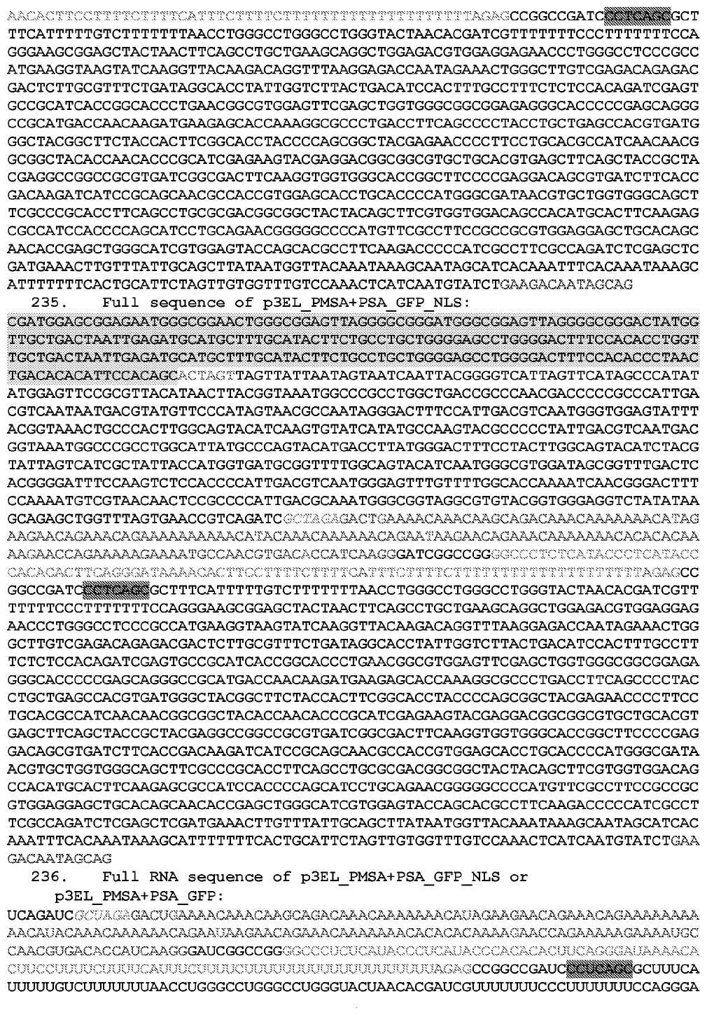

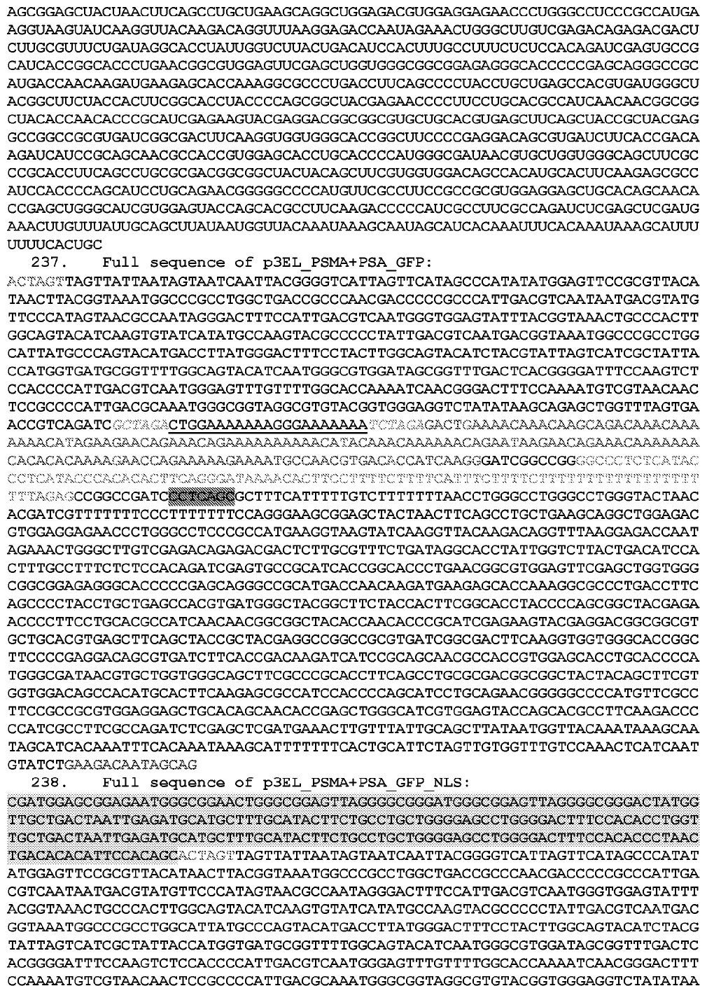

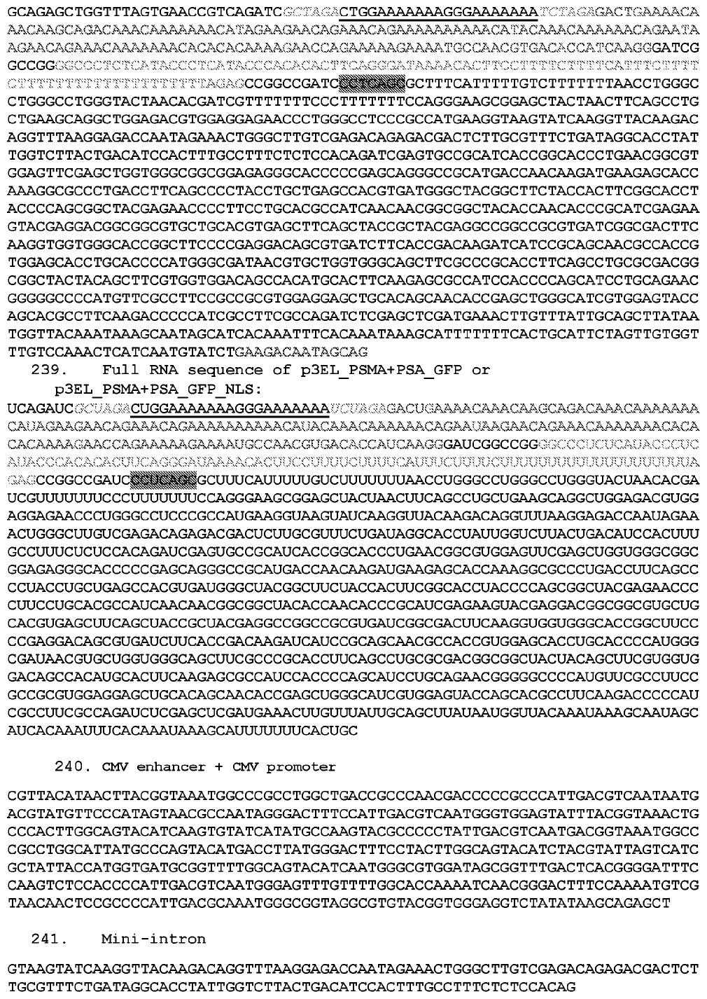

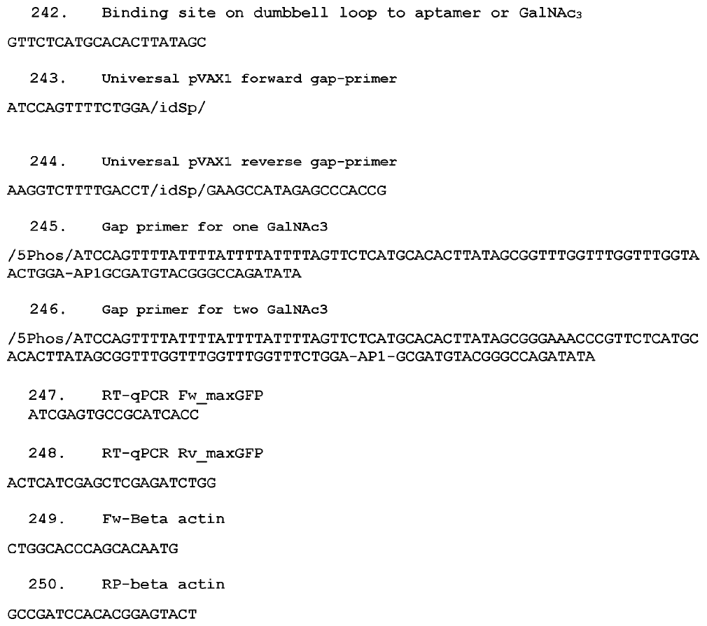

FIG. 20. Shows the Sequence Listing (all sequences from 5′ to 3′) of the constructs used in this invention. Sequences 57-68 show constructs including a safety domain, multiple binding domains and an optional nuclear localization signal.

### METHODS

### Methods and Materials

### RNA Design:

The trans-splicing constructs were designed combining various reported and novel molecular features to improve activity and target specificity. The 3′ER ts constructs consisted of a CMV promoter (pEGFP-N1, Clontech acc no. U55762) followed by a binding domain (BD) of 50 bases complementary to the target AFP intron 5. The BD included two mismatches at positions 18 and 19 to inhibit potential antisense (as) effects that can be triggered by longer dsRNA in the nucleus of the cell. Software ‘foldanalyze’ (HUSAR, DKFZ) was used to select short unstructured BDs within the complete antisense RNA structure space that can be directed against the AFP intron 5. Structures of the selected BDs were confirmed by RNA 2° structure (minimum free energy and centroid) predictions using software tools mfold and RNAfold. Such selected BDs were then fused with the rest of the trans-splicing RNA making sure that the BDs remained unstructured upon fusion and were not involved in base-pairing the trans-splicing or coding domains which was achieved by implementing suitable spacers. The selected 3′ splice signal (3′ss) was designed to functionally compete with the cellular cis-splice site and was supported by an intronic splice enhancer (ISE) (McCarthy, et al., 1998; Konczak, et al., 2000; Yeo et al., 2004), a branchpoint (BP) (Eul, 2006) and polypyrimidine tract (Ppt) (Nobel, et al., 1998; Taggart, et al, 2012). The HSVtk cds was preceded with a sequence coding for a proteolytic cleavage site P2A (Kim, et al, 2011) to ensure endogenous release of the native HSV-tk from the AFP-HSVtk fusion protein that initially results from the trans-splicing process. The HSVtk gene is devoid of a start codon and can only be translated after trans-splicing using the translational start of the target message. The HSVtk gene was equipped with an A/G-rich exonic splice enhancer (ESE) generated by using degenerative alternative codons that do not alter the HSV-tk amino acid sequence (Fairbrother, et al, 2002; Jin et al., 2003) (FIG. 1). A beta-globin mini-intron of 133 bases (pCMVTNT™, acc num. AF477200.1) was introduced in the HSVtk gene at a splice site consensus motif (3′ ss CAG/G and 5′ss MAG. For transcriptional termination the SV40 polyA sequence (pcDNA3.1, Life Technologies) was used.

The 5′ER ts constructs were designed with the same molecular features as p3ER but with different orientation including a translational signal motif along with the CMV promoter. All the structural elements important for translation of eukaryotic mRNA were included: original cap site of AFP (Gibbs, et al. 1987) followed by the consensus Kozak sequence GCCRGCCAUGG (Kozak, 1995, 1999, 2005). Immediately after the translation start signal was the coding domain HSVtk inclusive of the ESE and mini-intron followed by a 5′ss signal (Freund, et al. 2005). The 5′ BD was designed in a similar way with mismatches at positions 24 and 25 to avoid antisense effects a (s-effects). Following the BD, a hammerhead ribozyme (HH Rz) (Saksmerprome, et al. 2004) was incorporated for enhanced cleaving of the BD after delivery into the nucleus. The HH RZ is followed by a long spacer to isolate the polyA from the ribozyme followed by the SV40 polyA.

### Prediction of Splice Sites:

The strength and nature of the splice sites were predicted using softwares Alternative Splice Site Predictor (ASSP) (Wang, 2006) http://wangcomputing.com/assp/overview.html and Berkeley Drosophila Genome Project Splice Site Prediction (BDGP SSP) (Reese, et al., 1997) http://www.fruitfly.org/seqtools/splice.html using default cut-off values for the splice site predictions. To predict the nature of splice sites in HPV16 genome, ASSP was used to document constitutive or cryptic splice acceptors and donors based on the overall score and confidence generated by the software. The alternative splice sites with confidence >0.89 and score >5.5 and constitutive splice sites with confidence >0.1 and score >7.7 besides the documented splice sites (Johansson, 2013 and Schmitt, et al, 2011) were selected for the trans-splicing analyses.

### Plasmid Construction

Plasmid pGFP was cloned by inserting the maxGFP gene (CMV promoter, cds, SV40 ployA site) of the pMAX-GFP (Lonza) vector into the NdeI and BbsI restriction endonuclease cleavage sites of the pVAX1 vector (Addgene). The SV40 enhancer which functions as DNA nuclear localizing signal (dNLS) was inserted into the NdeI and BbsI site upstream of the CMV promoter. The HSVtk positive control plasmid carried a codon optimized HSV1 thymidine kinase coding sequence inserted into the pVAX1 plasmid under the control of the CMV promoter.

### Dumbbell (db) Construction:

Generating dumbbells for trans-splicing from the plasmid vectors was done using the ELAN method of db production. The Enzymatic Ligation Assisted by Nucleases (ELAN) is a three-step process which includes digestion of the transcription cassette from the plasmid, ligation of the closing loops on either side followed by exonuclease treatment to eliminate the unclosed db plasmids.

### (a) Phosphorylation of Stem-Loop Primers

The stem loops consisting of individual RE site were synthesised by AIT Biotech (Singapore) and was phosphorylated using the following reaction shown in Table 1:

TABLE 1
Reaction setup for stem-loop phosphorylation using
polynucleotide kinase (PNK)

| COMPONENTS | STEM LOOP PRIMERS |
|---|---|
| Stem loop oligo 10 μM | 60 pmoles |
| 10X Buffer A | 2 μL |
| PNK enzyme | 1-2 U |
| 10 mM ATP | 2 μL |
| Water nuclease-free | Make up volume |
| TOTAL | 20 uL |

The Stem-loop primers were Stem loop-SpeI and Stem-loop-BamHI.

### (b) ELAN Method

In the ELAN loop-ligation method, the gene expression cassette was directly cut out from parental plasmid. 50 times more stem-loops were added in the ligation reaction to ensure that most of the gene expressing cassettes could be capped. By-products such as loop dimers were cleaved by the restriction enzymes and were destroyed during the exonuclease treatment. Detailed setups of the reaction are shown in Table 2.

TABLE 2
Reaction setup for the generation of trans-splicing dumbbells
using the ELAN loop-ligation strategy

| COMPONENTS | AMOUNT | CONDITIONS |
|---|---|---|
| Digestion | | |
| Parental plasmid | 6 pmoles | 37° C. incubation for 4 hours |
| | | and heat inactivation at |
| | | 65° C. for 15 minutes |
| Spel RE | 5 U | |
| BamHI RE | 5 U | |
| HindIII RE | 5 U | |
| 10x Fast digest buffer | 5 μL | |
| Water nuclease-free | Make up volume | |
| TOTAL | 50 μL | |
| ELAN reaction | | |
| Digestion mix | 50 μL | 22° C. for 4 hours to overnight |
| | | and heat inactivation at |
| | | 85° C. for 5 min |
| Loop-1 | 60 pmoles | |
| Loop-2 | 60 pmoles | |
| 10X Fast digest buffer | 10 μL | |
| 100 mM ATP | 1.5 μL | |
| Spel RE | 1 U | |
| BamHI RE | 1 U | |
| BgIII RE | 1 U | |
| HindIII RE | 1 U | |
| Xbal RE | 1 U | |
| T4 DNA ligase | 3 U | |
| Water nuclease-free | Make up volume | |
| TOTAL | 15 μL | |
| Exonuclease treatment | | |
| ELAN mix | 148 μL | 37° C. incubation for 2 hours |
| | | and heat inactivation at |
| | | 85° C. for 5 min |
| T7 DNA polymerase | 10 U | |

The generated dumbbells were run on a 1% agarose gel to confirm their integrity.

### Gap-Primer PCR

Alternatively to the ELAN method, dumbbells were produced using gap-primer PCR (gpPCR).
1. Optional: Linearize the plasmid DNA template by restriction endonuclease cleavage either upstream or downstream of the PCR amplicon. 100 ng of plasmid template DNA were linearised using FastDigest BclI and FastDigest DraI endonuclease enzymes during a 2 h incubation in 10× FastDigest buffer and a reaction volume of 100 μL.
2. Prepare a 10 μM working stock of gap-primers and a dNTP mix (10 mM each) using nuclease-free water.

Universal pVAX1 forward gap-primer, 100 μM (PAGE purified):
(SEQ ID NO: 243)
5′-pATCCAGTTTTCTGGA/idSp/GACTCTTCGCGATGTACGGG-3′
Universal pVAX1 reverse gap-primers, 100 μM (PAGE purified):
(SEQ ID NO: 244)
5′-pAAGGTCTTTTGACCT/idSp/GAAGCCATAGAGCCCACCG-3′
gap-primer for one GalNAc3:
(SEQ ID NO: 245)
5′-/5Phos/ATCCAGTTTTATTTTATTTTATTTTAGTTCTCATGCACACTTATAGCGGTTTGGTTT
GGTTTGGTAACTGGA/idSp/GCGATGTACGGGCCAGATATA-3′
gap-primer for two GalNAc3:
(SEQ ID NO: 246)
5′-/5Phos/ATCCAGTTTTATTTTATTTTATTTTAGTTCTCATGCACACTTATAGCGGGAAACCC
GTTCTCATGCACACTTATAGCGGTTTGGTTTGGTTTGGTTTCTGGA/idSp/GCGATGTACG
GGCCAGATATA-3′.

3. For a 100 μL reaction, mix 10 μL of Q5® Reaction Buffer, 5 μL of Taq polymerase Reaction Buffer, 5 μL of MgSO4, 2 μL of 10 mM dNTP mix, 4 μL of 10 μM forward gap-primer, 4 μL of 10 μM reverse gap-primer, 0.1 μL (0.5 U) of Taq DNA polymerase, 0.1 μL (1 U) of Q5® DNA polymerase, and 10 ng of DNA template in a 0.2 mL PCR tube, then top up with water to a volume of 100 μL.
4. Pulse down and subject the mix to PCR by an initial denaturation of 98° C. for 30 sec, followed by 25 to 30 cycles each consisting of a denaturation step at 98° C. for 10 sec, and primer annealing step at 64° C. for 30 sec, and the primer extension at 72° C. for 30 sec/kb, and a final extension at 72° C. for 2 min.
5. Load 5 μL of reaction with 1 μL of 6× loading dye onto 1% agarose gel and run at 100 V for 1 h to verify the PCR product.

### Ligation of Gp-Dumbbells

1. For a 150 μL ligation reaction, mix the 100 μL PCR reaction in the 0.2 mL PCR tube with 15 μL of 10× T4 DNA ligase buffer, 5 μL (20 U) of T4 DNA ligase and 30 μL of nuclease-free water and spin down.
2. Incubate at room temperature (20° C.-25° C.) for 1 h.

### Optional: Exonuclease Treatment

3. Aliquot 5 μL of ligation reaction into a fresh 0.2 mL PCR tube for DNA gel electrophoresis.
4. Add 5 μL of T7 DNA polymerase to the ligation reaction and incubate at 37° C. for 30 min. Then heat inactivate the ligase at 85° C. for 5 min.
5. Load 5 μL of untreated sample and 5.2 μL of exonuclease-treated sample on 1% agarose gel. Run the gel at 100 V for 1 h. Quantify the conversion rate by quantifying and comparing the band intensities of the exonuclease-treated sample versus untreated sample.
6. Proceed with purification using QIAquick PCR purification kit. Add 5 times the reaction volume of PB binding buffer, mix well and transfer into the QIAquick spin column. Centrifuge at 12,000 g for 1 min. Discard the flow through. Add 700 μL of PE wash buffer to the spin column and centrifuge at 12,000 g for 1 min. Discard the flow through before dry spin at 12,000 g for 1 min. Transfer the spin column to a new 1.5 mL microcentrifuge tube and add 50 μL of nuclease-free water for elution. Spin at 12,000 g for 1 min.
7. Larger volumes of the ligation reaction can be purified via phenol-chloroform-isoamylalcohol (PCI) (25:24:1) extraction, followed by three chloroform-isoamylalcohol (CI) (24:1) re-extractions, and ethanol precipitation. Therefore, an equal volume of PCI is added to the aqueous DNA containing phase and vortexed at maximum speed for 2 min followed by 10 min centrifugation at 12,000 g. The upper aqueous phase is then transferred to a new tube and re-extracted 3 times with equal volumes of CI. For the re-extractions, the mixtures are intensively hand-shaken, not vortexed, for 30 sec before separating the phases by centrifugation (12,000 g, 30 sec). After the third re-extraction, the upper aqueous phase is transferred to a fresh tube, supplemented with 0.1 volumes of 3 M ammonia, sodium or potassium acetate (pH 4.8-5.2) and 2.5 volumes of absolute ethanol (4° C.). The solution is gently mixed, incubated at −20° C. for 20 min or longer, and the dumbbell DNA is pelleted by centrifugation (15 min, 4° C., 12,000 g). The supernatant is discarded, the pellet is washed with 70% ethanol (4° C.), and dried (air dried or using a speedvac centrifuge).

### Production of Superior Dumbbell Vectors

At high purity using a process termed 1-2-3 gap-primer PCR. This process represents a 1-tube, 2-enzyme, 3 h procedure that comprises a PCR followed by a ligation. The resulting dumbbells harbor mismatches close to the loop structures, which facilitate nuclear diffusion and result in enhanced gene expression.

Wolfgang Walther (ed.), Gene Therapy of Cancer: Methods and Protocols, Methods in Molecular Biology, vol. 2521, https://doi.org/10.1007/978-1-0716-2441-8 18, Springer Science+Business Media, LLC, part of Springer Nature 2022.

Carry out all procedures at room temperature unless otherwise specified.

### Gap-Primer PCR 1.

Universal pVAX1 forward gap-primer, 100 μM (PAGE purified):
SEQ ID No: 243
5′-pATCCAGTTTTCTGGA/idSp/GACTCTTCGCGATGTACGGG-3′
Universal pVAX1 reverse gap-primers, 100 μM (PAGE purified):
SEQ ID No: 244
5′-pAAGGTCTTTTGACCT/idSp/GAAGCCATAGAGCCCACCG-3′

The gap-PCR primers harbor an invariable universal 5′ domain comprising a 5′ phosphate, a stretch designed to refold and preform the dumbbell loop, the abasic position, and a 3′ domain that is binding toward the DNA template (underlined). In this example, the 3′ ends are complementary to the cloning vector pVAX1
1. Optional: Linearize the plasmid DNA template by restriction endonuclease cleavage either upstream or downstream of the PCR amplicon. 100 ng of plasmid template DNA were linearized using FastDigest BclI and FastDigest DraI endonuclease enzymes during a 2 h incubation in 10× FastDigest buffer and a reaction volume of 100 UL. Linearization of DNA template using endonuclease digestion increases the PCR yields
2. Prepare a 10 μM working stock of gap-primers and a dNTP mix (10 mM each) using nuclease-free water.
3. For a 100 μL reaction, mix 10 μL of Q5® Reaction Buffer, 5 μL of Taq polymerase Reaction Buffer, 5 μL of MgSO4, 2 μL of 10 mM dNTP mix, 4 μL of 10 μM forward gap-primer, 4 μl of 10 μM reverse gap-primer (see Note 3) (FIG. 3), 0.1 μL (0.5 U) of Taq DNA polymerase (see Notes 4 and 5), 0.5 μL (1 U) of Q5® DNA polymerase, and 10 ng of DNA template in a 0.2 mL PCR tube, then top up with water to a volume of 100 μL.
4. Pulse down and subject the mix to PCR by an initial denaturation of 98 C for 30 s, followed by 25-30 cycles (see Note 6) each consisting of a denaturation step at 98 C for 10 s (FIG. 6), an primer annealing step at 64 C for 30 s (see Note 7), and the primer extension at 72 C for 30 s/kb, and a final extension at 72 C for 2 min.
5. Load 5 μL of reaction with 1 μL of 6× loading dye onto 1% agarose gel and run at 100 V for 1 h to verify the PCR product.

### 3.2 Ligation

1. For a 150 μL ligation reaction, mix the 100 μL PCR reaction in the 0.2 mL PCR tube with 15 μL of 10× T4 DNA ligase buffer, 5 μL (20 U) of T4 DNA ligase, and 30 μL of nuclease-free water and spin down.
2. Incubate at room temperature (20-25 C) for 1 h. Higher ligation efficiency can be achieved by incubating the ligation reaction overnight at room temperature.

### 3.3 Optional:

### Exonuclease Treatment

1. Aliquot 5 μL of ligation reaction into a fresh 0.2 mL PCR tube for DNA gel electrophoresis.
2. Add 5 μL of T7 DNA polymerase to the remaining 145 μL ligation reaction and incubate at 37° C. for 30 min. Then heat inactivate the ligase at 85° C. for 5 min.
3. Load 5 μL of untreated sample (step 1) and 5.2 μL of exonuclease-treated sample (step 2) on 1% agarose gel. Run the gel at 100 V for 1 h. Quantify the conversion rate by quantifying and comparing the band intensities of the exonuclease-treated sample versus untreated sample (FIG. 7).
4. Proceed with purification using QIAquick PCR purification kit.

Add 5 times the reaction volume of PB binding buffer, mix well and transfer into the QIAquick spin column. Centrifuge at 12,000×g for 1 min. Discard the flowthrough. Add 700 μL of PE wash buffer to the spin column and centrifuge at 12,000×g for 1 min. Discard the flowthrough before dry spin at 12,000×g for 1 min. Transfer the spin column to a new 1.5 ml microcentrifuge tube and add 50 μL of nuclease-free water for elution. Spin at 12,000×g for 1 min.
5. Larger volumes of the ligation reaction can be purified via phenol-chloroform-isoamyl alcohol (PCI) (25:24:1) extraction, followed by three chloroform-isoamyl alcohol (CI) (24:1) re-extractions, and ethanol precipitation. Therefore, an equal volume of PCI is added to the aqueous DNA containing phase and vortexed at maximum speed for 2 min followed by 10 min centrifugation at 12,000×g. The upper aqueous phase is then transferred to a new tube and re-extracted 3 times with equal volumes of CI. For the re-extractions, the mixtures are intensively hand-shaken, not vortexed, for 30 s before separating the phases by centrifugation (12,000×g, 30 s). After the third re-extraction, the upper aqueous phase is transferred to a fresh tube, supplemented with 0.1 volumes of 3 M ammonia, sodium or potassium acetate (pH 4.8-5.2) and 2.5 volumes of absolute ethanol (4° C.). The solution is gently mixed, incubated at 20 C for 20 min or longer, and the dumbbell DNA is pelleted by centrifugation (15 min, 4 C, 12,000×g). The supernatant is discarded, the pellet is washed with 70% ethanol (4° C.), and dried (air dried or using a speedvac centrifuge).

### Formation of Dumbbell Conjugates

Dumbbell-GalNAc3-conjugates

3.5 pmol GalNAc3-DNA or GalNAc3-RNA oligonucleotide was annealed with 3.5 pmol dumbbell DNA in 20 μl 10× hybridisation buffer (1 M NaCl, 0.1 M MgCl2, 200 mM Tris-HCl, pH 7.4) in the presence of 20% v/v of PEG4000. The solution was denatured at 80° C. for 5 min and then incubated at 37° C. for 1 hour. The resulting dumbbell-GalNAc3 conjugates were cleaved with AseI (Thermo Fisher) and GalNAc3 attachment to the conjugation loops was monitored in 1.5% agarose gel shift assays. Dumbbell-conjugates were purified using Sephadex gel permeation chromatography and ethanol precipitation.

GalNAc3-linked oligonucleotides GalNAc3-DNA 5′-GCTATAAGTGTGCATGAGAAC-GalNAc3-3′ and GalNAc-RNA 5′-GCUAUAAGUGUGCAUGAGAAC-GalNAc3-3′ were derived from Microsynth (Switzerland). Underlined positions indicate deoxyribonucletides. Primers for the production of dumbbells were derived from IDT.

Dumbbell-aptCD137-2-conjugates

3.5 pmol of the aptCD137-2 homodimer was annealed with 3.5 pmol dumbbell DNA as described above.

### Cell Culture:

Human tissue culture cells including HepG2, HEK293T, HONE-1 and C666 were maintained at 37° C. in a humidified incubator with 5% CO2 in Dulbecco's Modified Eagle's Medium (HyClone, Thermo Scientific), supplemented with 10% Fetal Bovine Serum (HyClone) and 1% penicillin-streptomycin. The cells were passaged every 3-4 days at desired density.
   Transfection of Human Tissue Culture Cells with DNA Vectors:

Cells were transfected with plasmids or dumbbell-shaped DNA minimal vectors using Lipofectamine® 3000 following the manufacturer's protocol. In short, 500 ng of DNA and 1 μl of P3000 were diluted in 25 μl of Opti-MEM and then mixed with 1.5 μl Lipofectamine 3000 (diluted in 25 μl of Opti-MEM). The mixture was incubated at room temperature for 5 minutes before adding onto the cells.

### Total RNA Isolation:

RNA was isolated 24 hours post-transfection using RNeasy plus kit (Qiagen) following the manufacturer's protocol. RNA concentrations were measured using NanoDrop 2000.

cDNA Conversion and Real-Time RT-PCR:

500 ng RNA from all samples was converted into cDNA using the First Strand SuperScript RTIII (Invitrogen) kit with 200 ng of random hexamers and 10 μM of dNTPs. The reaction conditions were 25° C. for 5 min, followed by 50° C. for 2 h and enzyme inactivation at 70° C. for 15 min. 20 ng of cDNA was used as template for real time RT-PCR. TaqMan quantification was performed in ABI 7900HT of the cDNAs by designing specific probe and primer sets for each cis- and trans-splicing detection. The number of cycles in over-expression studies and endogenous studies were 40 and 50 respectively. RT-PCR: Reverse transcription PCR was performed on the cDNA samples using Taq DNA polymerase (Fermentas) with 60 cycles of two-step PCR (30+30 cycles or 35+35 cycles) to detect 3′ and 5′ cis and trans-splicing and the bands were visualized on a 1% agarose gel.

For RT-qPCR, Fw_maxGFP (5′-ATCGAGTGCCGCATCACC-3′) SEQ ID No:247 and Rv_maxGFP (5′-ACTCATCGAGCTCGAGATCTGG-3′) SEQ ID No:248 were used. Fw-Beta actin (5′-CTGGCACCCAGCACAATG-3′) SEQ ID No:249 and RP-beta actin (5′-GCCGATCCACACGGAGTACT-3′) SEQ ID No:250 were used as housekeeping gene.

Uptake of Dumbbell-GalNAc3-Conjugates from the Tissue Cell Culture Medium
Uptake of MaxGFP dumbbell-GalNAc3-conjugates

HepG2 cells were trypsinized, washed with 10 ml DMEM, 0.05×10⁶ cells were resuspended in 30 μl DMEM and 3.5 pmol MaxGFP dumbbell-GalNAc3-conjugates dissolved in 20 μl of water were added and incubated with the cells for 4 hour before seeding them again. By adding the dumbbell conjugates to HepG2 cells in suspension, we achieved a higher concentration of dumbbell conjugates in the medium to observe stronger expression of MaxGFP.

### Uptake of HSVtk Dumbbell-GalNAc3 or -2GalNAc3-Conjugates

0.05×10⁶ HepG2 cells were seeded in 24 wells and 0.35 pmol HSVtk dumbbell-GalNAc3 or -2GalNAc3-conjugates dissolved in 20 μl of water were added after 24 hours to the DMEM culture medium.

qPCR Quantification of Uptaken MaxGFP Dumbbell-GalNAc3 DNA

After 24 hours of exposure, cells were harvested and episomal nucleic acids including uptaken dumbbell-conjugates were isolated using the RNeasy® Plus kit following the manufacturer's protocol. For SYBR Green detection of dumbbell DNA, 1 μl of the episomal nucleic acid sample was mixed with 1×SYBR® Select Master Mix for CFX and each 0.5 μM forward and reverse primers in a 10 μl reaction. All reactions were run in duplicate. Dumbbell vector DNA was quantified using absolute qPCR quantification based on a standard curve created with dumbbell vector DNA.

### AlamarBlue Assay:

To check the functional activity of trans-splicing, drug Ganciclovir (GCV) (Sigma) was added to the cells at a concentration of 10 μM, 100 μM and no GCV (internal negative control) 24 hours post-transfection followed by addition of AlamarBlue® cell viability reagent (Thermo Scientific) 24 hours post drug for a duration of 6 days with replacement of fresh media and drug every day after each alamarblue reading. The fluorescence was measured at 230/290 nm after 90 minutes of incubation at 37° C. The positive and negative controls for the assay were designed as mentioned in the manufacturer's protocol.

### Flow Cytometry:

For FACS analysis, the media was aspirated, and the cells were rinsed once with PBS before trypsinisation with 200 μl of 1× trypsin-EDTA. The trypsinised cells were collected by centrifugation at 4200 rpm for 6 min in 1 ml of media. The pelleted cells were resuspended in 500 μl of 1×PBS. FACS was performed with 10,000 cells for the NTC and >5,000 cells per sample using a LSRFortessa cell analyser, and FACSDiva software v6.1.3 was used for the acquisition of the samples. FlowJo software V7.6.1 was used for data analysis.

To check for apoptosis, cells were harvested 48 hours post 100 μM GCV treatment and stained using Propidium Iodide and Alexa Fluor 647 Annexin V (Life Technologies) in Annexin-binding buffer according to manufacturer's protocol. The samples were gated based on single live cell populations which were positive for GFP. The final % apoptosis values are indicated as (early and late apoptosis+GCV)-(early and late apoptosis−GCV).

### Preparation of Ganciclovir Working Stock

GCV was purchased from Sigma as a 100 mg ready-to-mix powder form. To dissolve the powder for master stock for cell culture experiments, 10 mg of GCV was dissolved in 1 ml of 0.1 N/0.1 M HCl (final concentration: 10 mg/ml). For a 10 mM working stock (for conditions ranging from 1-100 μM), 255 μl of 10 mg/ml GCV master stock was diluted in 745 μl of 0.1 N HCl. For 1 mM working stock (for conditions ranging from 0.1 μM and 0.3 μM), 100 μl of 10 mM working stock was further diluted in 900 μl of 0.1 N HCl.

### AlamarBlue® Cell Viability Assay

To trigger death of dumbbell-conjugate transfected HepG2 cells, GCV was added to the cells at a concentration of 100 μM, 24 hours post-transfection. The cell death activity was monitored using the AlamarBlue® cell viability reagent every 24 hours for 6 days with addition of fresh media and drug every day. The fluorescence was measured at 530 nm/590 nm after 90 min of incubation.

### Tail Vein Injection of Mice

For tail vein injection, 30 μg of dumbbell-Nanoluc and dumbbell-maxGFP was prepared in 100 μl solution with 0.14 μl/μg DNA of in vivo-JetPEI (Polyplus), SAINT-Vivo (Synvolux) or PBS respectively. 30 μg of dumbbell-Nanoluc/maxGFP-RNA-1-GalNac3 was diluted in 100 μl PBS. For hydrodynamic injection, 30 μg of dumbbell-Nanoluc and dumbbell-maxGFP was diluted in 1.5 mL of PBS. Samples were administered to 4-6 weeks old mice by iv injection. Organs were harvested at day 6 and homogenization of organs was performed prior to RNA isolation with TRIzol. cDNA was converted from 5 μl of RNA and qPCR was carried out.

### Statistical Analysis

Error bars represent standard errors of the arithmetic mean (±SEM) of three independent experiments. Unpaired student's t-test was used to determine significance when comparing two groups. GraphPad Prism 9 software was used for the statistical analysis. P values are as indicated on the graphs.

### Results and Discussion

In detail, this invention refers to
(i) the design of trans-splicing-based RNAs, ideally multi-targeting, and so targeting disease-specific or/plus housekeeping gene derived pre-mRNAs for enhanced activity,
(ii) novel highly active multi-targeting trans-splicing-based RNAs for new targets,
(iii) dumbbell-shaped DNA delivery vectors that are featured with helper functions such as aptamers or tri-antennary GalNAc residues for targeted delivery into a variety of cell types, including hepatocytes, CD137+ cells, and PSMA+ cells, and
(iv) the use of multiple helper functions, such as dumbbell-GalNAc3 conjugates with two or more GalNAc3 residues (attached via two antisense oligonucleotide binding sites resulted in more positive results (80.4%).

This invention concerns novel optimized RNA sequences and structures designed to achieve higher trans-splicing activity and specificity. We designed parental trans-splicing RNA (tsRNA) molecules for 3′ exon labelling comprising some or all of the following molecular features (FIG. 1): Firstly, one or multiple computationally selected unstructured binding domains (BD) of 25 to 250 nt in length complementary to pre-mRNA targets and, ideally, a spacer preserving the selected BD structure in the context of the tsRNA molecule; secondly, a splicing signal or domain composed of an intronic splice enhancer (ISE), a consensus branch point (BP) sequence, an extensive polypyrimidine tract (PPT), and a consensus splice acceptor (SA) site (AG/G); and thirdly, a coding domain including, for example, the HSVtk gene harbouring a strengthened exonic splice enhancer (ESE) as well as the β-globin mini-intron (FIG. 1). The tsRNA are, ideally, further equipped with the P2A proteolytic cleavage site positioned immediately downstream of the SA site to trigger proteolytic release of the HSVtk from the chimeric fusion protein which results from the trans-splice reaction. Target mismatches are included into the binding domains (ΔBD) to avoid that target binding generates long double-stranded nuclear RNA which might trigger antisense effects, including A-to-I editing by adenosine deaminases acting on RNA (ADARs), which could impair the trans-splice strategy. The trans-splicing RNAs are characterised by a safety domain (SD) that binds towards its own splice acceptor to suppress off-target trans-splicing. In addition, some of the constructs are being featured with a DNA nuclear import sequence (NLS) (FIG. 1).

Upon cellular delivery, in one embodiment, the trans-splicing based suicide RNAs will bind towards the respective pre-mRNA targets, splice in trans, and enable target cell-specific expression of the HSVtk. Upon co-delivery, the prodrug ganciclovir (GCV) is being phosphorylated by the HSVtk with support from cellular phosphatases triggering the formation of the toxic GCV-triphosphate which leads to cell death (Poddar et al., 2018).

A novel design aspect is the generation of multi-targeting trans-splicing-based suicide RNAs targeting disease-specific plus housekeeping gene derived pre-mRNAs. As opposed to the disease-specific pre-mRNA biomarkers, pre-mRNAs derived from housekeeping genes are constitutively expressed in all cell types. The housekeeping genes include but are not limited to genes involved in gene expression, metabolism, cellular structure, cellular surfaces, signalling, and others. This novel design applies to target cells in which the disease-specific biomarkers are very limited by numbers and/or the level of expression. In such a scenario, trans-splicing towards a housekeeping sequence can trigger basal expression of the death signal but without yet killing the cells. Only additional trans-splicing towards a disease or cell type-specific pre-mRNA biomarker will elevate the expression of the death signal above the threshold that finally kills the cell.

Multi-Targeting Trans-Splicing-Based Suicide RNAs Trigger Superior Levels of Cell Death at 300-Fold Lower GCV Concentration as Compared with Single-Targeting Suicide RNAs

Another embodiment of this invention are trans-splicing-based suicide RNAs targeting hepatoblastoma-derived cells harbouring target binding domains which are complementary to alpha-fetoprotein (AFP), Vascular endothelium growth factor (VEGF), γ-glutamyl transferase (GGT), Hepatocellular carcinoma associated protein 2 (HCCA2), Transforming growth factor beta 1 (TGF-β1), cluster of differentiation 24 (CD24), Cyclin D1 (CCND1), Glypican 3 (GPC3) and Telomerase reverse transcriptase (TERT). Such multi-targeting suicide RNAs triggered superior cell death activity on hepatoblastoma-derived human cells at 300-fold lower GCV concentrations as compared with single-targeting suicide RNAs (FIG. 2). A suicide RNA targeting five pre-mRNA biomarkers of hepatocellular carcinoma exhibited a lower EC50 as compared with the positive control, i.e. a constitutively HSVtk expressing vector (FIG. 3).

### Antisense Safety Domains Blocking the Splice Site of the Trans-Splicing RNA do not Impair On-Target Trans-Splicing but Suppress Off-Target Trans-Splicing

Another embodiment of this invention are novel safety domains, i.e., antisense sequences within the trans-splicing RNAs that are complementary to, at least a part of its splice signal or domain, or its splice donor (SD) and polypyrimidine tract (PPY). These safety domains prevent off-target trans-splicing as any of the target binding domains has to bind to its target first to release the safety domain from the SD and PPY in order to enable on-target trans-splicing. Two designs of safety domains were invented and tested: 1. A continuous safety domain and 2. a segmented safety domain the latter of which was disrupted by the various target binding domains (FIG. 4). The safety domains did not impair the suicide activity of the trans-splicing-based suicide RNAs (FIG. 4). To monitor off-target trans-splicing, a GFP reporter assay was developed (FIG. 5). This assay did not indicate any off-target activity triggered by trans-splicing suicide RNAs featured with safety domains (FIG. 6).

### Dual Targeting Trans-Splicing Based Suicide RNAs Targeting HBV-Derived and HCC-Linked Pre-mRNA Targets Efficiently Kill HBV-RNA Positive Hepatoblastoma-Derived Human Tissue Culture Cells

Another embodiment of this invention are trans-splicing-based suicide RNAs targeting HBV-positive cells. These suicide RNAs are featured with target binding domains which are complementary to the HBV pre-genomic RNA and AFP or GPC3. Though the expression of the HBV pre-genomic RNA is low in tissue culture cells, the HBV targeting suicide RNAs triggered a comparable cell death activity as the dual HCC targeting suicide RNAs (FIG. 7). HBV derived RNA targets are expressed at much higher levels in HBV-infected cells in vivo.

### Trans-Splicing-Based Suicide RNAs Targeting Pre-mRNAs of Common Cancer Biomarkers Effectively Kill Nasopharyngeal Cancer Cells

Another embodiment of this invention are trans-splicing-based suicide RNAs effectively killing nasopharyngeal cancer cells (FIG. 8). These suicide RNAs are featured with target binding domains which are complementary to various oncogenic pre-mRNAs including the alpha-feto protein (AFP), Vascular endothelium growth factor (VEGF), γ-glutamyl transferase (GGT), Hepatocellular carcinoma associated protein 2 (HCCA2), Transforming growth factor beta 1 (TGF-β1), cluster of differentiation 24 (CD24), Cyclin D1 (CCND1), Glypican 3 (GPC3) and Telomerase reverse transcriptase (TERT) pre-mRNAs.

Another embodiment of this invention are trans-splicing-based suicide RNAs targeting EBV-positive cells harbouring target binding domains which are complementary to Epstein-Barr virus pre-mRNAs (i.e., BZLF1, EBNA-3B, LMP1 and LMP2A).

Dumbbell Vectors Expressing Trans-Splicing RNA can Efficiently be Delivered into Various Cell Types of the Epidermis Following Topical Non-Invasive Application Triggering Cell Type Specific GFP Expression or Cell Death

Another embodiment of this invention are trans-splicing RNAs and dumbbell-shaped delivery vectors targeting epidermal cells harbouring target binding domains which are complementary to Keratin 1 (KRT1), Keratin 2 (KRT2), Keratin 10 (KRT10), Keratin 14 (KRT14), Caspase-14 precursor (CASP14), Neuroblast differentiation-associated protein 2 (AHNAK2). Another embodiment of this invention are trans-splicing RNAs targeting basal cells harbouring target binding domains which are complementary to Keratin 15 (KRT15), Collagen 17A1 (COL17A1), Tumour protein 73 (TP73). Another embodiment of this invention are trans-splicing RNAs targeting hair follicle cells harbouring target binding domains which are complementary to Homeobox C13 (HOXC13), Fibroblast growth factor 7 (FGF-7). Another embodiment of this invention are trans-splicing RNAs targeting senescent cells harbouring target binding domains which are complementary to Forkhead Box 04 (FOXO4) and cyclin-dependent kinase inhibitor 2A (p16). All these trans-splicing RNAs were either featured with a GFP gene for imaging or the HSVtk gene to trigger cell death (FIG. 9). The sequences were delivered using dumbbell vectors into the epidermis of domestic pigs following non-invasive topical application.

### Apoptosis or Necrosis Inducing Proteins can Replace the HSVtk/GCV System for Suicide Gene Therapy

Another embodiment of this invention are trans-splicing-based suicide RNAs encoding other death signals than the HSVtk such as CYLD Lysine 63 Deubiquitinase (CYLD), tumor necrosis factor-like weak inducer of apoptosis (TWEAK), Tumor necrosis factor-related apoptosis-inducing ligand (TRAIL) and Tumor necrosis factor alpha (TNF-α) pre-mRNA. Transfection of HEK293T cells with plasmids expressing these cell death proteins triggered cell death relative to the controls (FIG. 10).

Increasing the Size of One Dumbbell Vector Loop does not Impair Gene Expression

Dumbbell-shaped DNA vectors are unique in the way how they combine double-stranded expression cassettes with single-stranded loops. Another embodiment of this invention are dumbbell vectors with residues for targeted delivery non-covalently attached to the loops via complementary base pairing (FIG. 11). The targeting residues were covalently linked to antisense oligonucleotides (DNA or RNA) with complementarity to one of the dumbbell loops to which we refer as conjugation-loop in the following. To facilitate conjugation, the conjugation-loop size was enlarged from 4 nt, the standard loop size, to 41 or 71 nt using sequences that were predicted to fold no or as little as possible intrinsic DNA secondary structure (FIG. 11). HEK293T cells were then transfected with MaxGFP-expressing dumbbells featured with a 4 nt, 41 nt or 71 nt conjugation-loops and MaxGFP expression was quantified (FIG. 12). The increasing conjugation-loop size did not impair gene expression indicating that larger loops do not compromise nuclear vector diffusion. Notably, dumbbell vectors produced using gap-primer PCR or a combination of gap-primer PCR and the enzymatic ligation assisted by nucleases (ELAN) method harbouring mismatches in one or both terminal stem loop structures, triggered significantly higher levels of gene expression as compared with perfectly base-paired dumbbells and at a level that was comparable with the expression triggered by a plasmid. As larger conjugation-loops are expected to facilitate binding of the antisense DNA or RNA oligonucleotides and formation of a resulting B- or H-form helix, we proceeded with 57 nt conjugation-loops for residue conjugation.

Non-Covalent Conjugation of Dumbbell Vector DNA with GalNAc3 and aptCD137-2 Residues Via Complementary Base Pairing

The residues that were attached were tri-antennary GalNAc residues (GalNAc3) or homo-dimers of CD137-binding aptamers (aptCD137-2). The GalNAc3 residues were attached to the 3′ ends of rather unstructured DNA or RNA oligonucleotides (FIG. 13). AptCD137 was attached 5′ and 3′ towards an RNA oligonucleotide. The conjugation-loops were designed to be completely unstructured or to exhibit as little as possible internal secondary structure formation and to harbour a central 21 nt antisense oligonucleotide binding domain with two adjunct 18 nt spacers, one on each side (FIG. 13A). To non-covalently attach the GalNAc3 and aptCD137-2 residues to the dumbbells' conjugation loops, the residues were covalently linked to loop binding antisense oligonucleotides (FIG. 13B). GalNAc3 labelled DNA and RNA oligonucleotides were generated by chemical synthesis. The two aptamer domains of the aptCD137-2 homodimer were bridged via a RNA linker and the aptCD137-2 sequence was generated using in vitro transcription. The GalNAc3 and aptCD137-2 labelled oligonucleotides were then annealed to the conjugation-loops of HSVtk and/or MaxGFP-expressing dumbbell vectors (FIG. 13B). The molecular weights of the MaxGFP- and HSVtk-expressing dumbbells were both with 1.3×10⁶ g/mol relatively large compared with the molecular weight of the GalNAc3 labelled oligonucleotides, exhibiting molecular weights of 8.4×10³. To monitor the successful conjugation using electrophoretic mobility shift assays, the dumbbell-GalNAc3—but not the dumbbell-aptCD137-2-conjugates were cleaved using the AseI restriction endonuclease before analysing the resulting fragments on agarose gels (FIG. 13 C,D). The use of stoichiometric oligonucleotide to dumbbell ratios for annealing resulted in incomplete GalNAc3 conjugation of the dumbbell vector DNA; however, virtually 100% conjugation efficiency was achieved when the RNA- or DNA-GalNAc3 oligonucleotides were used in 2- or 10-fold molar excess (FIG. 13C). In case of the aptCD137-2 aptamer conjugation, an equal molar amount or a 2-fold molar excess of the aptamer yielded a conjugation efficiency of 80% or 81%, respectively (FIG. 13D).

GalNAc3-RNA but not-DNA Linkers are Cleavable by RNaseH

RNA in heteroduplexes formed between complementary RNA and DNA can be cleaved by endogenous RNaseH. In the consequence, dumbbell RNA but not DNA linker-conjugates can release the GalNAc3 residue form the dumbbell after delivery into the cytoplasm to facilitate diffusion of the dumbbell through the nuclear pore complex. We investigated the cleavability of our dumbbell-GalNAc3 conjugates by RNaseH using an in vitro assay. Therefore, the conjugates were exposed to RNaseH for 120 min. To better visualise release of the small GalNAc3 residue from the relatively large dumbbell vector DNA using a gel shift assay, the conjugation loops were cleaved off with AseI before loading the samples on a gel. As expected, the gel shift assay indicates cleavability and release of the GalNAc3 residue form the RNA—but not the DNA-linker dumbbell conjugate (FIG. 13E).

### Dumbbell-GalNAc3 Conjugates are Taken Up by Hepatoblastoma-Derived Human Tissue Culture Cells Triggering MaxGFP Expression

MaxGFP expressing dumbbell vectors were manufactured and proven to be exonuclease resistant (FIG. 15). MaxGFP Dumbbell-GalNAc3 conjugates were generated (FIG. 14 and FIG. 15) and mixed with trypsinized and pelleted HepG2 cells for 4 hours before seeding the cells and after 48 hours we quantified the levels of dumbbell vector DNA uptake using qPCR (FIGS. 14 and 15B). Our data show that dumbbell-DNA- and dumbbell-RNA-GalNAc3 conjugates were taken up by HepG2 cells with comparable efficiency (FIG. 15B). However, uptake of dumbbell-GalNAc3 conjugates from the medium was significantly less efficient compared with delivery via lipofection. As dumbbell DNA might be adsorbed at the cell surface or stay unproductive in endosomes, we also quantified the transcribed MaxGFP mRNA using RT-qPCR (FIG. 15C). Slightly more maxGFP mRNA was detected for the dumbbell-RNA- as compared with the dumbbell-DNA conjugates, but the difference was not significant. The signals indicating the presence of db vector DNA or maxGFP mRNA in the NTCs are presumably a background signal which is either due to presence of a minor contamination of the RT-qPCR or an unspecific signal originating from the SYBR Green-based quantification protocol. To further improve the uptake and subsequent expression of dumbbell-GalNAc3 conjugates, two GalNAc3 residues were conjugated via an extended 71 nt conjugation loop harbouring two antisense oligonucleotide binding sites (FIG. 15D). Single and double GalNAc3 dumbbell conjugates linked via RNA- and DNA-linkers, were then incubated with HepG2 cells as described above and after 48 hours we quantified the levels of MaxGFP protein expression using flow cytometry (FIG. 16). On average, dumbbell-RNA-GalNAc3 conjugates gave more MaxGFP-positive cells (49.8%) compared with dumbbell-DNA-GalNAc3 conjugates (28.8%) (FIG. 15E). No difference of MaxGFP expression was observed comparing dumbbell single and double GalNAc conjugates.

HSVtk Expressing Dumbbells Featured with Two GalNAc3 Residues at One Conjugation Loop Triggered Death of Hepatoblastoma-Derived Human Tissue Culture Cells Upon Ganciclovir Treatment

To develop dumbbell-GalNAc conjugates for suicide gene therapy of hepatocellular carcinoma (HCC), two GalNAc3 residues were attached via RNA linker oligonucleotides to a HSVtk expressing dumbbell vector (FIG. 17A). To test the cell death activity of the conjugates, the vectors were added to the culture medium of HepG2 cells. The HSVtk dumbbell-2-GalNAc3 double-conjugate but not the HSVtk dumbbell-1-GalNAc3 conjugate featured with only one GalNAc3 residue, triggered significant death of HepG2 cells, i.e. a 34.7% reduction of cell viability at day 6 in an alamarBlue cell viability assay, upon 100 μM GCV treatment (FIG. 17B). For comparison, lipofection of HepG2 cells with an HSVtk expressing plasmid or double-conjugated dumbbell vector reduced the cell viability by 49.8% or 54.2%. These data also indicate that dumbbell conjugation with 2 GalNAc3 residues via an RNA linker did not impair gene expression as compared with the unconjugated dumbbell. Notably, no significant reduction of cell viability was observed in the absence of GCV treatment.

Dumbbell-shaped DNA vectors increasingly raise attention as a promising versatile naked DNA based delivery vector system for gene therapeutic applications and for genetic vaccination. As opposed to plasmids and DNA minicircles, dumbbells can be covalently or non-covalently conjugated with helper functions for imaging, immune sensing or targeted delivery via the single-stranded loops. The are latter formed by chemically synthesized oligodeoxyribonucleotides which may be chemically modified, and which can either be added by direct ligation or modelled from primers used in a PCR reaction. Loop conjugation of helper functions is not expected to impair the transcriptional activity of dumbbell vectors but may affect cellular and nuclear targeting. We investigated non-covalent linkage of GalNAc3 and aptCD137-2 residues for targeted delivery into hepatocytes and nasopharyngeal cancer cells. Therefore, antisense oligonucleotides (DNA or RNA) were covalently attached to these residues which could then be annealed via complementary base pairing towards extended conjugation loops. Extension of the conjugation loops, though in the absence of residue conjugation, did not impair dumbbell vector-based gene expression (FIG. 12), and it is reasonable to assume that it did not impair nuclear diffusion either. That might be explained by the design of the loops which were selected to be uncapable of forming internal secondary structures which would render the dumbbells more bulky and more difficult to diffuse through the nuclear pore complex. Both RNA and DNA linker oligonucleotides could successfully attach residues to the conjugation loop of the dumbbell. We observed some evidence that conjugation via RNA linkers was more efficient than conjugation via DNA linkers as a lower excess of the oligo over the dumbbell yielded more conjugates. This finding may be explained by the higher stability of RNA: DNA base pairs as compared with DNA: DNA base pairs which would facilitate the nucleation process provided the number of RNA nucleation sites is not reduced due to secondary structure formation. A reduction of nucleation sites can be excluded in our example as both, the RNA and DNA conjugation oligonucleotides, were selected to be rather unstructured (FIG. 11). In addition, the RNA but not the DNA linker could be cleaved by RNaseH to decouple the GalNAc3 residue and the dumbbell vector (FIG. 13E). The use of cleavable or stimuli-labile linkers is strongly advised if large residues are being attached to a genetic vector or effector molecule. In our example, the GalNAc3 residue was rather small compared with the size of the dumbbell vector. Nevertheless, the use of an RNaseH-cleavable RNA linker did increase the number of MaxGFP-positive cells on average indicating release of the dumbbell from the GalNAc3 residue and more efficient nuclear targeting of the unconjugated dumbbell DNA (FIG. 18). While siRNA-GalNAc3 conjugates represent the clinical standard for targeted delivery of siRNA into hepatocytes, conjugation of GalNAc3 residues or aptamers to gene expression vectors including dumbbell vectors for targeted delivery has not yet been reported. In this study, we demonstrated that conjugation of GalNAc3 residues to a 2186 bp MaxGFP-expressing dumbbell vector can facilitate vector delivery into HepG2 cells resulting in 29 to 51% MaxGFP-positive cells as measured using flow cytometry analyses (FIG. 15E). According to our calculations which are based on the cellular uptake of single GalNAc3 conjugates, about 10 dumbbell-GalNAc3 conjugate complexes were delivered in these experiments on average per cell. In addition, a HSVtk-expressing dumbbell featured with two GalNAc3 residues at a single conjugation loop triggered 34.7% death of HepG2 after addition to the cell culture medium in the presence of 100 μM GCV. The equivalent dumbbell-conjugate featured with only one GalNac3 residue did not exhibit a significant effect. The observation that double GalNAc3 conjugates triggered more cell death compared with single GalNAc3 conjugates indicates that multiple GalNAc3 residues attached to a single dumbbell may facilitate binding towards multiple ASGPR receptors and subsequent cellular uptake (FIG. 18). However, this effect was not observed with MaxGFP expressing dumbbells and requires further investigation.

These data indicate that GalNac3-mediated targeted delivery of gene expression vectors such as dumbbells works, and it works better if more than one GalNac3 residue is conjugated. In principle, dumbbell vectors can be conjugated with more than two GalNac3 residues per conjugation loop and both dumbbell loops can be explored as conjugation loops to further improve delivery into hepatocytes. Though the MaxGFP expression triggered by cellular uptake of dumbbell-GalNAc3 conjugates was readily detectable using flow cytometry, it was scarcely visible under the fluorescence microscope with only single cells showing bright fluorescence. On the other hand, uptake of HSVtk dumbbells effectively killed the targeted cells. This observation that HSVtk-expressing suicide vectors trigger a stronger phenotype than MaxGFP expression vectors may be explained by any or both of the following reasons: 1. A smaller dumbbell DNA cargo load which may be sufficient to kill a cell might not yet efficiently stain it with MaxGFP for detection, or 2. cells which were not primarily targeted by the dumbbell-conjugates might have been killed by the bystander effect that has been reported for the HSVtk/GCV gene-directed enzyme prodrug system. Notably, HepG2 cells express significantly less ASGPR on their surface as compared with primary hepatocytes. In the consequence, one would expect stronger uptake of dumbbell-GalNAc3-conjugates by primary hepatocytes ex vivo or in vivo. In summary, we demonstrated that dumbbell vectors can efficiently be conjugated with helper functions for targeted delivery via cleavable linkers. Our liver cancer-targeting GalNac3-conjugated suicide vectors are currently being tested in patient-derived xenograft (PDX) nude and humanised mouse models of HCC. As opposed to LNPs which can also be conjugated with helper functions including GalNAc3, naked dumbbell-conjugates are significantly smaller and expected to exhibit facilitated diffusion rates in the extracellular matrix. In the consequence, dumbbell-conjugates may identify single cells including cancer cells or metastasis more efficiently providing a minimalistic vector system that can complement or replace existing viral and non-viral carriers for gene therapeutic applications.

Dumbbell-GalNAc3 Conjugates Trigger Strong mRNA Expression in Murine Livers

Mice were injected iv with GFP expressing naked dumbbells (hydrodynamic injection), dumbbell-LNPs or dumbbell-GaNAc3 conjugates. Dumbbell-GalNAc3 conjugates were found to trigger highest levels of GFP mRNA expression in murine livers as quantified by RT-qPCR (FIG. 19).

### REFERENCES

Eul, J. (2006). Method for the repair of mutated RNA from genetically defective DNA and for the specific destruction of tumor cells by RNA trans-splicing and a method for the detection of naturally trans-spliced cellular RNA, United States Patent Application, US20060094675A1.
Fairbrother, W. G., Yeh, R. F., Sharp, P. A., and Burge, C. B. (2002). Predictive identification of exonic splicing enhancers in human genes. Science 297, 1007-1013.
Gibbs, P. E., Zielinski, R., Boyd, C., and Dugaiczyk, A. (1987). Structure, polymorphism, and novel repeated DNA elements revealed by a complete sequence of the human alpha-fetoprotein gene. Biochemistry 26, 1332-1343.
Johansson, C., and Schwartz, S. (2013). Regulation of human papillomavirus gene expression by splicing and polyadenylation. Nat Rev Microbiol 11, 239-251.
Kozak, M. (1995). Adherence to the first-AUG rule when a second AUG codon follows closely upon the first. Proc Natl Acad Sci USA 92, 2662-2666.
Kozak, M. (1999). Initiation of translation in prokaryotes and eukaryotes. Gene 234, 187-208.
Mccarthy, E. M., and Phillips, J. A., 3rd. (1998). Characterization of an intron splice enhancer that regulates alternative splicing of human GH pre-mRNA. Hum Mol Genet 7, 1491-1496.
Poddar S, Loh P S, Ooi Z H, Osman F, Eul J, Patzel V. RNA structure design improves activity and specificity of trans-splicing triggered cell death in a suicide gene therapy approach. Molecular Therapy: Nucleic Acids 11, 41-56, 2018.
Reese, M. G., Eeckman, F. H., Kulp, D., and Haussler, D. (1997). Improved splice site detection in Genie. J Comput Biol 4, 311-323.
Saksmerprome, V., Roychowdhury-Saha, M., Jayasena, S., Khvorova, A., and Burke, D. H. (2004). Artificial tertiary motifs stabilize trans-cleaving hammerhead ribozymes under conditions of submillimolar divalent ions and high temperatures. RNA 10, 1916-1924.
Schmitt, M., and Pawlita, M. (2011). The HPV transcriptome in HPV16 positive cell lines. Mol Cell Probes 25, 108-113.
Wang, M., and Marin, A. (2006). Characterization and prediction of alternative splice sites. Gene 366, 219-227.

1. A trans-splicing RNA (tsRNA) molecule comprising:
(a) at least one binding domain specific for at least a part of a gene that associates with or is a biomarker for a cell or a disease to be treated; and
(b) nucleic acids encoding at least one or more expressible
(c) suicide protein or a protein that is a component of a suicide system; or
(d) fluorescent protein, luciferase or other reporter protein; or
(e) therapeutic protein; and
(f) at least one splice signal; and
(g) at least one safety domain specific for the splice signal or splice site within the trans-splicing RNA.

2. The trans-splicing RNA (tsRNA) molecule of claim 1 wherein: said binding domain comprises a binding site comprising at least 25, 35, 45, 55 or more consecutive unstructured nucleotides (nt) having no internal binding and/or self-complementary sequences and; said binding domain, when of a length of 44 nt or longer, has at least one, or a plurality of, mismatch nucleotide(s) with respect to said gene and/or
wherein: said safety domain is an antisense binding domain specific for a splice site in the splice signal whereby the safety domain prevents off-target trans-splicing and/or
wherein said safety domain is either a linear sequence of nucleic acids, termed a continuous safety domain, or a folded sequence of nucleic acids, termed a segmented safety domain, comprising one or more folds; and/or
wherein said tsRNA further comprises:
at least one binding domain specific for at least a part of a gene that is ubiquitously expressed in any cell and/or
wherein said biomarker is any single biomarker or combination of biomarkers selected from the following list: a cancer marker, HCC biomarkers alpha-feto protein (AFP), Vascular endothelium growth factor (VEGF), □-glutamyl transferase (GGT), Hepatocellular carcinoma associated protein 2 (HCCA2), Transforming growth factor beta 1 (TGF-β1), cluster of differentiation 24 (CD24), Cyclin D1 (CCND1), Glypican 3 (GPC3), Telomerase reverse transcriptase (TERT), α-L-fucosidase (AFU), CD19, CD34, CD44, CD49E, CD51, CD105, Collagen type XV alpha 1 (COL15A1), C-X-C motif chemokine receptor 4 (CXCR4), Denticleless E3 ubiquitin protein ligase homolog (DTL), Epithelial cell adhesion molecule (EPCAM), Golgi protein 73 (GP73), G protein signaling modulator 2 (GPSM2), Hepatocyte growth factor (HGF), Heat shock protein 70 (HSP70), Insulin like growth factor 2 (IGF2), Immunoglobulin superfamily member 3 precursor (IGSF3), Integrin Subunit Alpha 6 (ITGA6), Kell blood group glycoprotein (KEL), KIT Proto-Oncogene, Receptor Tyrosine Kinase (KIT), Minichromosome Maintenance Complex Component 3 (MCM3), Minichromosome Maintenance Complex Component 7 (MCM7), PDZ Binding Kinase (PBK), DNA Polymerase Delta 1, Catalytic Subunit (POLD1), Protein Regulator Of Cytokinesis 1 (PRC1), SRY-Box Transcription Factor 17 (SOX17), Spermatogenesis-associated serine-rich protein 2 (SPATS2), Translocon-associated protein subunit beta (SSR2), Stathmin 1 (STMN1), Thrombomodulin (THBD), ZW10 Interacting Kinetochore Protein (ZWINT), HBV-derived RNA including HBV pgRNA, Epstein-Barr virus derived RNA and pre-mRNAs including BamHI Z Epstein-Barr virus replication activator (BZLF1), Epstein-Barr virus nuclear antigen 3B (EBNA-3B), Latent membrane protein 1 (LMP1), and Latent membrane protein 2A (LMP2A), epidermal cell markers including Keratin 1 (KRT1), Keratin 2 (KRT2), Keratin 10 (KRT10), Keratin 14 (KRT14), Caspase-14 precursor (CASP14), Neuroblast differentiation-associated protein 2 (AHNAK2), basal cell markers including Keratin 15 (KRT15), Collagen 17A1 (COL17A1), Tumour protein 73 (TP73), hair follicle cell markers including Homeobox C13 (HOXC13) and Fibroblast growth factor 7 (FGF-7), senescent cell markers including Forkhead Box 04 (FOX04) and cyclin-dependent kinase inhibitor 2A (p16), the stratum corneum markers Kallikrein related peptidase 5 (KLK5), Small proline-rich protein 4 (SPRR4), and Arachidonate 12-lipoxygenase (ALOX12B), the Stratum spinosum (Upper epidermal layers) markers HOP homeobox (HOPX) and Kallikrein 9 (KLK9), the Stratum granulosum markers Filaggrin (FLG) and Premature ovarian failure 1B protein (POF1B), the Melanocyte markers Melan-A (MLANA) and Tyrosinase (TYR), the Langerhans cell markers CD1A and CD207, the fibroblast markers Periostin (POSTN) and Phospholipase C-eta-2 protein (PLCH2), the basal cell carcinaoma markers Glioma 1 (GII1), Glioma 2 (G112), Forkhead box protein (FOXM1), Forkhead box protein (FOXO3A), Desmoglein 2 (DSG2) and C3b, the basal cell carcinoma recurrence markers Cyclooxygenase (COX-2), Ezrin (EZR), CD25, Maspin, Glioma 3 (GlI3), GalNAc3 and Gremlin1 and/or
wherein said at least one or more suicide protein or at least one or more protein that is a component of a suicide system is selected from the group comprising or consisting of: HSVtk, CYLD Lysine 63 Deubiquitinase (CYLD), tumor necrosis factor-like weak inducer of apoptosis (TWEAK), Tumor necrosis factor-related apoptosis-inducing ligand (TRAIL) and Tumor necrosis factor alpha (TNF-α)

3. (canceled)

4. (canceled)

5. (canceled)

6. (canceled)

7. (canceled)

8. The trans-splicing RNA molecule of claim 1, wherein said disease is cancer or a viral infection or a bacterial infection or a genetic disease.

9. The trans-splicing RNA molecule of claim 8 wherein said cancer is selected from the group comprising or consisting of: hepatocellular carcinoma (HCC), cervical cancer, vaginal cancer, vulvar cancer, penile cancer, skin cancers, melanoma including malignant melanoma, squamous-cell carcinoma, basal-cell carcinoma, Merkel cell carcinoma, lung cancer, cell bladder cancer, breast cancer, colon or rectal cancer, anal cancer, endometrial cancer, kidney cancer, leukemia, acute myelogenous or myeloid leukemia (AML), acute lymphoblastic leukemia (ALL), chronic lymphotic leukemia (CML), chronic myelogenous or myeloid leukemia (CML), hairy cell leukemia (HCL), T-cell prolymphocytic leukemia (P-TLL), large granular lymphocytic leukemia, adult T-cell leukemia, lymphoma, myeloma, non-Hodgkin lymphoma, pancreatic cancer, prostate cancer, thyroid cancer, nasopharyngeal cancer, mouth or throat cancer, oropharyngeal cancer, nasopharyngeal cancer, stomach cancer, brain tumours, bone cancer, and stem cell cancer; or
wherein said viral infection is selected from the group comprising or consisting of: an infection with a retrovirus including the human T-cell lymphotropic virus (HTLV) lentiviruses, human immunodeficiency virus types 1 and 2 (HIV-1 and HIV-2), human papillomavirus including types 16 and 18 (HPV-16 and HPV-18), a hepadnavirus including HAV, HBV, HCV, HDV, and HEV, a herpesvirus including herpes simplex (HSV), Epstein-Barr virus (EBV), cytomegalovirus (CMV), an adenovirus, an adeno-associated virus, an influenza virus or an integrating virus and a nasopharyngeal virus; or
wherein said bacterial infection is selected from the group comprising or consisting of: an infection caused by Bartonella henselae, Francisella tularensis, Listeria monocytogenes, salmonella species, Salmonella typhi, Brucella species, Legionella species. Mycobacteria species, Mycobacterium tunberculosis, Nocardia species, Rhodococcus species, Yersinia species and Neisseria meningitides; or
wherein said acquired genetic disease is selected from the group comprising or consisting of: Neurofibromatosis 1 and 2, Mc Cune Albright, Duchenne muscular dystrophy (DMD), Epidermolysis bullosa, Fanconi A and C, Philadelphia chromosome, Hemophilia A and B, cystic fibrosis, Muckle Wells syndrome, lipoprotein lipase deficiency, B-thalassemia, Gaucher Disease types I to III—GBA gene, Ornithine transcarbamylase (OTC) deficiency—OTC, Phenylketonuria (PKU)—PAH gene, Aspartylglucosaminuria—AGA gene, Alpha-1 anti trypsin deficiency (AATD)—SERPINA1, and pyruvate dehydrogenase complex deficiency.

10. (canceled)

11. (canceled)

12. (canceled)

13. The trans-splicing RNA molecule of claim 1, wherein said tsRNA is targeted against nasopharyngeal cells or epidermal cells, basal cells, senescent cells or hair follicles and/or wherein targeting residues are non-covalently linked, via antisense oligonucleotides (DNA or RNA) complementarity to at least a part of the tsRNA molecule; and/or
wherein targeting residues are covalently linked to at least a part of the tsRNA molecule; and/or
wherein at least one tri-antennary GalNAc residues (GalNAc3) or at least one homodimer of CD137-binding aptamer (aptCD137-2) residue is attached to said tsRNA; optionally
wherein at least two GalNAc3 residues are attached to said tsRNA.

14. (canceled)

15. (canceled)

16. (canceled)

17.

18. The trans-splicing RNA molecule of claim 1, wherein at least a part thereof comprises unstructured RNA, having as little as possible internal secondary structure formation, comprising a 21 nt antisense oligonucleotide binding domain with at least one spacer on at least one side; optionally
wherein an 18 nt spacers is provided either side of said binding domain.

19. (canceled)

20. The trans-splicing RNA molecule of claim 1, wherein said tsRNA is either 5′ or 3′ tsRNA; and/or
wherein said trans-splicing RNA is dumbbell shaped; and/or
wherein said trans-splicing RNA comprises at least one residue or helper function for targeted delivery covalently or non-covalently attached to said ts-RNA. at least one of the loops; optionally
wherein said residue or helper function is selected from the group comprising: a carbohydrate, a (GalNAc)3 residue, a nucleic acid, a RNA or DNA or peptide aptamer, a CD137 or a PSMA binding RNA aptamer, a protein, a peptide, a cell penetrating peptide, an antibody, or a CD137-binding antibody

21. (canceled)

22. (canceled)

23. (canceled)

24. A cell containing said ts RNA of claim 1.

25. A vector containing said tsRNA of claim 1; optionally
wherein said vector is a naked nucleic acid based vector, a non-viral vectors, or a viral vector; optionally
wherein said naked nucleic acid based vector is selected from the group comprising comprising or consisting of: a RNA molecule, a plasmid, a DNA minicircle and a dumbbell-shaped DNA vector; or
wherein said non-viral vector is selected from the group comprising or consisting of: a liposomal vesicle, a nanoparticle, a polymer conjugate, an antibody conjugate, a cell penetrating peptide and a polymer capsule; or
wherein said viral vector is selected from the group comprising or consisting of: a retroviral vector, a lentiviral vector, an adenoviral vector, and adeno-associated viral vector, a Herpes simplex viral vector, a vaccinia viral vector, chimeric viral vectors, a sindbis-viral vector, or an alphaviral vector, semliki forest viral vector, and a Venezuelan equine encephalitis viral vector.

26. (canceled)

27. (canceled)

28. (canceled)

29. (canceled)

30. A method of targeting a diseased cell or treating a disease or condition comprising administering the tsRNA of claim 1 or a vector containing the tsRNA, of claim 1 by:
a) an in vivo administration route selected from the group comprising or consisting of: topical application; intranasal application; alveolar application; systemic application; oral application; intravenous application; intramuscular application; subcutaneous application; cutaneous application; intraperitoneal application; or injection into a tumor, or
b) an ex vivo administration selected from the group consisting or comprising of: transfection, lipofection, transduction, electroporation, nucleofection or transformation, optionally, exposing said cell to at least one other component(s) of a suicide system effective to kill said cell.

31. (canceled)

32. (canceled)

33. The method of claim 30, wherein said component(s) of said suicide system is selected from the group comprising or consisting of: ganciclovir, cytosine deaminase-5-fluorocytosine, cytochrome P450-ifosfamide, cytochrome P450-cyclophosphamide, and nitroreductase-5-[aziridin-1-yl]-2,4-dinitrobenzamide.

34. The trans-splicing RNA molecule of claim 1 or a cell, or a method wherein said cell is mammalian, preferably human,
wherein the cell contains said trans-splicing RNA (tsRNA) molecular; and
wherein the method targets a diseased cell or treats and disease or condition, and comprises administering the tsRNA or a vector containing the tsRNA by:
a) an in vivo administration route selected from the group comprising or consisting of: topical application; intranasal application; alveolar application; systemic application; oral application; intravenous application; intramuscular application; subcutaneous application; cutaneous application; intraperitoneal application; or injection into a tumor, or
b) an ex vivo administration selected from the group consisting or comprising of: transfection, lipofection, transduction, electroporation, nucleofection or transformation, optionally, exposing said cell to at least one other component(s) of a suicide system effective to kill said cell.

35. (canceled)

36. A medicament comprising said tsRNA of claim 1 or a vector and, optionally, at least one further component of a suicide system effective to trigger death of a cell expressing said trans-spliced RNA, wherein the vector contains tsRNA; and optionally
wherein said vector is a naked nucleic acid based vector, a non-viral vectors, or a viral vector; optionally
wherein said naked nucleic acid based vector is selected from the group comprising comprising or consisting of: a RNA molecule, a plasmid, a DNA minicircle and a dumbbell-shaped DNA vector; or
wherein said non-viral vector is selected from the group comprising or consisting of: a liposomal vesicle, a nanoparticle, a polymer conjugate, an antibody conjugate, a cell penetrating peptide and a polymer capsule; or
wherein said viral vector is selected from the group comprising or consisting of: a retroviral vector, a lentiviral vector, an adenoviral vector, and adeno-associated viral vector, a Herpes simplex viral vector, a vaccinia viral vector, chimeric viral vectors, a sindbis-viral vector, or an alphaviral vector, semliki forest viral vector, and a Venezuelan equine encephalitis viral vector.

37. A pharmaceutical composition comprising said tsRNA of claim 1 or a vector and, optionally, at least one further component of a suicide system effective to trigger death of a cell expressing said trans-spliced RNA; and a carrier suitable for human or veterinary use; optionally
wherein said one further component of said suicide system is selected from the group comprising or consisting of: ganciclovir, cytosine deaminase-5-fluorocytosine, cytochrome P450-ifosfamide, cytochrome P450-cyclophosphamide, and nitroreductase-5-[aziridin-1-yl]-2,4-dinitrobenzamide; and
wherein the vector is a naked nucleic acid based vector, a non-viral vectors, or a viral vector; optionally
wherein said naked nucleic acid based vector is selected from the group comprising comprising or consisting of: a RNA molecule, a plasmid, a DNA minicircle and a dumbbell-shaped DNA vector; or
wherein said non-viral vector is selected from the group comprising or consisting of: a liposomal vesicle, a nanoparticle, a polymer conjugate, an antibody conjugate, a cell penetrating peptide and a polymer capsule; or
wherein said viral vector is selected from the group comprising or consisting of: a retroviral vector, a lentiviral vector, an adenoviral vector, and adeno-associated viral vector, a Herpes simplex viral vector, a vaccinia viral vector, chimeric viral vectors, a sindbis-viral vector, or an alphaviral vector, semliki forest viral vector, and a Venezuelan equine encephalitis viral vector.

38. (canceled)

39. (canceled)

40. A dumbbell-shaped DNA expression vector comprising:
a) one or more linear or hairpin-shaped transcription cassettes each comprising a nucleotide sequence encoding a nucleic acid molecule to be expressed;
b) two single-stranded DNA loops;
c) operably linked to said transcription cassette a minimal transcription promoter nucleotide sequence and a transcriptional terminator;
d) a nucleotide sequence comprising a DNA sequence that functions as nuclear targeting sequence (NTS);
e) a nucleotide sequence comprising a spliceable intron; and
f) at least one residue or helper function for targeted delivery covalently or non-covalently linked to at least one of the loops; optionally
wherein said residue or helper function is selected from the group comprising: a carbohydrate, a (GalNAc)₃ residue, a nucleic acid, a RNA or DNA or peptide aptamer, a CD137 or a PSMA binding RNA aptamer, a protein, a peptide, a cell penetrating peptide, an antibody, or a CD137-binding antibody; and/or
wherein said helper function is non-covalently attached to one or both of the dumbbells loops via complementary antisense oligonucleotides (RNA or DNA) to which the helper function is covalently attached at the 5′ or 3′ end; and/or
wherein said helper function is covalently attached to one or both of the dumbbells loops; and/or
wherein said NTS comprises a binding site for a transcription factor; and/or
wherein said NTS is the SV40 enhancer sequence, the minimal SV40 enhancer sequence, the smooth muscle cell y-actin (SMGA) promoter or the oriP of the Epstein-Barr virus (EBV).

41. (canceled)

42. (canceled)

43. (canceled)

44. (canceled)

45. (canceled)

46. A pharmaceutical composition comprising said dumbbell-shaped DNA expression vector of claim 40 and, optionally, at least one further component of a suicide system effective to trigger death of a cell expressing said dumbbell-shaped DNA vector; and a carrier suitable for human or veterinary use.

47. (canceled)

48. A dumbbell-shaped DNA expression vector comprising or encoding the trans-splicing RNA molecule of claim 1.


SEQUENCE LISTING (250 sequences)

SEQ ID NO: 1 - DNA, 237 bp, synthetic construct
  source 1..237: mol_type = other DNA
cgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagc

SEQ ID NO: 2 - DNA, 21 bp, synthetic construct
  source 1..21: mol_type = other DNA
ctggaaaaaaagggaaaaaaa

SEQ ID NO: 3 - DNA, 50 bp, synthetic construct
  source 1..50: mol_type = other DNA
  misc_feature 15: note = AP-1 binding site between residues 15 and 16 having has a nucleotide sequence of ATGAGTCAT
atccagttttctggatgactagttagttattaatagtaatcaattacggg

SEQ ID NO: 4 - DNA, 42 bp, synthetic construct
  source 1..42: mol_type = other DNA
  misc_feature 15: note = AP-1 binding site between residues 15 and 16 having a nucleotide sequence of ATGAGTCAT
aaggtcttttgacctagatacattgatgagtttggacaaacc

SEQ ID NO: 5 - DNA, 79 bp, synthetic construct
  source 1..79: mol_type = other DNA
ctagcgaccagttttattttattttattttagttctcatgcacacttatagcggtttggtttggtttggtaactggtcg

SEQ ID NO: 6 - DNA, 93 bp, synthetic construct
  source 1..93: mol_type = other DNA
  misc_feature 72: note = AP-1 binding site between residues 72 and 73 having a nucleotide sequence of ATGAGTCAT
atccagttttattttattttattttagttctcatgcacacttatagcggtttggtttggtttggtaactggagcgatgtacgggccagatata

SEQ ID NO: 7 - DNA, 99 bp, synthetic construct
  source 1..99: mol_type = other DNA
  misc_feature 78: note = AP-1 binding site between residues 78 and 79 having a nucleotide sequence of ATGAGTCAT
atccagttttattttagttctcatgcacacttatagggaaacccgttctcatgcacacttatattggtttgtactggagcgatgtacgggccagatata

SEQ ID NO: 8 - DNA, 99 bp, synthetic construct
  source 1..99: mol_type = other DNA
  misc_feature 78: note = AP-1 binding site between residues 78 and 79 having a nucleotide sequence of ATGAGTCAT
atccagttttattttagttctcatgcacacttatagggaaacccgttctcatgcacacttatattggtttgtactggagcttcccaaccttaccagagg

SEQ ID NO: 9 - DNA, 21 bp, synthetic construct
  source 1..21: mol_type = other DNA
gctataagtgtgcatgagaac

SEQ ID NO: 10 - RNA, 21 nt, synthetic construct
  source 1..21: mol_type = other RNA
gctataagtgtgcatgagaac

SEQ ID NO: 11 - RNA, 222 nt, synthetic construct
  source 1..222: mol_type = other RNA
gggagagaggaagagggatgggcgaccgaacgtgcccttcaaagccgttcactaaccagtggcataacccagaggtcgatagtactggatcccccccccgctataagtgtgcatgagaaccccggggggagagaggaagagggatgggcgaccgaacgtgcccttcaaagccgttcactaaccagtggcataacccagaggtcgatagtactggatcccccc

SEQ ID NO: 12 - RNA, 96 nt, synthetic construct
  source 1..96: mol_type = other RNA
gggagagaggaagagggatgggcgaccgaacgtgcccttcaaagccgttcactaaccagtggcataacccagaggtcgatagtactggatcccccc

SEQ ID NO: 13 - RNA, 71 nt, synthetic construct
  source 1..71: mol_type = other RNA
gggaggacgatgcggatcagccatgtttacgtcactccttgtcaatcctcatcggcagacgactcgcccga

SEQ ID NO: 14 - DNA, 233 bp, synthetic construct
  source 1..233: mol_type = other DNA
gctagcgtagcccggcaacagttcctttatacaaattcaccagatgatacaaaacatatttttttagccgggaagcgccgaatcgatcgtgatctaaagatcctctacaccaacaaaattattatgtttattaaactgacgatcgaatggtacctctgaaacaccttccttgactctttcccacgcctccaccaccttcaacctctttcttcgcctcccccatcttcctcagc

SEQ ID NO: 15 - DNA, 50 bp, synthetic construct
  source 1..50: mol_type = other DNA
aacagttcctttatacaaattcaccagatgatacaaaacatattttttta

SEQ ID NO: 16 - DNA, 50 bp, synthetic construct
  source 1..50: mol_type = other DNA
gatctaaagatcctctacaccaacaaaattattatgtttattaaactgac

SEQ ID NO: 17 - DNA, 50 bp, synthetic construct
  source 1..50: mol_type = other DNA
acaccttccttgactctttcccacgcctccaccaccttcaacctctttct

SEQ ID NO: 18 - DNA, 10 bp, synthetic construct
  source 1..10: mol_type = other DNA
gtagcccggc

SEQ ID NO: 19 - DNA, 24 bp, synthetic construct
  source 1..24: mol_type = other DNA
gccgggaagcgccgaatcgatcgt

SEQ ID NO: 20 - DNA, 19 bp, synthetic construct
  source 1..19: mol_type = other DNA
gatcgaatggtacctctga

SEQ ID NO: 21 - DNA, 16 bp, synthetic construct
  source 1..16: mol_type = other DNA
tcgcctcccccatctt

SEQ ID NO: 22 - DNA, 2366 bp, synthetic construct
  source 1..2366: mol_type = other DNA
actagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagcgtagcccggcaacagttcctttatacaaattcaccagatgatacaaaacatatttttttagccgggaagcgccgaatcgatcgtgatctaaagatcctctacaccaacaaaattattatgtttattaaactgacgatcgaatggtacctctgaaacaccttccttgactctttcccacgcctccaccaccttcaacctctttcttcgcctcccccatcttcctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctgcttcgtacccctgccatcaacacgcgtctgcgttcgaccaggcggcgcgatcacggggacacagcaaccgacggacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 23 - DNA, 2603 bp, synthetic construct
  source 1..2603: mol_type = other DNA
cgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcactagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagcgtagcccggcaacagttcctttatacaaattcaccagatgatacaaaacatatttttttagccgggaagcgccgaatcgatcgtgatctaaagatcctctacaccaacaaaattattatgtttattaaactgacgatcgaatggtacctctgaaacaccttccttgactctttcccacgcctccaccaccttcaacctctttcttcgcctcccccatcttcctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctgcttcgtacccctgccatcaacacgcgtctgcgttcgaccaggcggcgcgatcacggggacacagcaaccgacggacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 24 - RNA, 1726 nt, synthetic construct
  source 1..1726: mol_type = other RNA
tcagatcgctagcgtagcccggcaacagttcctttatacaaattcaccagatgatacaaaacatatttttttagccgggaagcgccgaatcgatcgtgatctaaagatcctctacaccaacaaaattattatgtttattaaactgacgatcgaatggtacctctgaaacaccttccttgactctttcccacgcctccaccaccttcaacctctttcttcgcctcccccatcttcctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctgcttcgtacccctgccatcaacacgcgtctgcgttcgaccaggcggcgcgatcacggggacacagcaaccgacggacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgc

SEQ ID NO: 25 - DNA, 1919 bp, synthetic construct
  source 1..1919: mol_type = other DNA
actagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagcgtagcccggcaacagttcctttatacaaattcaccagatgatacaaaacatatttttttagccgggaagcgccgaatcgatcgtgatctaaagatcctctacaccaacaaaattattatgtttattaaactgacgatcgaatggtacctctgaaacaccttccttgactctttcccacgcctccaccaccttcaacctctttcttcgcctcccccatcttcctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 26 - DNA, 2156 bp, synthetic construct
  source 1..2156: mol_type = other DNA
cgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcactagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagcgtagcccggcaacagttcctttatacaaattcaccagatgatacaaaacatatttttttagccgggaagcgccgaatcgatcgtgatctaaagatcctctacaccaacaaaattattatgtttattaaactgacgatcgaatggtacctctgaaacaccttccttgactctttcccacgcctccaccaccttcaacctctttcttcgcctcccccatcttcctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 27 - RNA, 1279 nt, synthetic construct
  source 1..1279: mol_type = other RNA
tcagatcgctagcgtagcccggcaacagttcctttatacaaattcaccagatgatacaaaacatatttttttagccgggaagcgccgaatcgatcgtgatctaaagatcctctacaccaacaaaattattatgtttattaaactgacgatcgaatggtacctctgaaacaccttccttgactctttcccacgcctccaccaccttcaacctctttcttcgcctcccccatcttcctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgc

SEQ ID NO: 28 - DNA, 222 bp, synthetic construct
  source 1..222: mol_type = other DNA
gctagcgtagcccggctagatagatagataggaagaaataagtaagaataacaagcaaacaacaaaagccgggaagcgccgaatcgatcgtgatctaaagatcctctacaccaacaaaattattatgtttattaaactgacgatcgaatggtacctctgatttctctttccattgctctttaacctaatttcctgtgtccaattattcccttccccctcagc

SEQ ID NO: 29 - DNA, 51 bp, synthetic construct
  source 1..51: mol_type = other DNA
tagatagatagataggaagaaataagtaagaataacaagcaaacaacaaaa

SEQ ID NO: 30 - DNA, 50 bp, synthetic construct; identical to SEQ ID NO: 16
  source 1..50: mol_type = other DNA

SEQ ID NO: 31 - DNA, 55 bp, synthetic construct
  source 1..55: mol_type = other DNA
tttctctttccattgctctttaacctaatttcctgtgtccaattattcccttccc

SEQ ID NO: 32 - DNA, 10 bp, synthetic construct
  source 1..10: mol_type = other DNA
gtagcccggc

SEQ ID NO: 33 - DNA, 24 bp, synthetic construct
  source 1..24: mol_type = other DNA
gccgggaagcgccgaatcgatcgt

SEQ ID NO: 34 - DNA, 19 bp, synthetic construct
  source 1..19: mol_type = other DNA
gatcgaatggtacctctga

SEQ ID NO: 35 - DNA, 2355 bp, synthetic construct
  source 1..2355: mol_type = other DNA
actagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagcgtagcccggctagatagatagataggaagaaataagtaagaataacaagcaaacaacaaaagccgggaagcgccgaatcgatcgtgatctaaagatcctctacaccaacaaaattattatgtttattaaactgacgatcgaatggtacctctgatttctctttccattgctctttaacctaatttcctgtgtccaattattcccttccccctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctgcttcgtacccctgccatcaacacgcgtctgcgttcgaccaggcggcgcgatcacggggacacagcaaccgacggacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 36 - DNA, 2592 bp, synthetic construct
  source 1..2592: mol_type = other DNA
cgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcactagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagcgtagcccggctagatagatagataggaagaaataagtaagaataacaagcaaacaacaaaagccgggaagcgccgaatcgatcgtgatctaaagatcctctacaccaacaaaattattatgtttattaaactgacgatcgaatggtacctctgatttctctttccattgctctttaacctaatttcctgtgtccaattattcccttccccctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctgcttcgtacccctgccatcaacacgcgtctgcgttcgaccaggcggcgcgatcacggggacacagcaaccgacggacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 37 - RNA, 1715 nt, synthetic construct
  source 1..1715: mol_type = other RNA
tcagatcgctagcgtagcccggctagatagatagataggaagaaataagtaagaataacaagcaaacaacaaaagccgggaagcgccgaatcgatcgtgatctaaagatcctctacaccaacaaaattattatgtttattaaactgacgatcgaatggtacctctgatttctctttccattgctctttaacctaatttcctgtgtccaattattcccttccccctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctgcttcgtacccctgccatcaacacgcgtctgcgttcgaccaggcggcgcgatcacggggacacagcaaccgacggacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgc

SEQ ID NO: 38 - DNA, 1908 bp, synthetic construct
  source 1..1908: mol_type = other DNA
actagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagcgtagcccggctagatagatagataggaagaaataagtaagaataacaagcaaacaacaaaagccgggaagcgccgaatcgatcgtgatctaaagatcctctacaccaacaaaattattatgtttattaaactgacgatcgaatggtacctctgatttctctttccattgctctttaacctaatttcctgtgtccaattattcccttccccctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 39 - DNA, 2145 bp, synthetic construct
  source 1..2145: mol_type = other DNA
cgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcactagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagcgtagcccggctagatagatagataggaagaaataagtaagaataacaagcaaacaacaaaagccgggaagcgccgaatcgatcgtgatctaaagatcctctacaccaacaaaattattatgtttattaaactgacgatcgaatggtacctctgatttctctttccattgctctttaacctaatttcctgtgtccaattattcccttccccctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 40 - RNA, 1268 nt, synthetic construct
  source 1..1268: mol_type = other RNA
tcagatcgctagcgtagcccggctagatagatagataggaagaaataagtaagaataacaagcaaacaacaaaagccgggaagcgccgaatcgatcgtgatctaaagatcctctacaccaacaaaattattatgtttattaaactgacgatcgaatggtacctctgatttctctttccattgctctttaacctaatttcctgtgtccaattattcccttccccctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgc

SEQ ID NO: 41 - DNA, 291 bp, synthetic construct
  source 1..291: mol_type = other DNA
gctagcgtagcccggcaacagttcctttatacaaattcaccagatgatacaaaacatatttttttagccgggaagcgccgaatcgatcgtgatctaaagatcctctacaccaacaaaattattatgtttattaaactgacgatcgaatggtacctgtccactttctttcttgatccttatattcctgtgccccttttccttcctccccacagtggacatttccggtaattctccacctctctaaacctaaattaaattttcttctctaaacctctttcctagttcctcagc

SEQ ID NO: 42 - DNA, 50 bp, synthetic construct; identical to SEQ ID NO: 15
  source 1..50: mol_type = other DNA

SEQ ID NO: 43 - DNA, 50 bp, synthetic construct; identical to SEQ ID NO: 16
  source 1..50: mol_type = other DNA

SEQ ID NO: 44 - DNA, 50 bp, synthetic construct
  source 1..50: mol_type = other DNA
tttctttcttgatccttatattcctgtgccccttttccttcctccccaca

SEQ ID NO: 45 - DNA, 50 bp, synthetic construct
  source 1..50: mol_type = other DNA
acctctctaaacctaaattaaattttcttctctaaacctctttcctagtt

SEQ ID NO: 46 - DNA, 10 bp, synthetic construct
  source 1..10: mol_type = other DNA
gtagcccggc

SEQ ID NO: 47 - DNA, 24 bp, synthetic construct
  source 1..24: mol_type = other DNA
gccgggaagcgccgaatcgatcgt

SEQ ID NO: 48 - DNA, 21 bp, synthetic construct
  source 1..21: mol_type = other DNA
gatcgaatggtacctgtccac

SEQ ID NO: 49 - DNA, 23 bp, synthetic construct
  source 1..23: mol_type = other DNA
gtggacatttccggtaattctcc

SEQ ID NO: 50 - DNA, 2424 bp, synthetic construct
  source 1..2424: mol_type = other DNA
actagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagcgtagcccggcaacagttcctttatacaaattcaccagatgatacaaaacatatttttttagccgggaagcgccgaatcgatcgtgatctaaagatcctctacaccaacaaaattattatgtttattaaactgacgatcgaatggtacctgtccactttctttcttgatccttatattcctgtgccccttttccttcctccccacagtggacatttccggtaattctccacctctctaaacctaaattaaattttcttctctaaacctctttcctagttcctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctgcttcgtacccctgccatcaacacgcgtctgcgttcgaccaggcggcgcgatcacggggacacagcaaccgacggacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 51 - DNA, 2661 bp, synthetic construct
  source 1..2661: mol_type = other DNA
cgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcactagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagcgtagcccggcaacagttcctttatacaaattcaccagatgatacaaaacatatttttttagccgggaagcgccgaatcgatcgtgatctaaagatcctctacaccaacaaaattattatgtttattaaactgacgatcgaatggtacctgtccactttctttcttgatccttatattcctgtgccccttttccttcctccccacagtggacatttccggtaattctccacctctctaaacctaaattaaattttcttctctaaacctctttcctagttcctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctgcttcgtacccctgccatcaacacgcgtctgcgttcgaccaggcggcgcgatcacggggacacagcaaccgacggacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 52 - RNA, 1784 nt, synthetic construct
  source 1..1784: mol_type = other RNA
tcagatcgctagcgtagcccggcaacagttcctttatacaaattcaccagatgatacaaaacatatttttttagccgggaagcgccgaatcgatcgtgatctaaagatcctctacaccaacaaaattattatgtttattaaactgacgatcgaatggtacctgtccactttctttcttgatccttatattcctgtgccccttttccttcctccccacagtggacatttccggtaattctccacctctctaaacctaaattaaattttcttctctaaacctctttcctagttcctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctgcttcgtacccctgccatcaacacgcgtctgcgttcgaccaggcggcgcgatcacggggacacagcaaccgacggacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgc

SEQ ID NO: 53 - DNA, 1977 bp, synthetic construct
  source 1..1977: mol_type = other DNA
actagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagcgtagcccggcaacagttcctttatacaaattcaccagatgatacaaaacatatttttttagccgggaagcgccgaatcgatcgtgatctaaagatcctctacaccaacaaaattattatgtttattaaactgacgatcgaatggtacctgtccactttctttcttgatccttatattcctgtgccccttttccttcctccccacagtggacatttccggtaattctccacctctctaaacctaaattaaattttcttctctaaacctctttcctagttcctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 54 - DNA, 2214 bp, synthetic construct
  source 1..2214: mol_type = other DNA
cgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcactagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagcgtagcccggcaacagttcctttatacaaattcaccagatgatacaaaacatatttttttagccgggaagcgccgaatcgatcgtgatctaaagatcctctacaccaacaaaattattatgtttattaaactgacgatcgaatggtacctgtccactttctttcttgatccttatattcctgtgccccttttccttcctccccacagtggacatttccggtaattctccacctctctaaacctaaattaaattttcttctctaaacctctttcctagttcctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 55 - RNA, 1337 nt, synthetic construct
  source 1..1337: mol_type = other RNA
tcagatcgctagcgtagcccggcaacagttcctttatacaaattcaccagatgatacaaaacatatttttttagccgggaagcgccgaatcgatcgtgatctaaagatcctctacaccaacaaaattattatgtttattaaactgacgatcgaatggtacctgtccactttctttcttgatccttatattcctgtgccccttttccttcctccccacagtggacatttccggtaattctccacctctctaaacctaaattaaattttcttctctaaacctctttcctagttcctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgc

SEQ ID NO: 56 - DNA, 312 bp, synthetic construct
  source 1..312: mol_type = other DNA
gctagcctggaaaaaaagggaaaaaaagtagcccggcaacagttcctttatacaaattcaccagatgatacaaaacatatttttttagccgggaagcgccgaatcgatcgtgatctaaagatcctctacaccaacaaaattattatgtttattaaactgacgatcgaatggtacctgtccactttctttcttgatccttatattcctgtgccccttttccttcctccccacagtggacatttccggtaattctccacctctctaaacctaaattaaattttcttctctaaacctctttcctagttcctcagc

SEQ ID NO: 57 - DNA, 2445 bp, synthetic construct
  source 1..2445: mol_type = other DNA
actagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagcctggaaaaaaagggaaaaaaagtagcccggcaacagttcctttatacaaattcaccagatgatacaaaacatatttttttagccgggaagcgccgaatcgatcgtgatctaaagatcctctacaccaacaaaattattatgtttattaaactgacgatcgaatggtacctgtccactttctttcttgatccttatattcctgtgccccttttccttcctccccacagtggacatttccggtaattctccacctctctaaacctaaattaaattttcttctctaaacctctttcctagttcctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctgcttcgtacccctgccatcaacacgcgtctgcgttcgaccaggcggcgcgatcacggggacacagcaaccgacggacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 58 - DNA, 2682 bp, synthetic construct
  source 1..2682: mol_type = other DNA
cgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcactagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagcctggaaaaaaagggaaaaaaagtagcccggcaacagttcctttatacaaattcaccagatgatacaaaacatatttttttagccgggaagcgccgaatcgatcgtgatctaaagatcctctacaccaacaaaattattatgtttattaaactgacgatcgaatggtacctgtccactttctttcttgatccttatattcctgtgccccttttccttcctccccacagtggacatttccggtaattctccacctctctaaacctaaattaaattttcttctctaaacctctttcctagttcctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctgcttcgtacccctgccatcaacacgcgtctgcgttcgaccaggcggcgcgatcacggggacacagcaaccgacggacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 59 - RNA, 1805 nt, synthetic construct
  source 1..1805: mol_type = other RNA
tcagatcgctagcctggaaaaaaagggaaaaaaagtagcccggcaacagttcctttatacaaattcaccagatgatacaaaacatatttttttagccgggaagcgccgaatcgatcgtgatctaaagatcctctacaccaacaaaattattatgtttattaaactgacgatcgaatggtacctgtccactttctttcttgatccttatattcctgtgccccttttccttcctccccacagtggacatttccggtaattctccacctctctaaacctaaattaaattttcttctctaaacctctttcctagttcctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctgcttcgtacccctgccatcaacacgcgtctgcgttcgaccaggcggcgcgatcacggggacacagcaaccgacggacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgc

SEQ ID NO: 60 - DNA, 1998 bp, synthetic construct
  source 1..1998: mol_type = other DNA
actagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagcctggaaaaaaagggaaaaaaagtagcccggcaacagttcctttatacaaattcaccagatgatacaaaacatatttttttagccgggaagcgccgaatcgatcgtgatctaaagatcctctacaccaacaaaattattatgtttattaaactgacgatcgaatggtacctgtccactttctttcttgatccttatattcctgtgccccttttccttcctccccacagtggacatttccggtaattctccacctctctaaacctaaattaaattttcttctctaaacctctttcctagttcctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 61 - DNA, 2235 bp, synthetic construct
  source 1..2235: mol_type = other DNA
cgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcactagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagcctggaaaaaaagggaaaaaaagtagcccggcaacagttcctttatacaaattcaccagatgatacaaaacatatttttttagccgggaagcgccgaatcgatcgtgatctaaagatcctctacaccaacaaaattattatgtttattaaactgacgatcgaatggtacctgtccactttctttcttgatccttatattcctgtgccccttttccttcctccccacagtggacatttccggtaattctccacctctctaaacctaaattaaattttcttctctaaacctctttcctagttcctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 62 - RNA, 1358 nt, synthetic construct
  source 1..1358: mol_type = other RNA
tcagatcgctagcctggaaaaaaagggaaaaaaagtagcccggcaacagttcctttatacaaattcaccagatgatacaaaacatatttttttagccgggaagcgccgaatcgatcgtgatctaaagatcctctacaccaacaaaattattatgtttattaaactgacgatcgaatggtacctgtccactttctttcttgatccttatattcctgtgccccttttccttcctccccacagtggacatttccggtaattctccacctctctaaacctaaattaaattttcttctctaaacctctttcctagttcctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgc

SEQ ID NO: 63 - DNA, 2439 bp, synthetic construct
  source 1..2439: mol_type = other DNA
actagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagcctggaaaaaaagggaagtagcccggcaacagttcctttatacaaattcaccagatgatacaaaacatatttttttagccgaaaaacgatcgtcgatcgtgatctaaagatcctctacaccaacaaaattattatgtttattaaactgacgatcgaatggtacctgtccactttctttcttgatccttatattcctgtgccccttttccttcctccccacagtggacatttccggtaattctccacctctctaaacctaaattaaattttcttctctaaacctctttcctagttcctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctgcttcgtacccctgccatcaacacgcgtctgcgttcgaccaggcggcgcgatcacggggacacagcaaccgacggacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 64 - DNA, 2676 bp, synthetic construct
  source 1..2676: mol_type = other DNA
cgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcactagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagcctggaaaaaaagggaagtagcccggcaacagttcctttatacaaattcaccagatgatacaaaacatatttttttagccgaaaaacgatcgtcgatcgtgatctaaagatcctctacaccaacaaaattattatgtttattaaactgacgatcgaatggtacctgtccactttctttcttgatccttatattcctgtgccccttttccttcctccccacagtggacatttccggtaattctccacctctctaaacctaaattaaattttcttctctaaacctctttcctagttcctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctgcttcgtacccctgccatcaacacgcgtctgcgttcgaccaggcggcgcgatcacggggacacagcaaccgacggacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 65 - RNA, 1799 nt, synthetic construct
  source 1..1799: mol_type = other RNA
tcagatcgctagcctggaaaaaaagggaagtagcccggcaacagttcctttatacaaattcaccagatgatacaaaacatatttttttagccgaaaaacgatcgtcgatcgtgatctaaagatcctctacaccaacaaaattattatgtttattaaactgacgatcgaatggtacctgtccactttctttcttgatccttatattcctgtgccccttttccttcctccccacagtggacatttccggtaattctccacctctctaaacctaaattaaattttcttctctaaacctctttcctagttcctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctgcttcgtacccctgccatcaacacgcgtctgcgttcgaccaggcggcgcgatcacggggacacagcaaccgacggacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgc

SEQ ID NO: 66 - DNA, 1992 bp, synthetic construct
  source 1..1992: mol_type = other DNA
actagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagcctggaaaaaaagggaagtagcccggcaacagttcctttatacaaattcaccagatgatacaaaacatatttttttagccgaaaaacgatcgtcgatcgtgatctaaagatcctctacaccaacaaaattattatgtttattaaactgacgatcgaatggtacctgtccactttctttcttgatccttatattcctgtgccccttttccttcctccccacagtggacatttccggtaattctccacctctctaaacctaaattaaattttcttctctaaacctctttcctagttcctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 67 - DNA, 2229 bp, synthetic construct
  source 1..2229: mol_type = other DNA
cgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcactagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagcctggaaaaaaagggaagtagcccggcaacagttcctttatacaaattcaccagatgatacaaaacatatttttttagccgaaaaacgatcgtcgatcgtgatctaaagatcctctacaccaacaaaattattatgtttattaaactgacgatcgaatggtacctgtccactttctttcttgatccttatattcctgtgccccttttccttcctccccacagtggacatttccggtaattctccacctctctaaacctaaattaaattttcttctctaaacctctttcctagttcctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 68 - RNA, 1352 nt, synthetic construct
  source 1..1352: mol_type = other RNA
tcagatcgctagcctggaaaaaaagggaagtagcccggcaacagttcctttatacaaattcaccagatgatacaaaacatatttttttagccgaaaaacgatcgtcgatcgtgatctaaagatcctctacaccaacaaaattattatgtttattaaactgacgatcgaatggtacctgtccactttctttcttgatccttatattcctgtgccccttttccttcctccccacagtggacatttccggtaattctccacctctctaaacctaaattaaattttcttctctaaacctctttcctagttcctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgc

SEQ ID NO: 69 - DNA, 365 bp, synthetic construct
  source 1..365: mol_type = other DNA
gctagcgtagcccggcggtagatagatagataggaagaaataagtaagaataacaagcaaacaacaaaaccgccgggaagcgccgaatgatcgtttctctttccattgctctttaacctaatttcctgtgtccaattattcccttccccgatcaatcctgtccaccaaattcccatcttaccacttttccttttacactttcttaatcattttgcatgttttcttctcccagatggtggacaacctctctaaacctaaattaaattttcttctctaaacctctttcctagtttctcccgcgcaattcggtctctcttatcttctaacatcctatcttattctgtctgtccccacctgtcctcagc

SEQ ID NO: 70 - DNA, 12 bp, synthetic construct
  source 1..12: mol_type = other DNA
gtagcccggcgg

SEQ ID NO: 71 - DNA, 24 bp, synthetic construct
  source 1..24: mol_type = other DNA
ccgccgggaagcgccgaatgatcg

SEQ ID NO: 72 - DNA, 18 bp, synthetic construct
  source 1..18: mol_type = other DNA
cgatcaatcctgtccacc

SEQ ID NO: 73 - skipped

SEQ ID NO: 74 - DNA, 10 bp, synthetic construct
  source 1..10: mol_type = other DNA
tctcccgcgc

SEQ ID NO: 75 - DNA, 51 bp, synthetic construct; identical to SEQ ID NO: 29
  source 1..51: mol_type = other DNA

SEQ ID NO: 76 - DNA, 55 bp, synthetic construct; identical to SEQ ID NO: 31
  source 1..55: mol_type = other DNA

SEQ ID NO: 77 - DNA, 69 bp, synthetic construct
  source 1..69: mol_type = other DNA
aaattcccatcttaccacttttccttttacactttcttaatcattttgcatgttttcttctcccagatg

SEQ ID NO: 78 - DNA, 50 bp, synthetic construct; identical to SEQ ID NO: 45
  source 1..50: mol_type = other DNA

SEQ ID NO: 79 - DNA, 56 bp, synthetic construct
  source 1..56: mol_type = other DNA
aattcggtctctcttatcttctaacatcctatcttattctgtctgtccccacctgt

SEQ ID NO: 80 - DNA, 2498 bp, synthetic construct
  source 1..2498: mol_type = other DNA
actagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagcgtagcccggcggtagatagatagataggaagaaataagtaagaataacaagcaaacaacaaaaccgccgggaagcgccgaatgatcgtttctctttccattgctctttaacctaatttcctgtgtccaattattcccttccccgatcaatcctgtccaccaaattcccatcttaccacttttccttttacactttcttaatcattttgcatgttttcttctcccagatggtggacaacctctctaaacctaaattaaattttcttctctaaacctctttcctagtttctcccgcgcaattcggtctctcttatcttctaacatcctatcttattctgtctgtccccacctgtcctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctgcttcgtacccctgccatcaacacgcgtctgcgttcgaccaggcggcgcgatcacggggacacagcaaccgacggacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 81 - DNA, 2735 bp, synthetic construct
  source 1..2735: mol_type = other DNA
cgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcactagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagcgtagcccggcggtagatagatagataggaagaaataagtaagaataacaagcaaacaacaaaaccgccgggaagcgccgaatgatcgtttctctttccattgctctttaacctaatttcctgtgtccaattattcccttccccgatcaatcctgtccaccaaattcccatcttaccacttttccttttacactttcttaatcattttgcatgttttcttctcccagatggtggacaacctctctaaacctaaattaaattttcttctctaaacctctttcctagtttctcccgcgcaattcggtctctcttatcttctaacatcctatcttattctgtctgtccccacctgtcctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctgcttcgtacccctgccatcaacacgcgtctgcgttcgaccaggcggcgcgatcacggggacacagcaaccgacggacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 82 - RNA, 1858 nt, synthetic construct
  source 1..1858: mol_type = other RNA
tcagatcgctagcgtagcccggcggtagatagatagataggaagaaataagtaagaataacaagcaaacaacaaaaccgccgggaagcgccgaatgatcgtttctctttccattgctctttaacctaatttcctgtgtccaattattcccttccccgatcaatcctgtccaccaaattcccatcttaccacttttccttttacactttcttaatcattttgcatgttttcttctcccagatggtggacaacctctctaaacctaaattaaattttcttctctaaacctctttcctagtttctcccgcgcaattcggtctctcttatcttctaacatcctatcttattctgtctgtccccacctgtcctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctgcttcgtacccctgccatcaacacgcgtctgcgttcgaccaggcggcgcgatcacggggacacagcaaccgacggacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgc

SEQ ID NO: 83 - DNA, 2051 bp, synthetic construct
  source 1..2051: mol_type = other DNA
actagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagcgtagcccggcggtagatagatagataggaagaaataagtaagaataacaagcaaacaacaaaaccgccgggaagcgccgaatgatcgtttctctttccattgctctttaacctaatttcctgtgtccaattattcccttccccgatcaatcctgtccaccaaattcccatcttaccacttttccttttacactttcttaatcattttgcatgttttcttctcccagatggtggacaacctctctaaacctaaattaaattttcttctctaaacctctttcctagtttctcccgcgcaattcggtctctcttatcttctaacatcctatcttattctgtctgtccccacctgtcctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 84 - DNA, 2288 bp, synthetic construct
  source 1..2288: mol_type = other DNA
cgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcactagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagcgtagcccggcggtagatagatagataggaagaaataagtaagaataacaagcaaacaacaaaaccgccgggaagcgccgaatgatcgtttctctttccattgctctttaacctaatttcctgtgtccaattattcccttccccgatcaatcctgtccaccaaattcccatcttaccacttttccttttacactttcttaatcattttgcatgttttcttctcccagatggtggacaacctctctaaacctaaattaaattttcttctctaaacctctttcctagtttctcccgcgcaattcggtctctcttatcttctaacatcctatcttattctgtctgtccccacctgtcctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 85 - RNA, 1411 nt, synthetic construct
  source 1..1411: mol_type = other RNA
tcagatcgctagcgtagcccggcggtagatagatagataggaagaaataagtaagaataacaagcaaacaacaaaaccgccgggaagcgccgaatgatcgtttctctttccattgctctttaacctaatttcctgtgtccaattattcccttccccgatcaatcctgtccaccaaattcccatcttaccacttttccttttacactttcttaatcattttgcatgttttcttctcccagatggtggacaacctctctaaacctaaattaaattttcttctctaaacctctttcctagtttctcccgcgcaattcggtctctcttatcttctaacatcctatcttattctgtctgtccccacctgtcctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgc

SEQ ID NO: 86 - DNA, 143 bp, synthetic construct
  source 1..143: mol_type = other DNA
gctagcggaaagaggaattagaggacaaacgggcaacaaacaatgatagtaaagaagaaccaacaagaagaaaaggcatagcagcaaaatgaagaggaagatgataaaacgtctttcctcagtccggtacctctgacctcagc

SEQ ID NO: 87 - DNA, 98 bp, synthetic construct
  source 1..98: mol_type = other DNA
ggaattagaggacaaacgggcaacaaacaatgatagtaaagaagaaccaacaagaagaaaaggcatagcagcaaaatgaagaggaagatgataaaacg

SEQ ID NO: 88 - skipped

SEQ ID NO: 89 - DNA, 25 bp, synthetic construct
  source 1..25: mol_type = other DNA
tctttcctcagtccggtacctctga

SEQ ID NO: 90 - DNA, 1813 bp, synthetic construct
  source 1..1813: mol_type = other DNA
actagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagcggaaagaggaattagaggacaaacgggcaacaaacaatgatagtaaagaagaaccaacaagaagaaaaggcatagcagcaaaatgaagaggaagatgataaaacgtctttcctcagtccggtacctctgacctcagcccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 91 - DNA, 2050 bp, synthetic construct
  source 1..2050: mol_type = other DNA
cgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcactagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagcggaaagaggaattagaggacaaacgggcaacaaacaatgatagtaaagaagaaccaacaagaagaaaaggcatagcagcaaaatgaagaggaagatgataaaacgtctttcctcagtccggtacctctgacctcagcccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 92 - RNA, 1636 nt, synthetic construct
  source 1..1636: mol_type = other RNA
tcagatcgctagcggaaagaggaattagaggacaaacgggcaacaaacaatgatagtaaagaagaaccaacaagaagaaaaggcatagcagcaaaatgaagaggaagatgataaaacgtctttcctcagtccggtacctctgacctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctgcttcgtacccctgccatcaacacgcgtctgcgttcgaccaggcggcgcgatcacggggacacagcaaccgacggacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgc

SEQ ID NO: 93 - DNA, 136 bp, synthetic construct
  source 1..136: mol_type = other DNA
gctagcctggaaaaaaagggaaaaaaagctagcggaaagaggaattagaggacaaacgggcaacaaacaatgatagtaaagaagaaccaacaagaagaaaaggcatagcagcaaaatgaagaggaagatgataaaa

SEQ ID NO: 94 - DNA, 1840 bp, synthetic construct
  source 1..1840: mol_type = other DNA
actagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagcctggaaaaaaagggaaaaaaagctagcggaaagaggaattagaggacaaacgggcaacaaacaatgatagtaaagaagaaccaacaagaagaaaaggcatagcagcaaaatgaagaggaagatgataaaacgtctttcctcagtccggtacctctgacctcagcccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 95 - DNA, 2077 bp, synthetic construct
  source 1..2077: mol_type = other DNA
cgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcactagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagcctggaaaaaaagggaaaaaaagctagcggaaagaggaattagaggacaaacgggcaacaaacaatgatagtaaagaagaaccaacaagaagaaaaggcatagcagcaaaatgaagaggaagatgataaaacgtctttcctcagtccggtacctctgacctcagcccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 96 - RNA, 1663 nt, synthetic construct
  source 1..1663: mol_type = other RNA
tcagatcgctagcctggaaaaaaagggaaaaaaagctagcggaaagaggaattagaggacaaacgggcaacaaacaatgatagtaaagaagaaccaacaagaagaaaaggcatagcagcaaaatgaagaggaagatgataaaacgtctttcctcagtccggtacctctgacctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctgcttcgtacccctgccatcaacacgcgtctgcgttcgaccaggcggcgcgatcacggggacacagcaaccgacggacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgc

SEQ ID NO: 97 - DNA, 84 bp, synthetic construct
  source 1..84: mol_type = other DNA
gctagtctagaggtacctctgatttctctttccattgctctttaacctaatttcctgtgtccaattattcccttccccctcagc

SEQ ID NO: 98 - DNA, 55 bp, synthetic construct; identical to SEQ ID NO: 31
  source 1..55: mol_type = other DNA

SEQ ID NO: 99 - DNA, 17 bp, synthetic construct
  source 1..17: mol_type = other DNA
tctagaggtacctctga

SEQ ID NO: 100 - DNA, 1754 bp, synthetic construct
  source 1..1754: mol_type = other DNA
actagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagtctagaggtacctctgatttctctttccattgctctttaacctaatttcctgtgtccaattattcccttccccctcagcccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 101 - DNA, 1991 bp, synthetic construct
  source 1..1991: mol_type = other DNA
cgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcactagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagtctagaggtacctctgatttctctttccattgctctttaacctaatttcctgtgtccaattattcccttccccctcagcccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 102 - RNA, 1577 nt, synthetic construct
  source 1..1577: mol_type = other RNA
tcagatcgctagtctagaggtacctctgatttctctttccattgctctttaacctaatttcctgtgtccaattattcccttccccctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctgcttcgtacccctgccatcaacacgcgtctgcgttcgaccaggcggcgcgatcacggggacacagcaaccgacggacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgc

SEQ ID NO: 103 - DNA, 100 bp, synthetic construct
  source 1..100: mol_type = other DNA
gctagcctggaaaaaaagggaaaaaaaggtacctctgatttctctttccattgctctttaacctaatttcctgtgtccaattattcccttccccctcagc

SEQ ID NO: 104 - skipped

SEQ ID NO: 105 - DNA, 2007 bp, synthetic construct
  source 1..2007: mol_type = other DNA
cgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcactagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagcctggaaaaaaagggaaaaaaaggtacctctgatttctctttccattgctctttaacctaatttcctgtgtccaattattcccttccccctcagcccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 106 - RNA, 1593 nt, synthetic construct
  source 1..1593: mol_type = other RNA
tcagatcgctagcctggaaaaaaagggaaaaaaaggtacctctgatttctctttccattgctctttaacctaatttcctgtgtccaattattcccttccccctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctgcttcgtacccctgccatcaacacgcgtctgcgttcgaccaggcggcgcgatcacggggacacagcaaccgacggacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgc

SEQ ID NO: 107 - DNA, 191 bp, synthetic construct
  source 1..191: mol_type = other DNA
gctagaggaattagaggacaaacgggcaacatacaatgatagtaaagaagaaccaacaagaagaaaaggcatagcagcaaaatgaagaggaagatgataaaacgtctttcctcagtccggtacctctgatttctctttccattgctctttaacctaatttcctgtgtccaattattcccttccccctcagc

SEQ ID NO: 108 - DNA, 98 bp, synthetic construct; identical to SEQ ID NO: 87
  source 1..98: mol_type = other DNA

SEQ ID NO: 109 - DNA, 55 bp, synthetic construct; identical to SEQ ID NO: 31
  source 1..55: mol_type = other DNA

SEQ ID NO: 110 - DNA, 24 bp, synthetic construct
  source 1..24: mol_type = other DNA
tctttcctcagtccggtacctctg

SEQ ID NO: 111 - DNA, 1861 bp, synthetic construct
  source 1..1861: mol_type = other DNA
actagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagaggaattagaggacaaacgggcaacatacaatgatagtaaagaagaaccaacaagaagaaaaggcatagcagcaaaatgaagaggaagatgataaaacgtctttcctcagtccggtacctctgatttctctttccattgctctttaacctaatttcctgtgtccaattattcccttccccctcagcccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 112 - DNA, 2098 bp, synthetic construct
  source 1..2098: mol_type = other DNA
cgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcactagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagaggaattagaggacaaacgggcaacatacaatgatagtaaagaagaaccaacaagaagaaaaggcatagcagcaaaatgaagaggaagatgataaaacgtctttcctcagtccggtacctctgatttctctttccattgctctttaacctaatttcctgtgtccaattattcccttccccctcagcccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 113 - RNA, 1684 nt, synthetic construct
  source 1..1684: mol_type = other RNA
tcagatcgctagaggaattagaggacaaacgggcaacatacaatgatagtaaagaagaaccaacaagaagaaaaggcatagcagcaaaatgaagaggaagatgataaaacgtctttcctcagtccggtacctctgatttctctttccattgctctttaacctaatttcctgtgtccaattattcccttccccctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctgcttcgtacccctgccatcaacacgcgtctgcgttcgaccaggcggcgcgatcacggggacacagcaaccgacggacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgc

SEQ ID NO: 114 - DNA, 218 bp, synthetic construct
  source 1..218: mol_type = other DNA
gctagactggaaaaaaagggaaaaaaatctagaggaattagaggacaaacgggcaacatacaatgatagtaaagaagaaccaacaagaagaaaaggcatagcagcaaaatgaagaggaagatgataaaacgtctttcctcagtccggtacctctgatttctctttccattgctctttaacctaatttcctgtgtccaattattcccttccccctcagc

SEQ ID NO: 115 - DNA, 1888 bp, synthetic construct
  source 1..1888: mol_type = other DNA
actagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagactggaaaaaaagggaaaaaaatctagaggaattagaggacaaacgggcaacatacaatgatagtaaagaagaaccaacaagaagaaaaggcatagcagcaaaatgaagaggaagatgataaaacgtctttcctcagtccggtacctctgatttctctttccattgctctttaacctaatttcctgtgtccaattattcccttccccctcagcccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 116 - DNA, 2125 bp, synthetic construct
  source 1..2125: mol_type = other DNA
cgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcactagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagactggaaaaaaagggaaaaaaatctagaggaattagaggacaaacgggcaacatacaatgatagtaaagaagaaccaacaagaagaaaaggcatagcagcaaaatgaagaggaagatgataaaacgtctttcctcagtccggtacctctgatttctctttccattgctctttaacctaatttcctgtgtccaattattcccttccccctcagcccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 117 - RNA, 1711 nt, synthetic construct
  source 1..1711: mol_type = other RNA
tcagatcgctagactggaaaaaaagggaaaaaaatctagaggaattagaggacaaacgggcaacatacaatgatagtaaagaagaaccaacaagaagaaaaggcatagcagcaaaatgaagaggaagatgataaaacgtctttcctcagtccggtacctctgatttctctttccattgctctttaacctaatttcctgtgtccaattattcccttccccctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctgcttcgtacccctgccatcaacacgcgtctgcgttcgaccaggcggcgcgatcacggggacacagcaaccgacggacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgc

SEQ ID NO: 118 - DNA, 218 bp, synthetic construct
  source 1..218: mol_type = other DNA
gctagaggaaagaggaattagaggacaaacgggcaacaaacaatgatagtaaagaagaaccaacaagaagaaaaggcatagcagcaaaatgaagaggaagatgataaaacgtctttcctcagtccggtacctctgattcctcgggccacctctctaaaaataaattaaattttcttctctaaacctctttcctagttggccctttctttcccctcagc

SEQ ID NO: 119 - DNA, 98 bp, synthetic construct; identical to SEQ ID NO: 87
  source 1..98: mol_type = other DNA

SEQ ID NO: 120 - DNA, 60 bp, synthetic construct
  source 1..60: mol_type = other DNA
gggccacctctctaaaaataaattaaattttcttctctaaacctctttcctagttggccc

SEQ ID NO: 121 - DNA, 1888 bp, synthetic construct
  source 1..1888: mol_type = other DNA
actagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagaggaaagaggaattagaggacaaacgggcaacaaacaatgatagtaaagaagaaccaacaagaagaaaaggcatagcagcaaaatgaagaggaagatgataaaacgtctttcctcagtccggtacctctgattcctcgggccacctctctaaaaataaattaaattttcttctctaaacctctttcctagttggccctttctttcccctcagcccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 122 - DNA, 2125 bp, synthetic construct
  source 1..2125: mol_type = other DNA
cgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcactagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagaggaaagaggaattagaggacaaacgggcaacaaacaatgatagtaaagaagaaccaacaagaagaaaaggcatagcagcaaaatgaagaggaagatgataaaacgtctttcctcagtccggtacctctgattcctcgggccacctctctaaaaataaattaaattttcttctctaaacctctttcctagttggccctttctttcccctcagcccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 123 - RNA, 1711 nt, synthetic construct
  source 1..1711: mol_type = other RNA
tcagatcgctagaggaaagaggaattagaggacaaacgggcaacaaacaatgatagtaaagaagaaccaacaagaagaaaaggcatagcagcaaaatgaagaggaagatgataaaacgtctttcctcagtccggtacctctgattcctcgggccacctctctaaaaataaattaaattttcttctctaaacctctttcctagttggccctttctttcccctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctgcttcgtacccctgccatcaacacgcgtctgcgttcgaccaggcggcgcgatcacggggacacagcaaccgacggacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgc

SEQ ID NO: 124 - DNA, 245 bp, synthetic construct
  source 1..245: mol_type = other DNA
gctagactggaaaaaaagggaaaaaaatctagaggaaagaggaattagaggacaaacgggcaacaaacaatgatagtaaagaagaaccaacaagaagaaaaggcatagcagcaaaatgaagaggaagatgataaaacgtctttcctcagtccggtacctctgattcctcgggccacctctctaaaaataaattaaattttcttctctaaacctctttcctagttggccctttctttcccctcagc

SEQ ID NO: 125 - DNA, 1915 bp, synthetic construct
  source 1..1915: mol_type = other DNA
actagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagactggaaaaaaagggaaaaaaatctagaggaaagaggaattagaggacaaacgggcaacaaacaatgatagtaaagaagaaccaacaagaagaaaaggcatagcagcaaaatgaagaggaagatgataaaacgtctttcctcagtccggtacctctgattcctcgggccacctctctaaaaataaattaaattttcttctctaaacctctttcctagttggccctttctttcccctcagcccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 126 - DNA, 2152 bp, synthetic construct
  source 1..2152: mol_type = other DNA
cgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcactagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagactggaaaaaaagggaaaaaaatctagaggaaagaggaattagaggacaaacgggcaacaaacaatgatagtaaagaagaaccaacaagaagaaaaggcatagcagcaaaatgaagaggaagatgataaaacgtctttcctcagtccggtacctctgattcctcgggccacctctctaaaaataaattaaattttcttctctaaacctctttcctagttggccctttctttcccctcagcccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 127 - RNA, 1738 nt, synthetic construct
  source 1..1738: mol_type = other RNA
tcagatcgctagactggaaaaaaagggaaaaaaatctagaggaaagaggaattagaggacaaacgggcaacaaacaatgatagtaaagaagaaccaacaagaagaaaaggcatagcagcaaaatgaagaggaagatgataaaacgtctttcctcagtccggtacctctgattcctcgggccacctctctaaaaataaattaaattttcttctctaaacctctttcctagttggccctttctttcccctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctgcttcgtacccctgccatcaacacgcgtctgcgttcgaccaggcggcgcgatcacggggacacagcaaccgacggacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgc

SEQ ID NO: 128 - DNA, 1931 bp, synthetic construct
  source 1..1931: mol_type = other DNA
actagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagcctggaaaaaaagggaaaaaaatctagaggaaagaggaattagaggacaaacgggcaacaaacaatgatagtaaagaagaaccaacaagaagaaaaggcatagcagcaaaatgaagaggaagatgataaaacgtctttcctcagtccggtacctctgattcctcgggccacctctctaaaaataaattaaattttcttctctaaacctctttcctagttggccctttctttcccctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 129 - DNA, 2168 bp, synthetic construct
  source 1..2168: mol_type = other DNA
cgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcactagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagcctggaaaaaaagggaaaaaaatctagaggaaagaggaattagaggacaaacgggcaacaaacaatgatagtaaagaagaaccaacaagaagaaaaggcatagcagcaaaatgaagaggaagatgataaaacgtctttcctcagtccggtacctctgattcctcgggccacctctctaaaaataaattaaattttcttctctaaacctctttcctagttggccctttctttcccctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 130 - RNA, 1291 nt, synthetic construct
  source 1..1291: mol_type = other RNA
tcagatcgctagcctggaaaaaaagggaaaaaaatctagaggaaagaggaattagaggacaaacgggcaacaaacaatgatagtaaagaagaaccaacaagaagaaaaggcatagcagcaaaatgaagaggaagatgataaaacgtctttcctcagtccggtacctctgattcctcgggccacctctctaaaaataaattaaattttcttctctaaacctctttcctagttggccctttctttcccctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgc

SEQ ID NO: 131 - DNA, 143 bp, synthetic construct
  source 1..143: mol_type = other DNA
gctagcacctctctaaacctaaattaaattttcttctctaaacctctttcctagttggactgacgtcactcagtcctcctgaattctttctttcttgatccttatattcctgtgccccttttccttcctccccacacctcagc

SEQ ID NO: 132 - DNA, 50 bp, synthetic construct; identical to SEQ ID NO: 45
  source 1..50: mol_type = other DNA

SEQ ID NO: 133 - DNA, 50 bp, synthetic construct; identical to SEQ ID NO: 44
  source 1..50: mol_type = other DNA

SEQ ID NO: 134 - DNA, 1829 bp, synthetic construct
  source 1..1829: mol_type = other DNA
actagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagcacctctctaaacctaaattaaattttcttctctaaacctctttcctagttggactgacgtcactcagtcctcctgaattctttctttcttgatccttatattcctgtgccccttttccttcctccccacacctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 135 - DNA, 2066 bp, synthetic construct
  source 1..2066: mol_type = other DNA
cgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcactagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagcacctctctaaacctaaattaaattttcttctctaaacctctttcctagttggactgacgtcactcagtcctcctgaattctttctttcttgatccttatattcctgtgccccttttccttcctccccacacctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 136 - RNA, 1189 nt, synthetic construct
  source 1..1189: mol_type = other RNA
tcagatcgctagcacctctctaaacctaaattaaattttcttctctaaacctctttcctagttggactgacgtcactcagtcctcctgaattctttctttcttgatccttatattcctgtgccccttttccttcctccccacacctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgc

SEQ ID NO: 137 - DNA, 583 bp, synthetic construct
  source 1..583: mol_type = other DNA
gctagctttgtttccagcctccggcatagatggttagattctttttttatttttttttttgtggcctccctgctgagcgaatttgtttacagatttttatctgaatatcatctttattatctcacataccatgggtcgctcggcaagcccggcgcctagaaaaggcaaaaagacaataacaaaacaaaacaaaaaaagcaaaaactaaaaacacgcacatacaaatgaaacaattatcccgccgggctattctccttcaccccttacttacttttgattcttccagatgtctactctgaaacctctttgctacctttcctttcactcaccacctcattcagcggcgattgcagctgcgtgcttttctttcattaacaaacagctccacctttatttctcctttcttttcactgacctttcattttctcctgtgttccctattcttaggcagctgcgttcgccgctgatgattcgacccctacactgggaaccaccttatgcctccggttcggccctaaaaagacaaataataaaataaaataaaataaaataaaaaaaaataaaataaatttaattcctcagc

SEQ ID NO: 138 - DNA, 89 bp, synthetic construct
  source 1..89: mol_type = other DNA
attctccttcaccccttacttacttttgattcttccagatgtctactctgaaacctctttgctacctttcctttcactcaccacctcat

SEQ ID NO: 139 - DNA, 109 bp, synthetic construct
  source 1..109: mol_type = other DNA
tgattcgacccctacactgggaaccaccttatgcctccggttcggccctaaaaagacaaataataaaataaaataaaataaaataaaaaaaaataaaataaatttaatt

SEQ ID NO: 140 - DNA, 90 bp, synthetic construct
  source 1..90: mol_type = other DNA
gtgcttttctttcattaacaaacagctccacctttatttctcctttcttttcactgacctttcattttctcctgtgttccctattcttag

SEQ ID NO: 141 - DNA, 49 bp, synthetic construct
  source 1..49: mol_type = other DNA
ccagcctccggcatagatggttagattctttttttatttttttttttgt

SEQ ID NO: 142 - DNA, 54 bp, synthetic construct
  source 1..54: mol_type = other DNA
tttgtttacagatttttatctgaatatcatctttattatctcacataccatggg

SEQ ID NO: 143 - DNA, 85 bp, synthetic construct
  source 1..85: mol_type = other DNA
ctagaaaaggcaaaaagacaataacaaaacaaaacaaaaaaagcaaaaactaaaaacacgcacatacaaatgaaacaattatccc

SEQ ID NO: 144 - skipped

SEQ ID NO: 145 - DNA, 19 bp, synthetic construct
  source 1..19: mol_type = other DNA
ggcctccctgctgagcgaa

SEQ ID NO: 146 - DNA, 21 bp, synthetic construct
  source 1..21: mol_type = other DNA
tcgctcggcaagcccggcgcc

SEQ ID NO: 147 - DNA, 20 bp, synthetic construct
  source 1..20: mol_type = other DNA
tcagcggcgattgcagctgc

SEQ ID NO: 148 - DNA, 2253 bp, synthetic construct
  source 1..2253: mol_type = other DNA
actagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagctttgtttccagcctccggcatagatggttagattctttttttatttttttttttgtggcctccctgctgagcgaatttgtttacagatttttatctgaatatcatctttattatctcacataccatgggtcgctcggcaagcccggcgcctagaaaaggcaaaaagacaataacaaaacaaaacaaaaaaagcaaaaactaaaaacacgcacatacaaatgaaacaattatcccgccgggctattctccttcaccccttacttacttttgattcttccagatgtctactctgaaacctctttgctacctttcctttcactcaccacctcattcagcggcgattgcagctgcgtgcttttctttcattaacaaacagctccacctttatttctcctttcttttcactgacctttcattttctcctgtgttccctattcttaggcagctgcgttcgccgctgatgattcgacccctacactgggaaccaccttatgcctccggttcggccctaaaaagacaaataataaaataaaataaaataaaataaaaaaaaataaaataaatttaattcctcagcccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 149 - DNA, 2490 bp, synthetic construct
  source 1..2490: mol_type = other DNA
cgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcactagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagctttgtttccagcctccggcatagatggttagattctttttttatttttttttttgtggcctccctgctgagcgaatttgtttacagatttttatctgaatatcatctttattatctcacataccatgggtcgctcggcaagcccggcgcctagaaaaggcaaaaagacaataacaaaacaaaacaaaaaaagcaaaaactaaaaacacgcacatacaaatgaaacaattatcccgccgggctattctccttcaccccttacttacttttgattcttccagatgtctactctgaaacctctttgctacctttcctttcactcaccacctcattcagcggcgattgcagctgcgtgcttttctttcattaacaaacagctccacctttatttctcctttcttttcactgacctttcattttctcctgtgttccctattcttaggcagctgcgttcgccgctgatgattcgacccctacactgggaaccaccttatgcctccggttcggccctaaaaagacaaataataaaataaaataaaataaaataaaaaaaaataaaataaatttaattcctcagcccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 150 - RNA, 2076 nt, synthetic construct
  source 1..2076: mol_type = other RNA
tcagatcgctagctttgtttccagcctccggcatagatggttagattctttttttatttttttttttgtggcctccctgctgagcgaatttgtttacagatttttatctgaatatcatctttattatctcacataccatgggtcgctcggcaagcccggcgcctagaaaaggcaaaaagacaataacaaaacaaaacaaaaaaagcaaaaactaaaaacacgcacatacaaatgaaacaattatcccgccgggctattctccttcaccccttacttacttttgattcttccagatgtctactctgaaacctctttgctacctttcctttcactcaccacctcattcagcggcgattgcagctgcgtgcttttctttcattaacaaacagctccacctttatttctcctttcttttcactgacctttcattttctcctgtgttccctattcttaggcagctgcgttcgccgctgatgattcgacccctacactgggaaccaccttatgcctccggttcggccctaaaaagacaaataataaaataaaataaaataaaataaaaaaaaataaaataaatttaattcctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctgcttcgtacccctgccatcaacacgcgtctgcgttcgaccaggcggcgcgatcacggggacacagcaaccgacggacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgc

SEQ ID NO: 151 - DNA, 2269 bp, synthetic construct
  source 1..2269: mol_type = other DNA
actagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagctttgtttccagcctccggcatagatggttagattctttttttatttttttttttgtggcctccctgctgagcgaatttgtttacagatttttatctgaatatcatctttattatctcacataccatgggtcgctcggcaagcccggcgcctagaaaaggcaaaaagacaataacaaaacaaaacaaaaaaagcaaaaactaaaaacacgcacatacaaatgaaacaattatcccgccgggctattctccttcaccccttacttacttttgattcttccagatgtctactctgaaacctctttgctacctttcctttcactcaccacctcattcagcggcgattgcagctgcgtgcttttctttcattaacaaacagctccacctttatttctcctttcttttcactgacctttcattttctcctgtgttccctattcttaggcagctgcgttcgccgctgatgattcgacccctacactgggaaccaccttatgcctccggttcggccctaaaaagacaaataataaaataaaataaaataaaataaaaaaaaataaaataaatttaattcctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 152 - DNA, 2506 bp, synthetic construct
  source 1..2506: mol_type = other DNA
cgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcactagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagctttgtttccagcctccggcatagatggttagattctttttttatttttttttttgtggcctccctgctgagcgaatttgtttacagatttttatctgaatatcatctttattatctcacataccatgggtcgctcggcaagcccggcgcctagaaaaggcaaaaagacaataacaaaacaaaacaaaaaaagcaaaaactaaaaacacgcacatacaaatgaaacaattatcccgccgggctattctccttcaccccttacttacttttgattcttccagatgtctactctgaaacctctttgctacctttcctttcactcaccacctcattcagcggcgattgcagctgcgtgcttttctttcattaacaaacagctccacctttatttctcctttcttttcactgacctttcattttctcctgtgttccctattcttaggcagctgcgttcgccgctgatgattcgacccctacactgggaaccaccttatgcctccggttcggccctaaaaagacaaataataaaataaaataaaataaaataaaaaaaaataaaataaatttaattcctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 153 - RNA, 1629 nt, synthetic construct
  source 1..1629: mol_type = other RNA
tcagatcgctagctttgtttccagcctccggcatagatggttagattctttttttatttttttttttgtggcctccctgctgagcgaatttgtttacagatttttatctgaatatcatctttattatctcacataccatgggtcgctcggcaagcccggcgcctagaaaaggcaaaaagacaataacaaaacaaaacaaaaaaagcaaaaactaaaaacacgcacatacaaatgaaacaattatcccgccgggctattctccttcaccccttacttacttttgattcttccagatgtctactctgaaacctctttgctacctttcctttcactcaccacctcattcagcggcgattgcagctgcgtgcttttctttcattaacaaacagctccacctttatttctcctttcttttcactgacctttcattttctcctgtgttccctattcttaggcagctgcgttcgccgctgatgattcgacccctacactgggaaccaccttatgcctccggttcggccctaaaaagacaaataataaaataaaataaaataaaataaaaaaaaataaaataaatttaattcctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgc

SEQ ID NO: 154 - DNA, 300 bp, synthetic construct
  source 1..300: mol_type = other DNA
gctagctccatatgccgcggagaggaccaaagaaatagcagagacaaaaaaaaaataaataaaaattaaaaaaaataaaaaaactaattcccggagcccggcgctatccacccatccaccatccatccccctgccagccagccacttatccatccaaccatcttcccatccacacccatccatctcctcatctatgcatccagcgccgggcttcagcggcgattgcagctgcccagttcttgcaatttcctataacacttacgtcaaaacatttccgcagctgcgttcgccgccctcagc

SEQ ID NO: 155 - DNA, 83 bp, synthetic construct
  source 1..83: mol_type = other DNA
tccatatgccgcggagaggaccaaagaaatagcagagacaaaaaaaaaataaataaaaattaaaaaaaataaaaaaactaatt

SEQ ID NO: 156 - DNA, 44 bp, Homo sapiens
  source 1..44: mol_type = other DNA
ccagttcttgcaatttcctataacacttacgtcaaaacatttcc

SEQ ID NO: 157 - DNA, 100 bp, Homo sapiens
  source 1..100: mol_type = other DNA
tatccacccatccaccatccatccccctgccagccagccacttatccatccaaccatcttcccatccacacccatccatctcctcatctatgcatccagc

SEQ ID NO: 158 - DNA, 15 bp, synthetic construct
  source 1..15: mol_type = other DNA
cccggagcccggcgc

SEQ ID NO: 159 - DNA, 28 bp, synthetic construct
  source 1..28: mol_type = other DNA
gccgggcttcagcggcgattgcagctgc

SEQ ID NO: 160 - DNA, 17 bp, synthetic construct
  source 1..17: mol_type = other DNA
gcagctgcgttcgccgc

SEQ ID NO: 161 - DNA, 1970 bp, synthetic construct
  source 1..1970: mol_type = other DNA
actagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagctccatatgccgcggagaggaccaaagaaatagcagagacaaaaaaaaaataaataaaaattaaaaaaaataaaaaaactaattcccggagcccggcgctatccacccatccaccatccatccccctgccagccagccacttatccatccaaccatcttcccatccacacccatccatctcctcatctatgcatccagcgccgggcttcagcggcgattgcagctgcccagttcttgcaatttcctataacacttacgtcaaaacatttccgcagctgcgttcgccgccctcagcccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 162 - DNA, 2207 bp, synthetic construct
  source 1..2207: mol_type = other DNA
cgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcactagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagctccatatgccgcggagaggaccaaagaaatagcagagacaaaaaaaaaataaataaaaattaaaaaaaataaaaaaactaattcccggagcccggcgctatccacccatccaccatccatccccctgccagccagccacttatccatccaaccatcttcccatccacacccatccatctcctcatctatgcatccagcgccgggcttcagcggcgattgcagctgcccagttcttgcaatttcctataacacttacgtcaaaacatttccgcagctgcgttcgccgccctcagcccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 163 - RNA, 1486 nt, synthetic construct
  source 1..1486: mol_type = other RNA
gctttcatttttgtctttttttaacctgggcctgggcctgggtactaacacgatcgtttttttccctttttttccagggaagcggagctactaacttcagcctgctgaagcaggctggagacgtggaggagaaccctgggcctgcttcgtacccctgccatcaacacgcgtctgcgttcgaccaggcggcgcgatcacggggacacagcaaccgacggacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgc

SEQ ID NO: 164 - DNA, 1986 bp, synthetic construct
  source 1..1986: mol_type = other DNA
actagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagctccatatgccgcggagaggaccaaagaaatagcagagacaaaaaaaaaataaataaaaattaaaaaaaataaaaaaactaattcccggagcccggcgctatccacccatccaccatccatccccctgccagccagccacttatccatccaaccatcttcccatccacacccatccatctcctcatctatgcatccagcgccgggcttcagcggcgattgcagctgcccagttcttgcaatttcctataacacttacgtcaaaacatttccgcagctgcgttcgccgccctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 165 - DNA, 2223 bp, synthetic construct
  source 1..2223: mol_type = other DNA
cgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcactagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagctccatatgccgcggagaggaccaaagaaatagcagagacaaaaaaaaaataaataaaaattaaaaaaaataaaaaaactaattcccggagcccggcgctatccacccatccaccatccatccccctgccagccagccacttatccatccaaccatcttcccatccacacccatccatctcctcatctatgcatccagcgccgggcttcagcggcgattgcagctgcccagttcttgcaatttcctataacacttacgtcaaaacatttccgcagctgcgttcgccgccctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 166 - RNA, 1346 nt, synthetic construct
  source 1..1346: mol_type = other RNA
tcagatcgctagctccatatgccgcggagaggaccaaagaaatagcagagacaaaaaaaaaataaataaaaattaaaaaaaataaaaaaactaattcccggagcccggcgctatccacccatccaccatccatccccctgccagccagccacttatccatccaaccatcttcccatccacacccatccatctcctcatctatgcatccagcgccgggcttcagcggcgattgcagctgcccagttcttgcaatttcctataacacttacgtcaaaacatttccgcagctgcgttcgccgccctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgc

SEQ ID NO: 167 - DNA, 303 bp, synthetic construct
  source 1..303: mol_type = other DNA
gctagctcgctcggcaaagtcactgttttcctctcctttccttctaatccctttactcttcctattcctctcttatttttttttttttttttttttttatttttttaatttttttttatggtcttttgtctttttttttgttgtagctatttcttggtttttgctgagcgaatgaaagcgcagagggatcctgtctctgtctctccctgtctccactcaccacccctgcccctactacctgattctctcttggaattattgtctctctgtgtcaaaattaaaaataaaaaaatctacctcagc

SEQ ID NO: 168 - DNA, 110 bp, synthetic construct
  source 1..110: mol_type = other DNA
gatcctgtctctgtctctccctgtctccactcaccacccctgcccctactacctgattctctcttggaattattgtctctctgtgtcaaaattaaaaataaaaaaatcta

SEQ ID NO: 169 - DNA, 141 bp, synthetic construct
  source 1..141: mol_type = other DNA
aagtcactgttttcctctcctttccttctaatccctttactcttcctattcctctcttatttttttttttttttttttttttatttttttaatttttttttatggtcttttgtctttttttttgttgtagctatttcttgg

SEQ ID NO: 170 - DNA, 10 bp, synthetic construct
  source 1..10: mol_type = other DNA
tcgctcggca

SEQ ID NO: 171 - DNA, 29 bp, synthetic construct
  source 1..29: mol_type = other DNA
tttttgctgagcgaatgaaagcgcagagg

SEQ ID NO: 172 - DNA, 1973 bp, synthetic construct
  source 1..1973: mol_type = other DNA
actagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagctcgctcggcaaagtcactgttttcctctcctttccttctaatccctttactcttcctattcctctcttatttttttttttttttttttttttatttttttaatttttttttatggtcttttgtctttttttttgttgtagctatttcttggtttttgctgagcgaatgaaagcgcagagggatcctgtctctgtctctccctgtctccactcaccacccctgcccctactacctgattctctcttggaattattgtctctctgtgtcaaaattaaaaataaaaaaatctacctcagcccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 173 - DNA, 2210 bp, synthetic construct
  source 1..2210: mol_type = other DNA
cgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcactagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagctcgctcggcaaagtcactgttttcctctcctttccttctaatccctttactcttcctattcctctcttatttttttttttttttttttttttatttttttaatttttttttatggtcttttgtctttttttttgttgtagctatttcttggtttttgctgagcgaatgaaagcgcagagggatcctgtctctgtctctccctgtctccactcaccacccctgcccctactacctgattctctcttggaattattgtctctctgtgtcaaaattaaaaataaaaaaatctacctcagcccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 174 - RNA, 1796 nt, synthetic construct
  source 1..1796: mol_type = other RNA
tcagatcgctagctcgctcggcaaagtcactgttttcctctcctttccttctaatccctttactcttcctattcctctcttatttttttttttttttttttttttatttttttaatttttttttatggtcttttgtctttttttttgttgtagctatttcttggtttttgctgagcgaatgaaagcgcagagggatcctgtctctgtctctccctgtctccactcaccacccctgcccctactacctgattctctcttggaattattgtctctctgtgtcaaaattaaaaataaaaaaatctacctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctgcttcgtacccctgccatcaacacgcgtctgcgttcgaccaggcggcgcgatcacggggacacagcaaccgacggacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgc

SEQ ID NO: 175 - DNA, 1989 bp, synthetic construct
  source 1..1989: mol_type = other DNA
actagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagctcgctcggcaaagtcactgttttcctctcctttccttctaatccctttactcttcctattcctctcttatttttttttttttttttttttttatttttttaatttttttttatggtcttttgtctttttttttgttgtagctatttcttggtttttgctgagcgaatgaaagcgcagagggatcctgtctctgtctctccctgtctccactcaccacccctgcccctactacctgattctctcttggaattattgtctctctgtgtcaaaattaaaaataaaaaaatctacctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 176 - DNA, 2226 bp, synthetic construct
  source 1..2226: mol_type = other DNA
cgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcactagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagctcgctcggcaaagtcactgttttcctctcctttccttctaatccctttactcttcctattcctctcttatttttttttttttttttttttttatttttttaatttttttttatggtcttttgtctttttttttgttgtagctatttcttggtttttgctgagcgaatgaaagcgcagagggatcctgtctctgtctctccctgtctccactcaccacccctgcccctactacctgattctctcttggaattattgtctctctgtgtcaaaattaaaaataaaaaaatctacctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 177 - RNA, 1349 nt, synthetic construct
  source 1..1349: mol_type = other RNA
tcagatcgctagctcgctcggcaaagtcactgttttcctctcctttccttctaatccctttactcttcctattcctctcttatttttttttttttttttttttttatttttttaatttttttttatggtcttttgtctttttttttgttgtagctatttcttggtttttgctgagcgaatgaaagcgcagagggatcctgtctctgtctctccctgtctccactcaccacccctgcccctactacctgattctctcttggaattattgtctctctgtgtcaaaattaaaaataaaaaaatctacctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgc

SEQ ID NO: 178 - DNA, 196 bp, synthetic construct
  source 1..196: mol_type = other DNA
gctagctttttccgggccggggggctctctctctctctttcttccttccttccattttttttttttttttccccccggcccggtcagcggcgattgcagctgcgactcatacttctcttctactacctttctatcataaaatctctccacatttcaaacccccgcagctgcgttcgccgcgttcgcctccctcagc

SEQ ID NO: 179 - DNA, 90 bp, synthetic construct
  source 1..90: mol_type = other DNA
ctctctctctctctttcttccttccttccatttttttttttttttttttgtctttagtcttttgttgttgttgttgatgctatttcttgg

SEQ ID NO: 180 - DNA, 98 bp, synthetic construct
  source 1..98: mol_type = other DNA
atcatgcatgcatttttaataaggactcatacttctcttctactacctttctatcataaaatctctccacatttcaaaccccctaaaatctcaagttt

SEQ ID NO: 181 - DNA, 18 bp, synthetic construct
  source 1..18: mol_type = other DNA
tttttccgggccgggggg

SEQ ID NO: 182 - DNA, 33 bp, synthetic construct
  source 1..33: mol_type = other DNA
ccccccggcccggtcagcggcgattgcagctgc

SEQ ID NO: 183 - DNA, 26 bp, synthetic construct
  source 1..26: mol_type = other DNA
gcagctgcgttcgccgcgttcgcctc

SEQ ID NO: 184 - DNA, 1859 bp, synthetic construct
  source 1..1859: mol_type = other DNA
actagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccggctagctttttccgggccggggggctctctctctctctttcttccttccttccattttttttttttttttccccccggcccggtcagcggcgattgcagctgcgactcatacttctcttctactacctttctatcataaaatctctccacatttcaaacccccgcagctgcgttcgccgcgttcgcctccctcagcccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 185 - DNA, 2096 bp, synthetic construct
  source 1..2096: mol_type = other DNA
cgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcactagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccggctagctttttccgggccggggggctctctctctctctttcttccttccttccattttttttttttttttccccccggcccggtcagcggcgattgcagctgcgactcatacttctcttctactacctttctatcataaaatctctccacatttcaaacccccgcagctgcgttcgccgcgttcgcctccctcagcccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 186 - RNA, 1689 nt, synthetic construct
  source 1..1689: mol_type = other RNA
tcagatcgctagctttttccgggccggggggctctctctctctctttcttccttccttccattttttttttttttttccccccggcccggtcagcggcgattgcagctgcgactcatacttctcttctactacctttctatcataaaatctctccacatttcaaacccccgcagctgcgttcgccgcgttcgcctccctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctgcttcgtacccctgccatcaacacgcgtctgcgttcgaccaggcggcgcgatcacggggacacagcaaccgacggacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgc

SEQ ID NO: 187 - DNA, 1875 bp, synthetic construct
  source 1..1875: mol_type = other DNA
actagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccggctagctttttccgggccggggggctctctctctctctttcttccttccttccattttttttttttttttccccccggcccggtcagcggcgattgcagctgcgactcatacttctcttctactacctttctatcataaaatctctccacatttcaaacccccgcagctgcgttcgccgcgttcgcctccctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 188 - DNA, 2112 bp, synthetic construct
  source 1..2112: mol_type = other DNA
cgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcactagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccggctagctttttccgggccggggggctctctctctctctttcttccttccttccattttttttttttttttccccccggcccggtcagcggcgattgcagctgcgactcatacttctcttctactacctttctatcataaaatctctccacatttcaaacccccgcagctgcgttcgccgcgttcgcctccctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 189 - RNA, 1242 nt, synthetic construct
  source 1..1242: mol_type = other RNA
tcagatcgctagctttttccgggccggggggctctctctctctctttcttccttccttccattttttttttttttttccccccggcccggtcagcggcgattgcagctgcgactcatacttctcttctactacctttctatcataaaatctctccacatttcaaacccccgcagctgcgttcgccgcgttcgcctccctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgc

SEQ ID NO: 190 - DNA, 126 bp, synthetic construct
  source 1..126: mol_type = other DNA
gctagagatcggccggggccctctcataccctcatacccacacacttcagggataaaacacttccttttcttttcatttcttttcttttttttttttttttttttagagccggccgatccctcagc

SEQ ID NO: 191 - DNA, 93 bp, synthetic construct
  source 1..93: mol_type = other DNA
ggccctctcataccctcatacccacacacttcagggataaaacacttccttttcttttcatttcttttcttttttttttttttttttttagag

SEQ ID NO: 192 - DNA, 10 bp, synthetic construct
  source 1..10: mol_type = other DNA
gatcggccgg

SEQ ID NO: 193 - DNA, 10 bp, synthetic construct
  source 1..10: mol_type = other DNA
ccggccgatc

SEQ ID NO: 194 - DNA, 1796 bp, synthetic construct
  source 1..1796: mol_type = other DNA
actagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagagatcggccggggccctctcataccctcatacccacacacttcagggataaaacacttccttttcttttcatttcttttcttttttttttttttttttttagagccggccgatccctcagcccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 195 - DNA, 2033 bp, synthetic construct
  source 1..2033: mol_type = other DNA
cgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcactagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagagatcggccggggccctctcataccctcatacccacacacttcagggataaaacacttccttttcttttcatttcttttcttttttttttttttttttttagagccggccgatccctcagcccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 196 - RNA, 1619 nt, synthetic construct
  source 1..1619: mol_type = other RNA
tcagatcgctagagatcggccggggccctctcataccctcatacccacacacttcagggataaaacacttccttttcttttcatttcttttcttttttttttttttttttttagagccggccgatccctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctgcttcgtacccctgccatcaacacgcgtctgcgttcgaccaggcggcgcgatcacggggacacagcaaccgacggacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgc

SEQ ID NO: 197 - DNA, 147 bp, synthetic construct
  source 1..147: mol_type = other DNA
gctagactggaaaaaaagggaaaaaaagatcggccggggccctctcataccctcatacccacacacttcagggataaaacacttccttttcttttcatttcttttcttttttttttttttttttttagagccggccgatccctcagc

SEQ ID NO: 198 - DNA, 1817 bp, synthetic construct
  source 1..1817: mol_type = other DNA
actagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagactggaaaaaaagggaaaaaaagatcggccggggccctctcataccctcatacccacacacttcagggataaaacacttccttttcttttcatttcttttcttttttttttttttttttttagagccggccgatccctcagcccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 199 - DNA, 2054 bp, synthetic construct
  source 1..2054: mol_type = other DNA
cgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcactagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagactggaaaaaaagggaaaaaaagatcggccggggccctctcataccctcatacccacacacttcagggataaaacacttccttttcttttcatttcttttcttttttttttttttttttttagagccggccgatccctcagcccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 200 - RNA, 1640 nt, synthetic construct
  source 1..1640: mol_type = other RNA
tcagatcgctagactggaaaaaaagggaaaaaaagatcggccggggccctctcataccctcatacccacacacttcagggataaaacacttccttttcttttcatttcttttcttttttttttttttttttttagagccggccgatccctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctgcttcgtacccctgccatcaacacgcgtctgcgttcgaccaggcggcgcgatcacggggacacagcaaccgacggacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgc

SEQ ID NO: 201 - DNA, 1812 bp, synthetic construct
  source 1..1812: mol_type = other DNA
actagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagagatcggccggggccctctcataccctcatacccacacacttcagggataaaacacttccttttcttttcatttcttttcttttttttttttttttttttagagccggccgatccctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 202 - DNA, 2049 bp, synthetic construct
  source 1..2049: mol_type = other DNA
cgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcactagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagagatcggccggggccctctcataccctcatacccacacacttcagggataaaacacttccttttcttttcatttcttttcttttttttttttttttttttagagccggccgatccctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 203 - RNA, 1172 nt, synthetic construct
  source 1..1172: mol_type = other RNA
tcagatcgctagagatcggccggggccctctcataccctcatacccacacacttcagggataaaacacttccttttcttttcatttcttttcttttttttttttttttttttagagccggccgatccctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgc

SEQ ID NO: 204 - DNA, 1833 bp, synthetic construct
  source 1..1833: mol_type = other DNA
actagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagactggaaaaaaagggaaaaaaagatcggccggggccctctcataccctcatacccacacacttcagggataaaacacttccttttcttttcatttcttttcttttttttttttttttttttagagccggccgatccctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 205 - DNA, 2070 bp, synthetic construct
  source 1..2070: mol_type = other DNA
cgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcactagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagactggaaaaaaagggaaaaaaagatcggccggggccctctcataccctcatacccacacacttcagggataaaacacttccttttcttttcatttcttttcttttttttttttttttttttagagccggccgatccctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 206 - RNA, 1193 nt, synthetic construct
  source 1..1193: mol_type = other RNA
tcagatcgctagactggaaaaaaagggaaaaaaagatcggccggggccctctcataccctcatacccacacacttcagggataaaacacttccttttcttttcatttcttttcttttttttttttttttttttagagccggccgatccctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgc

SEQ ID NO: 207 - DNA, 165 bp, synthetic construct
  source 1..165: mol_type = other DNA
gctagagactgaaaacaaacaagcagacaaacaaaaaaacatagaagaacagaaacagaaaaaaaaaacatacaaacaaaaaacagaataagaacagaaacaaaaaaacacacacaaaagaaccagaaaaagaaaatgccaacgtgacaccatcaaggcctcagc

SEQ ID NO: 208 - DNA, 152 bp, synthetic construct
  source 1..152: mol_type = other DNA
gactgaaaacaaacaagcagacaaacaaaaaaacatagaagaacagaaacagaaaaaaaaaacatacaaacaaaaaacagaataagaacagaaacaaaaaaacacacacaaaagaaccagaaaaagaaaatgccaacgtgacaccatcaagg

SEQ ID NO: 209 - DNA, 1835 bp, synthetic construct
  source 1..1835: mol_type = other DNA
actagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagagactgaaaacaaacaagcagacaaacaaaaaaacatagaagaacagaaacagaaaaaaaaaacatacaaacaaaaaacagaataagaacagaaacaaaaaaacacacacaaaagaaccagaaaaagaaaatgccaacgtgacaccatcaaggcctcagcccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 210 - DNA, 2072 bp, synthetic construct
  source 1..2072: mol_type = other DNA
cgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcactagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagagactgaaaacaaacaagcagacaaacaaaaaaacatagaagaacagaaacagaaaaaaaaaacatacaaacaaaaaacagaataagaacagaaacaaaaaaacacacacaaaagaaccagaaaaagaaaatgccaacgtgacaccatcaaggcctcagcccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 211 - RNA, 1658 nt, synthetic construct
  source 1..1658: mol_type = other RNA
tcagatcgctagagactgaaaacaaacaagcagacaaacaaaaaaacatagaagaacagaaacagaaaaaaaaaacatacaaacaaaaaacagaataagaacagaaacaaaaaaacacacacaaaagaaccagaaaaagaaaatgccaacgtgacaccatcaaggcctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctgcttcgtacccctgccatcaacacgcgtctgcgttcgaccaggcggcgcgatcacggggacacagcaaccgacggacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgc

SEQ ID NO: 212 - DNA, 192 bp, synthetic construct
  source 1..192: mol_type = other DNA
gctagactggaaaaaaagggaaaaaaatctagagactgaaaacaaacaagcagacaaacaaaaaaacatagaagaacagaaacagaaaaaaaaaacatacaaacaaaaaacagaataagaacagaaacaaaaaaacacacacaaaagaaccagaaaaagaaaatgccaacgtgacaccatcaaggcctcagc

SEQ ID NO: 213 - DNA, 1862 bp, synthetic construct
  source 1..1862: mol_type = other DNA
actagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagactggaaaaaaagggaaaaaaatctagagactgaaaacaaacaagcagacaaacaaaaaaacatagaagaacagaaacagaaaaaaaaaacatacaaacaaaaaacagaataagaacagaaacaaaaaaacacacacaaaagaaccagaaaaagaaaatgccaacgtgacaccatcaaggcctcagcccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 214 - DNA, 2099 bp, synthetic construct
  source 1..2099: mol_type = other DNA
cgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcactagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagactggaaaaaaagggaaaaaaatctagagactgaaaacaaacaagcagacaaacaaaaaaacatagaagaacagaaacagaaaaaaaaaacatacaaacaaaaaacagaataagaacagaaacaaaaaaacacacacaaaagaaccagaaaaagaaaatgccaacgtgacaccatcaaggcctcagcccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 215 - RNA, 1685 nt, synthetic construct
  source 1..1685: mol_type = other RNA
tcagatcgctagactggaaaaaaagggaaaaaaatctagagactgaaaacaaacaagcagacaaacaaaaaaacatagaagaacagaaacagaaaaaaaaaacatacaaacaaaaaacagaataagaacagaaacaaaaaaacacacacaaaagaaccagaaaaagaaaatgccaacgtgacaccatcaaggcctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctgcttcgtacccctgccatcaacacgcgtctgcgttcgaccaggcggcgcgatcacggggacacagcaaccgacggacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgc

SEQ ID NO: 216 - DNA, 1850 bp, synthetic construct
  source 1..1850: mol_type = other DNA
actagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagagactgaaaacaaacaagcagacaaacaaaaaaacatagaagaacagaaacagaaaaaaaaaacatacaaacaaaaaacagaataagaacagaaacaaaaaaacacacacaaaagaaccagaaaaagaaaatgccaacgtgacaccatcaaggcctcaggctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 217 - DNA, 2087 bp, synthetic construct
  source 1..2087: mol_type = other DNA
cgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcactagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagagactgaaaacaaacaagcagacaaacaaaaaaacatagaagaacagaaacagaaaaaaaaaacatacaaacaaaaaacagaataagaacagaaacaaaaaaacacacacaaaagaaccagaaaaagaaaatgccaacgtgacaccatcaaggcctcaggctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 218 - RNA, 1210 nt, synthetic construct
  source 1..1210: mol_type = other RNA
tcagatcgctagagactgaaaacaaacaagcagacaaacaaaaaaacatagaagaacagaaacagaaaaaaaaaacatacaaacaaaaaacagaataagaacagaaacaaaaaaacacacacaaaagaaccagaaaaagaaaatgccaacgtgacaccatcaaggcctcaggctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgc

SEQ ID NO: 219 - DNA, 1877 bp, synthetic construct
  source 1..1877: mol_type = other DNA
actagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagactggaaaaaaagggaaaaaaatctagagactgaaaacaaacaagcagacaaacaaaaaaacatagaagaacagaaacagaaaaaaaaaacatacaaacaaaaaacagaataagaacagaaacaaaaaaacacacacaaaagaaccagaaaaagaaaatgccaacgtgacaccatcaaggcctcaggctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 220 - DNA, 2114 bp, synthetic construct
  source 1..2114: mol_type = other DNA
cgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcactagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagactggaaaaaaagggaaaaaaatctagagactgaaaacaaacaagcagacaaacaaaaaaacatagaagaacagaaacagaaaaaaaaaacatacaaacaaaaaacagaataagaacagaaacaaaaaaacacacacaaaagaaccagaaaaagaaaatgccaacgtgacaccatcaaggcctcaggctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 221 - RNA, 1237 nt, synthetic construct
  source 1..1237: mol_type = other RNA
tcagatcgctagactggaaaaaaagggaaaaaaatctagagactgaaaacaaacaagcagacaaacaaaaaaacatagaagaacagaaacagaaaaaaaaaacatacaaacaaaaaacagaataagaacagaaacaaaaaaacacacacaaaagaaccagaaaaagaaaatgccaacgtgacaccatcaaggcctcaggctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgc

SEQ ID NO: 222 - DNA, 278 bp, synthetic construct
  source 1..278: mol_type = other DNA
gctagagactgaaaacaaacaagcagacaaacaaaaaaacatagaagaacagaaacagaaaaaaaaaacatacaaacaaaaaacagaataagaacagaaacaaaaaaacacacacaaaagaaccagaaaaagaaaatgccaacgtgacaccatcaagggatcggccggggccctctcataccctcatacccacacacttcagggataaaacacttccttttcttttcatttcttttcttttttttttttttttttttagagccggccgatccctcagc

SEQ ID NO: 223 - DNA, 152 bp, synthetic construct; identical to SEQ ID NO: 208
  source 1..152: mol_type = other DNA

SEQ ID NO: 224 - DNA, 93 bp, synthetic construct; identical to SEQ ID NO: 191
  source 1..93: mol_type = other DNA

SEQ ID NO: 225 - DNA, 10 bp, synthetic construct
  source 1..10: mol_type = other DNA
gatcggccgg

SEQ ID NO: 226 - DNA, 10 bp, synthetic construct
  source 1..10: mol_type = other DNA
ccggccgatc

SEQ ID NO: 227 - DNA, 1948 bp, synthetic construct
  source 1..1948: mol_type = other DNA
actagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagagactgaaaacaaacaagcagacaaacaaaaaaacatagaagaacagaaacagaaaaaaaaaacatacaaacaaaaaacagaataagaacagaaacaaaaaaacacacacaaaagaaccagaaaaagaaaatgccaacgtgacaccatcaagggatcggccggggccctctcataccctcatacccacacacttcagggataaaacacttccttttcttttcatttcttttcttttttttttttttttttttagagccggccgatccctcagcccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 228 - DNA, 2185 bp, synthetic construct
  source 1..2185: mol_type = other DNA
cgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcactagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagagactgaaaacaaacaagcagacaaacaaaaaaacatagaagaacagaaacagaaaaaaaaaacatacaaacaaaaaacagaataagaacagaaacaaaaaaacacacacaaaagaaccagaaaaagaaaatgccaacgtgacaccatcaagggatcggccggggccctctcataccctcatacccacacacttcagggataaaacacttccttttcttttcatttcttttcttttttttttttttttttttagagccggccgatccctcagcccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 229 - RNA, 1771 nt, synthetic construct
  source 1..1771: mol_type = other RNA
tcagatcgctagagactgaaaacaaacaagcagacaaacaaaaaaacatagaagaacagaaacagaaaaaaaaaacatacaaacaaaaaacagaataagaacagaaacaaaaaaacacacacaaaagaaccagaaaaagaaaatgccaacgtgacaccatcaagggatcggccggggccctctcataccctcatacccacacacttcagggataaaacacttccttttcttttcatttcttttcttttttttttttttttttttagagccggccgatccctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctgcttcgtacccctgccatcaacacgcgtctgcgttcgaccaggcggcgcgatcacggggacacagcaaccgacggacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgc

SEQ ID NO: 230 - DNA, 305 bp, synthetic construct
  source 1..305: mol_type = other DNA
gctagactggaaaaaaagggaaaaaaatctagagactgaaaacaaacaagcagacaaacaaaaaaacatagaagaacagaaacagaaaaaaaaaacatacaaacaaaaaacagaataagaacagaaacaaaaaaacacacacaaaagaaccagaaaaagaaaatgccaacgtgacaccatcaagggatcggccggggccctctcataccctcatacccacacacttcagggataaaacacttccttttcttttcatttcttttcttttttttttttttttttttagagccggccgatccctcagc

SEQ ID NO: 231 - DNA, 1975 bp, synthetic construct
  source 1..1975: mol_type = other DNA
actagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagactggaaaaaaagggaaaaaaatctagagactgaaaacaaacaagcagacaaacaaaaaaacatagaagaacagaaacagaaaaaaaaaacatacaaacaaaaaacagaataagaacagaaacaaaaaaacacacacaaaagaaccagaaaaagaaaatgccaacgtgacaccatcaagggatcggccggggccctctcataccctcatacccacacacttcagggataaaacacttccttttcttttcatttcttttcttttttttttttttttttttagagccggccgatccctcagcccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 232 - DNA, 2212 bp, synthetic construct
  source 1..2212: mol_type = other DNA
cgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcactagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagactggaaaaaaagggaaaaaaatctagagactgaaaacaaacaagcagacaaacaaaaaaacatagaagaacagaaacagaaaaaaaaaacatacaaacaaaaaacagaataagaacagaaacaaaaaaacacacacaaaagaaccagaaaaagaaaatgccaacgtgacaccatcaagggatcggccggggccctctcataccctcatacccacacacttcagggataaaacacttccttttcttttcatttcttttcttttttttttttttttttttagagccggccgatccctcagcccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 233 - RNA, 1798 nt, synthetic construct
  source 1..1798: mol_type = other RNA
tcagatcgctagactggaaaaaaagggaaaaaaatctagagactgaaaacaaacaagcagacaaacaaaaaaacatagaagaacagaaacagaaaaaaaaaacatacaaacaaaaaacagaataagaacagaaacaaaaaaacacacacaaaagaaccagaaaaagaaaatgccaacgtgacaccatcaagggatcggccggggccctctcataccctcatacccacacacttcagggataaaacacttccttttcttttcatttcttttcttttttttttttttttttttagagccggccgatccctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctgcttcgtacccctgccatcaacacgcgtctgcgttcgaccaggcggcgcgatcacggggacacagcaaccgacggacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccaggtaagtatcaaggttacaagacaggtttaaggagaccaatagaaactgggcttgtcgagacagagacgactcttgcgtttctgataggcacctattggtcttactgacatccactttgcctttctctccacagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccaccccaggctccataccgacgatctgcgacctggcgcgcacgtttgcgcgggagatgggggaggcgaactgaaacttgtttattgcagcttataatggttacaaataaagcaatagcatcacaaatttcacaaataaagcatttttttcactgc

SEQ ID NO: 234 - DNA, 1964 bp, synthetic construct
  source 1..1964: mol_type = other DNA
actagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagagactgaaaacaaacaagcagacaaacaaaaaaacatagaagaacagaaacagaaaaaaaaaacatacaaacaaaaaacagaataagaacagaaacaaaaaaacacacacaaaagaaccagaaaaagaaaatgccaacgtgacaccatcaagggatcggccggggccctctcataccctcatacccacacacttcagggataaaacacttccttttcttttcatttcttttcttttttttttttttttttttagagccggccgatccctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 235 - DNA, 2201 bp, synthetic construct
  source 1..2201: mol_type = other DNA
cgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcactagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagagactgaaaacaaacaagcagacaaacaaaaaaacatagaagaacagaaacagaaaaaaaaaacatacaaacaaaaaacagaataagaacagaaacaaaaaaacacacacaaaagaaccagaaaaagaaaatgccaacgtgacaccatcaagggatcggccggggccctctcataccctcatacccacacacttcagggataaaacacttccttttcttttcatttcttttcttttttttttttttttttttagagccggccgatccctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 236 - RNA, 1324 nt, synthetic construct
  source 1..1324: mol_type = other RNA
tcagatcgctagagactgaaaacaaacaagcagacaaacaaaaaaacatagaagaacagaaacagaaaaaaaaaacatacaaacaaaaaacagaataagaacagaaacaaaaaaacacacacaaaagaaccagaaaaagaaaatgccaacgtgacaccatcaagggatcggccggggccctctcataccctcatacccacacacttcagggataaaacacttccttttcttttcatttcttttcttttttttttttttttttttagagccggccgatccctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgc

SEQ ID NO: 237 - DNA, 1991 bp, synthetic construct
  source 1..1991: mol_type = other DNA
actagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagactggaaaaaaagggaaaaaaatctagagactgaaaacaaacaagcagacaaacaaaaaaacatagaagaacagaaacagaaaaaaaaaacatacaaacaaaaaacagaataagaacagaaacaaaaaaacacacacaaaagaaccagaaaaagaaaatgccaacgtgacaccatcaagggatcggccggggccctctcataccctcatacccacacacttcagggataaaacacttccttttcttttcatttcttttcttttttttttttttttttttagagccggccgatccctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 238 - DNA, 2228 bp, synthetic construct
  source 1..2228: mol_type = other DNA
cgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcactagttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgctagactggaaaaaaagggaaaaaaatctagagactgaaaacaaacaagcagacaaacaaaaaaacatagaagaacagaaacagaaaaaaaaaacatacaaacaaaaaacagaataagaacagaaacaaaaaaacacacacaaaagaaccagaaaaagaaaatgccaacgtgacaccatcaagggatcggccggggccctctcataccctcatacccacacacttcagggataaaacacttccttttcttttcatttcttttcttttttttttttttttttttagagccggccgatccctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgcattctagttgtggtttgtccaaactcatcaatgtatctgaagacaatagcag

SEQ ID NO: 239 - RNA, 1351 nt, synthetic construct
  source 1..1351: mol_type = other RNA
tcagatcgctagactggaaaaaaagggaaaaaaatctagagactgaaaacaaacaagcagacaaacaaaaaaacatagaagaacagaaacagaaaaaaaaaacatacaaacaaaaaacagaataagaacagaaacaaaaaaacacacacaaaagaaccagaaaaagaaaatgccaacgtgacaccatcaagggatcggccggggccctctcataccctcatacccacacacttcagggataaaacacttccttttcttttcatttcttttcttttttttttttttttttttagagccggccgatccctcagcgctttcatttttgtctttttttaacctgggcctgggcctgggtactaacacgatcgtttttttccctttttttccagggaagcggagctactaacttcagcctgctgaagcaggctggagacgtggaggagaaccctgggcctcccgccatgaaggtaagtatcaaggttacaagacaggtttaaggagaccaatagaaactgggcttgtcgagacagagacgactcttgcgtttctgataggcacctattggtcttactgacatccactttgcctttctctccacagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgaaacttgtttattgcagcttataatggttacaaataaagcaatagcatcacaaatttcacaaataaagcatttttttcactgc

SEQ ID NO: 240 - DNA, 508 bp, synthetic construct
  source 1..508: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 241 - DNA, 133 bp, synthetic construct
  source 1..133: mol_type = other DNA
gtaagtatcaaggttacaagacaggtttaaggagaccaatagaaactgggcttgtcgagacagagacgactcttgcgtttctgataggcacctattggtcttactgacatccactttgcctttctctccacag

SEQ ID NO: 242 - DNA, 21 bp, synthetic construct
  source 1..21: mol_type = other DNA
gttctcatgcacacttatagc

SEQ ID NO: 243 - DNA, 35 bp, synthetic construct
  source 1..35: mol_type = other DNA
  misc_feature 15: note = dSpacer between residues 15 and 16
atccagttttctggagactcttcgcgatgtacggg

SEQ ID NO: 244 - DNA, 34 bp, synthetic construct
  source 1..34: mol_type = other DNA
  misc_feature 15: note = dSpacer between residues 15 and 16
aaggtcttttgacctgaagccatagagcccaccg

SEQ ID NO: 245 - DNA, 93 bp, synthetic construct; identical to SEQ ID NO: 6
  source 1..93: mol_type = other DNA
  misc_feature 72: note = AP-1 binding site between residues 72 and 73 having has a nucleotide sequence of ATGAGTCAT

SEQ ID NO: 246 - DNA, 123 bp, synthetic construct
  source 1..123: mol_type = other DNA
  misc_feature 102: note = AP-1 binding site between residues 102 and 103 having has a nucleotide sequence of ATGAGTCAT
atccagttttattttattttattttagttctcatgcacacttatagcgggaaacccgttctcatgcacacttatagcggtttggtttggtttggtttctggagcgatgtacgggccagatata

SEQ ID NO: 247 - DNA, 18 bp, synthetic construct
  source 1..18: mol_type = other DNA
atcgagtgccgcatcacc

SEQ ID NO: 248 - DNA, 22 bp, synthetic construct
  source 1..22: mol_type = other DNA
actcatcgagctcgagatctgg

SEQ ID NO: 249 - DNA, 18 bp, synthetic construct
  source 1..18: mol_type = other DNA
ctggcacccagcacaatg

SEQ ID NO: 250 - DNA, 20 bp, synthetic construct
  source 1..20: mol_type = other DNA
gccgatccacacggagtact